(19) United States
(12) Patent Application Publication
(10) Pub. No.: US 2008/0255183 A1
(43) Pub. Date: Oct. 16, 2008

(54) N-[HETEROARYLCARBONYL]-3-THIENYL-L-ALANINE DERIVATIVES AS a5beta1 ANTAGONISTS

(75) Inventors: Jean-Claude ARNOULD, Reims Cedex 2 (FR); Benedicte DELOUVRIE, Reims Cedex 2 (FR); Craig Steven HARRIS, Reims Cedex 2 (FR)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 12/101,335

(22) Filed: Apr. 11, 2008

(30) Foreign Application Priority Data
Apr. 11, 2007 (EP) 07290445.1
Jul. 12, 2007 (EP) 07301228.8
Oct. 17, 2007 (EP) 07301476.3

(51) Int. Cl. C07D 409/14; A61K 31/4375; C07D 487/04; A61P 35/00; A61P 9/00; A61K 31/4439

(52) U.S. Cl. 514/300; 546/271.4; 514/340; 546/122

(57) ABSTRACT

The present invention relates to compounds that inhibit of a5b1 function, processes for their preparation, pharmaceutical compositions containing them as the active ingredient, to their use as medicaments and to their use in the manufacture of medicaments for use in the treatment in warm-blooded animals such as humans of diseases that have a significant angiogenesis or vascular component such as for treatment of solid tumours. The present invention also relates to a5b1 antagonists that also exhibit appropriate selectivity profile(s) against other integrins.

The present invention relates to chemical compounds useful as pharmaceuticals in particular in the treatment of diseases in which α5β1 function is a factor, to process for their preparation, and to compositions containing these as well as their use in therapy.

### RELATED APPLICATIONS

This application claims the benefit under 35 USC § 119(a)-(d) of European Patent Application No. EP 07290445.1, filed on Apr. 11, 2007; European Patent Application No. EP 07301228.8, filed on Jul. 12, 2007 and European Patent Application No. EP 07301476.3, filed on Oct. 17, 2007.

### BACKGROUND OF THE INVENTION

Many normal physiological and disease processes require cells to contact other cells and/or extracellular matrix. Cell-matrix and cell-cell adhesion is mediated through several families of proteins including integrins, selecting, cadherins, and immunoglobulins, and facilitates a variety of normal cellular functions such as proliferation, migration, differentiation or survival. Cell adhesion is also key to a range of pathologies, and so pharmacological disruption of cell adhesion interactions can provide a mechanism for therapeutic intervention. In particular members of the integrin superfamily of adhesion molecules are believed to play a particularly important role in acute and chronic disease states such as cancer, inflammatory diseases, stroke and neurodegenerative disorders^{(1,2)}. Thus, integrins represent a very complex biological area.

The integrin superfamily of cell surface receptors is formed from a number of structurally and functionally related surface glycoproteins, with each receptor existing as a heterodimer of non-covalently linked α and β subunits. To date, at least 18 different α and 8 β subunits have been identified in mammals, which are known to form more than 24 different receptors. Each integrin interacts specifically with defined extracellular ligands, including extracellular matrix proteins such as, fibronectin, fibrinogen, vitronectin, collagen and cell surface molecules such as VCAM, ICAM and PECAM, via linear adhesion motifs.

The integrin α5β1 (hereinafter a5b1) is composed of an a5 (hereinafter a5) and β1 (hereinafter b1) subunits, the a5 subunit forming a specific dimer with the b1 subunit, and is widely expressed in most tissues⁽³⁾. Integrin a5b1 almost exclusively mediates cell adhesion through an interaction with fibronectin, binding via the short arginine-glycine-aspartate (RGD) adhesion motif Endothelial cells can however bind to fibrin via a5b1. There is compelling evidence that the a5b1 interaction with fibronectin plays an important role in physiopathological angiogenesis and vascular integrity^{(4,5)}. Although endothelial cells express a variety of integrins, a5b1 is important for survival of endothelial cells on provisional matrix in vitro, suppressing apoptosis and promoting proliferation. Furthermore, immunohistochemical analysis, and imaging have both shown that a5b1 expression is upregulated in tumour vasculature^{(4,6)}. Consistent with a key functional role for the receptor-ligand pairing, the a5b1 ligand fibronectin is also upregulated in tumour tissue and during wound-healing⁽⁴⁾. Transgenic studies further support an important role for a5b1 in the vasculature. Both a5 and b1 knock-out mice are embryonic lethal and display defects in development of early vascular systems, suggesting a pivotal functional role in early vasculogenesis^{(7,8)}. Moreover, studies using agents such as blocking RGD peptides or neutralising antibodies have shown that disruption of a5b1 interaction with its cognate ligands has anti-angiogenic effects in vivo⁽⁴⁾. As well as inhibiting angiogenesis, a5b1 inhibitors may reduce the proliferation of certain tumour cells that express the receptor.

In addition to a5b1, other integrin family members such as avb3 and aiibb3 can also interact with RGD-containing ligands⁽¹⁾. Other integrins can bind to ligands via non-RGD binding domains. An example of particular importance and relevance is a4b1 which binds via a leucine-aspartate-valine (LDV) motif to ligands that include the connecting segment-1 region of fibronectin, VCAM-1, MAdCAM or to the SVVYGLR motif found within osteopontin.

Since there are a variety of integrins that share the same ligand or binding-domain with a5b1, it will be important to develop therapeutic agents that are selective towards a5b1 activity. However, as other endothelial integrins such as avb3, avb5 and a4b1 are also involved in possible pathological events, agents which target such integrins in addition to a5b1, may have additional therapeutic activity.

### REFERENCES

1. Shimaoka, M et al. (2003) Nat. Rev. Drug. Disc. 2, 703-716
2. Mousa, S A (2002) Curr. Opin. Chem. Biol. 6:534-541
3. Parsons-Wingerter, P et al. (2005) Am. J. Path. 167(1), 193-211
4. Kim, S et al. (2000) Am. J. Path. 156(4), 1345-1362
5. Ramakrishnan, V et al. (2006) J. Exp. Ther. One. 5, 273-286
6. Magnussen, A et al. (2005) Cancer Res. 65(7), 2712-2721
7. Goh, K L et al. (1997) Development 124, 4309-4319
8. Yang, J T et al. (1993) Development 119, 1093-1105

Taken together, the expression and functional data suggest that selective inhibition of a5b1 function provides an attractive therapeutic strategy to combat diseases that have a significant angiogenesis or vascular component such as for treatment of solid tumours or other pathological angiogenic conditions such as age-related macular degeneration.

A number of small-molecule a5b1 antagonists are known, for example WO97/33887 describe spirocyclic compounds, and WO2005/090329 describes substituted pyrrolidines and other cyclic and heterocyclic compounds. There are a number of a5b1 antagonists in development, for example JSM6427 and SJ749. There remains however the need to develop alternative a5b1 antagonists.

There is thus a clear need to develop compounds that are a5b1 antagonists with appropriate pharmacokinetic and pharmacodynamic drug properties (such as but not limited to, high bioavailability), and also that exhibit appropriate selectivity profile(s) against other integrins.

### SUMMARY OF THE INVENTION

According to a first aspect of the present invention there is provided a compound of formula I:

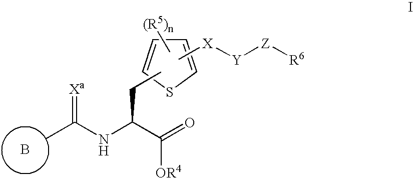

wherein:

X^{a} is selected from oxygen or sulphur;

B is heteroaryl which is substituted ortho to the C(X^{a}) group in formula I by R¹ and optionally bears one or more R³ substituents, wherein R¹ and each R³ are independently selected from halo, trifluoromethyl, cyano, isocyano, nitro, hydroxy, mercapto, amino, formyl, carboxy, carbamoyl, sulfamoyl, halo-(1-3C)alkyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

or from a group of the formula:

Q¹-X¹—

wherein X¹ is a direct bond or is selected from O, S, SO, SO₂, N(R⁷), C(O), CH(OR⁷), C(O)N(R⁷), N(R⁷)C(O), SO₂N(R⁷), N(R⁷)SO₂, OC(R⁷)₂, SC(R⁷)₂ and N(R⁷)C(R⁷)₂, wherein R⁷ is hydrogen or (1-6C)alkyl, and Q¹ is aryl, aryl-(1-6C)alkyl, (3-7C)cycloalkyl, (3-7C)cycloalkyl-(1-6C)alkyl, (3-7C)cycloalkenyl, (3-7C)cycloalkenyl-(1-6C)alkyl, heteroaryl, heteroaryl-(1-6C)alkyl, heterocyclyl or heterocyclyl-(1-6C)alkyl,

and wherein any carbon containing substituent on ring B optionally bears on carbon one or more R⁸ groups,

and wherein if any heteroaryl or heterocyclyl group which is a substituent on ring B contains an —NH— moiety, the nitrogen of said moiety optionally bears a group selected from R⁹,

and wherein any heterocyclyl group which is a substituent on ring B optionally bears 1 or 2 oxo or thioxo substituents;

or two adjacent substituents on ring B optionally form a (1-3C)alkylenedioxy group;

and wherein ring B is linked to the C(X^{a}) group by a ring carbon atom;

R⁴ is selected from hydrogen, (1-6C)alkyl, aryl, aryl-(1-6C)alkyl, heterocyclyl, heteroaryl, heterocyclyl(1-6)alkyl and heteroaryl-(1-6C)alkyl, which optionally bears on carbon one or more R²¹ substituents, which may be the same or different,

and wherein if any heteroaryl or heterocyclyl group within R⁴ contains an —NH— moiety, the nitrogen of said moiety optionally bears a group selected from R²²,

and wherein and wherein any heterocyclyl group within R⁴ optionally bears 1 or 2 oxo or thioxo substituents;

n is 0, 1 or 2;

each R⁵, which may be the same or different, is selected from halo, trifluoromethyl, cyano, isocyano, nitro, hydroxy, mercapto, amino, formyl, carboxy, carbamoyl, sulfamoyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

or from a group of the formula:

Q⁵-X⁷—

wherein X⁷ is a direct bond or is selected from O, S, SO, SO₂, N(R²³), C(O), CH(OR²³), C(O)N(R²³), N(R²³)C(O), SO₂N(R²³), N(R²³)SO₂, OC(R²³)₂, SC(R²³)₂ and N(R²³)C(R²³)₂,

wherein R²³ is hydrogen or (1-6C)alkyl, and Q⁵ is aryl, aryl-(1-6C)alkyl, (3-7C)cycloalkyl, (3-7C)cycloalkyl-(1-6C)alkyl, (3-7C)cycloalkenyl, (3-7C)cycloalkenyl-(1-6C)alkyl, heteroaryl, heteroaryl-(1-6C)alkyl, heterocyclyl or heterocyclyl-(1-6C)alkyl,

and wherein R⁵ optionally bears on carbon one or more R²⁴ groups,

and wherein any if any heteroaryl or heterocyclyl group within R⁵ contains an —NH— moiety, the nitrogen of said moiety optionally bears a group selected from R²⁵,

and wherein any heterocyclyl group within R⁵ optionally bears 1 or 2 oxo or thioxo substituents;

or two R⁵ substituents optionally form a (1-3C)alkylenedioxy group;

with the proviso that when X⁷ is a direct bond then Q⁵ is not aryl or heteroaryl;

X and Z, which may be the same or different, are selected from a direct bond, N(R²⁶), O, S, SO, SO₂, C(O), CH(OR²⁶), C(O)N(R²⁶), N(R²⁶)C(O), SO₂N(R²⁶), N(R²⁶)SO₂, (1-6C)alkylene, CH═CH and C—C, wherein R²⁶ is hydrogen, (1-6C)alkyl or (3-7C)cycloalkyl;

Y is selected from (1-6C)alkylene, (3-7C)cycloalkylene and (3-7C)cycloalkenylene,

and wherein adjacent carbon atoms in any (2-6C)alkylene chain within an X, Y or Z substituent are optionally separated by the insertion into the chain of a group selected from O, S, SO, SO₂, N(R^{27a}), C(O), CH(OR²⁷), C(O)N(R²⁷), N(R²⁷)C(O), SO₂N(R²⁷), N(R²⁷)SO₂, CH═CH and C≡C wherein R²⁷ is hydrogen, (1-6C)alkyl or (3-7C)cycloalkyl, and R^{27a} is hydrogen, (1-6C)alkyl or (3-7C)cycloalkyl, (1-3C)alkoxy(1-3C)alkyl, C(O)R^{27b} S(O)R^{27b} or S(O)₂R^{27b} where R^{27b} is hydrogen, (1-3C)alkyl, (3-7C)cycloalkyl, (1-3C)alkoxy(1-3C)alkyl;

and wherein any X, Y or Z optionally bears on carbon one or more R²⁸ substituents,

R⁶ is heteroaryl, which heteroaryl contains at least one —N═ ring atom,

wherein R⁶ is linked to the group Z by a carbon atom in R⁶,

and wherein R⁶ optionally bears on carbon one or more R³¹ substituents,

and wherein if R⁶ contains an —NH— moiety, the nitrogen of said moiety optionally bears a group selected from R³⁵,

and wherein:

(i) R⁶ is a bicyclic or polycyclic heteroaryl which contains at least one unsubstituted —NH— ring member in addition to the —N═ ring atom, wherein the —NH— and ═N— group in R⁶ are attached to the same bridgehead ring atom at a junction of two fused rings in R⁶; or

(ii) R⁶ is substituted in an ortho position to the —N═ atom in R⁶ by an —NHR^{31a} group; or

(iii) Z is NH and R⁶ is attached to Z by a ring carbon atom in an ortho position to the —N═ atom in R⁶;

and wherein the group Z-R⁶ has a pKa of greater than or equal to about 6;

R⁸, R²¹, R²⁴ and R²⁸ is selected from halo, trifluoromethyl, cyano, nitro, hydroxy, amino, carboxy, carbamoyl, sulfamoyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

or from a group of the formula:

—X²—R¹⁰

wherein X² is a direct bond or is selected from O, C(O) and N(R¹¹), wherein R¹¹ is hydrogen or (1-6C)alkyl, and R¹⁰ is halo-(1-6C)alkyl, hydroxy-(1-6C)alkyl, (1-6C)alkoxy-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl and (1-6C)alkoxycarbonylamino-(1-6C)alkyl,

or from a group of the formula:

—X³-Q²

wherein X³ is a direct bond or is selected from O, S, SO, SO₂, N(R¹²), C(O), CH(OR¹²), C(O)N(R¹²), N(R¹²)C(O), SO₂N(R¹²), N(R¹²)SO₂, OC(R¹²)₂, SC(R¹²)₂ and N(R¹²)C(R¹²)₂,

wherein R¹² is hydrogen or (1-6C)alkyl, and Q² is aryl, aryl-(1-6C)alkyl, (3-7C)cycloalkyl, (3-7C)cycloalkyl-(1-6C)alkyl, (3-7C)cycloalkenyl, (3-7C)cycloalkenyl-(1-6C)alkyl, heteroaryl, heteroaryl-(1-6C)alkyl, heterocyclyl or heterocyclyl-(1-6C)alkyl,

and wherein R⁸, R²¹, R²⁴ and R²⁸ independently of each other optionally bears on carbon one or more R¹³,

and wherein any if any heteroaryl or heterocyclyl group within R⁸, R²¹, R²⁴ and

R²⁸ contains an —NH— moiety, the nitrogen of said moiety optionally bears a group selected from R¹⁴,

and wherein any heterocyclyl group within a substituent on R⁸, R²¹, R²⁴ and R²⁸ independently of each other optionally bears 1 or 2 oxo or thioxo substituents;

R⁹, R²² and R²⁵ are each independently selected from carbamoyl, sulfamoyl, (1-6C)alkyl, (2-8C)alkenyl, (2-8C)alkynyl, (1-6C)alkylsulfonyl, (1-6C)alkoxycarbonyl, N-(1-6C)alkylcarbamoyl, N,N-di-[(1-6C)alkyl]carbamoyl, (2-6C)alkanoyl, N-(1-6C)alkylsulfamoyl and N,N-di-[(1-6C)alkyl]sulfamoyl,

or from a group of the formula:

—X⁴—R¹⁵

wherein X⁴ is a direct bond or is selected from C(O), SO₂, C(O)N(R¹⁶) and SO₂N(R¹⁶),

wherein R¹⁶ is hydrogen or (1-6C)alkyl, and R¹⁵ is halo-(1-6C)alkyl, hydroxy-(1-6C)alkyl, (1-6C)alkoxy-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl and (1-6C)alkoxycarbonylamino-(1-6C)alkyl,

or from a group of the formula:

—X⁵-Q³

wherein X⁵ is a direct bond or is selected from C(O), SO₂, C(O)N(R¹⁷) and SO₂N(R¹⁷), wherein R¹⁷ is hydrogen or (1-6C)alkyl, and Q³ is aryl, aryl-(1-6C)alkyl, (3-7C)cycloalkyl, (3-7C)cycloalkyl-(1-6C)alkyl, (3-7C)cycloalkenyl, (3-7C)cycloalkenyl-(1-6C)alkyl, heteroaryl, heteroaryl-(1-6C)alkyl, heterocyclyl or heterocyclyl-(1-6C)alkyl,

and wherein R⁹, R²² and R²⁵ independently of each other optionally bears on carbon one or more R¹⁸,

and wherein any if any heteroaryl or heterocyclyl group within R⁹, R²² and R²⁵ contains an —NH— moiety, the nitrogen of said moiety optionally bears a group selected from R¹⁹,

and wherein any heterocyclyl group within a substituent on R⁹, R²² and R²⁵ optionally bears 1 or 2 oxo or thioxo substituents;

R¹³ and R¹⁸ are each independently selected from halo, cyano, hydroxy, carboxy, amino, (3-6C)cycloalkyl, (2-6C)alkenyl, (2-6C)alkynyl, (1-6C)alkoxy, (1-6C)alkylamino and di-[(1-6C)alkyl]amino;

R¹⁴ and R¹⁹ are each independently selected from carbamoyl, sulfamoyl, (1-6C)alkyl, (2-8C)alkenyl, (2-8C)alkynyl, (1-6C)alkylsulfonyl, N-(1-6C)alkylcarbamoyl, N,N-di-[(1-6C)alkyl]carbamoyl, (2-6C)alkanoyl, N-(1-6C)alkylsulfamoyl and N,N-di-[(1-6C)alkyl]sulfamoyl,

or from a group of the formula:

—X⁶-Q⁴

wherein X⁶ is a direct bond or is selected from C(O), SO₂, C(O)N(R²⁰) and SO₂N(R²⁰), wherein R²⁰ is hydrogen or (1-6C)alkyl, and Q⁴ is (3-7C)cycloalkyl or (3-7C)cycloalkyl-(1-6C)alkyl;

R³¹ is selected from halo, cyano, hydroxy, nitro, amino, carbamoyl, sulfamoyl, (1-6C)alkyl, (2-6C)alkenyl, (2-6C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (2-6C)alkanoyl, N-(1-6C)alkylcarbamoyl, N,N-di-[(1-6C)alkyl]carbamoyl, (1-6C)alkoxycarbonyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino

or from a group of the formula:

—X⁸—R³²

wherein X⁸ is a direct bond or is selected from O, C(O) and N(R³³), wherein R³³ is hydrogen or (1-6C)alkyl, and R³² is halo-(1-6C)alkyl, hydroxy-(1-6C)alkyl, (1-6C)alkoxy-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl and (1-6C)alkoxycarbonylamino-(1-6C)alkyl,

or from a group of the formula:

—X⁹-Q⁶

wherein X⁹ is a direct bond or is selected from O, S, SO, SO₂, C(O), N(R³⁴), C(O)N(R³⁴), N(R³⁴)C(O), SO₂N(R³⁴), N(R³⁴)SO₂ wherein R³⁴ is hydrogen or (1-6C)alkyl, and Q⁶ is aryl, aryl-(1-6C)alkyl, (3-7C)cycloalkyl, (3-7C)cycloalkyl-(1-6C)alkyl, heteroaryl, heteroaryl-(1-6C)alkyl, heterocyclyl or heterocyclyl-(1-6C)alkyl,

and wherein R³¹ optionally bears on carbon one or more R³⁶,

and wherein any if any heteroaryl or heterocyclyl group within R³¹ contains an —NH— moiety, the nitrogen of said moiety optionally bears a group selected from R³⁷, and wherein any heterocyclyl group within a substituent on R³¹ optionally bears 1 or 2 oxo or thioxo substituents;

R^{31a} is selected from hydrogen, (1-6C)alkyl, (2-8C)alkenyl, (2-8C)alkynyl,

halo-(1-6C)alkyl, hydroxy-(2-6C)alkyl, (1-6C)alkoxy-(2-6C)alkyl, amino-(2-6C)alkyl, (1-6C)alkylamino-(2-6C)alkyl, di-[(1-6C)alkyl]amino-(2-6C)alkyl, (3-7C)cycloalkyl and (3-7C)cycloalkyl-(1-6C)alkyl,

and wherein any (3-7C)cycloalkyl in R^{31a} optionally bears 1 or more (1-6C)alkyl substituents;

R³⁵ and R³⁷ are selected from (1-6C)alkyl, (2-8C)alkenyl, (2-8C)alkynyl, (1-6C)alkylsulfonyl and (2-6C)alkanoyl,

or from a group of the formula:

—X¹⁰-Q⁷

wherein X¹⁰ is a direct bond or is selected from C(O), SO₂, wherein Q⁷ is (3-7C)cycloalkyl or (3-7C)cycloalkyl-(1-6C)alkyl;

R³⁶ is selected from halo, cyano, hydroxy, amino, (1-6C)alkyl, (2-6C)alkenyl, (2-6C)alkynyl, (3-6)cycloalkyl, (1-6C)alkoxy, (1-6C)alkylamino and di-[(1-6C)alkyl]amino;

or a pharmaceutically acceptable salt thereof.

In a particular embodiment, X^{a} in formula (I) above is oxygen. Thus examples of compound of formula (I) include compounds of formula (I′)

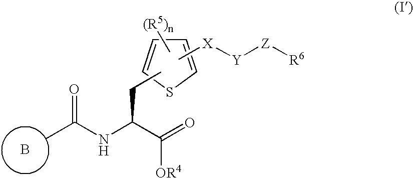

wherein R⁴, R⁵, R⁶, A, B, X, Y, Z and n are as defined above.

In an embodiment of the invention there is provided a compound of the formula I which is of the formula IA:

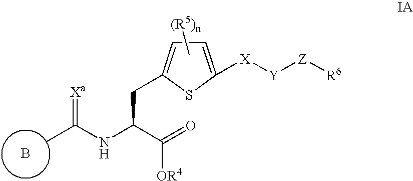

wherein:

n, B, R⁴, R⁵, R⁶, X^{a}, X, Y and Z are as hereinbefore defined;

or a pharmaceutically acceptable salt thereof. Particular compounds of the Formula IA are those wherein X^{a} is oxygen.

Other particular compounds of the formula (I), according to the invention, such as (IA) are those wherein the group R⁶-Z- in Formula I is not:

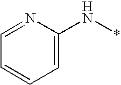

wherein * indicates the point of attachment of R⁶-Z- to the group Y in formula I.

In another embodiment of the invention there is provided a compound of the formula I of the formula (IB):

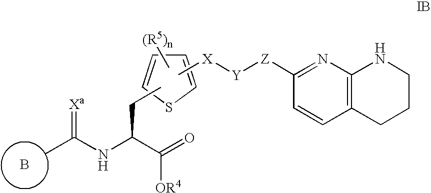

wherein:

n, B, R⁴, R⁵, X^{a}, X, Y and Z have any of the values as hereinbefore defined;

or a pharmaceutically acceptable salt thereof.

Particular compounds of the Formula IB are of the Formula IB′:

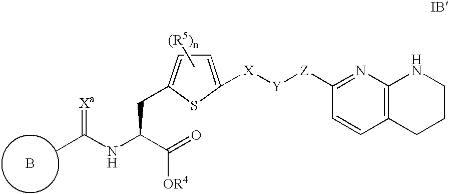

wherein:

n, B, R⁴, R⁵, X^{a}, X, Y and Z have any of the values as hereinbefore defined;

or a pharmaceutically acceptable salt thereof.

Particular compounds of the Formula IB and IB′ are those wherein X³ is oxygen.

In another embodiment of the invention there is provided a compound of the formula I of the formula (IC):

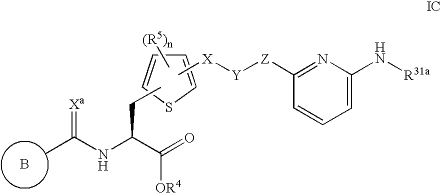

wherein:

n, B, R⁴, R⁵, R^{31a}, X^{a}, X, Y and Z have any of the values as hereinbefore defined;

or a pharmaceutically acceptable salt thereof.

Particular compounds of the Formula IC are of the Formula IC′:

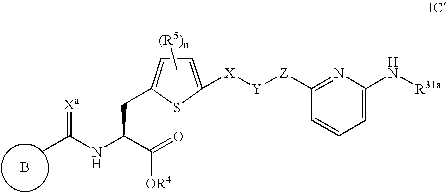

wherein:

n, B, R⁴, R⁵, R^{31a}, X^{a}, X, Y and Z have any of the values as hereinbefore defined;

or a pharmaceutically acceptable salt thereof.

Particular compounds of the Formula IC and IC′ are those wherein X³ is oxygen.

In another embodiment of the invention there is provided a compound of the formula I of the formula (ID):

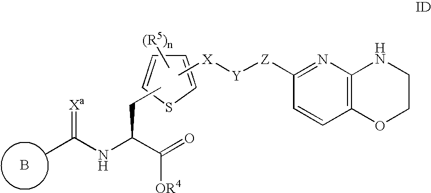

wherein:

n, B, R⁴, R⁵, X^{a}, X, Y and Z have any of the values as hereinbefore defined;

or a pharmaceutically acceptable salt thereof.

Particular compounds of the Formula ID are of the Formula ID′:

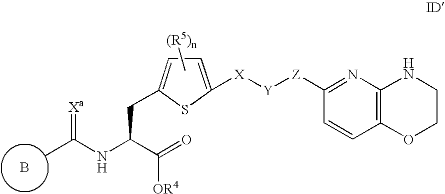

wherein:

n, B, R⁴, R⁵, X^{a}, X, Y and Z have any of the values as hereinbefore defined;

or a pharmaceutically acceptable salt thereof.

Particular compounds of the Formula ID and ID′ are those wherein X³ is oxygen.

Further particular compounds according to the invention are those of the Formula I, IA, IB, IB′, IC, IC′, ID and ID′ are those wherein the group —X— is a direct bond.

Further particular compounds according to the invention are those of the Formula I, IA, IB, IB′, IC, IC′, ID and ID′ are those wherein the group —X—Y-Z- is —(CH₂)₃—, and wherein —X—Y-Z- optionally bears on carbon one or more R²⁸ substituents, wherein R²⁸ is as hereinbefore defined. For example R²⁸ is (1-4C)alkyl, more particularly-X—Y-Z- is —(CH₂)₃—.

Further particular compounds according to the invention are those of the Formula I, IA, IB, IB′, IC, IC′, ID and ID′ are those wherein the group —X—Y-Z- is *-O(CH₂)₂—, wherein * indicates the point of attachment to the thienyl group and wherein —X—Y-Z- optionally bears on carbon one or more R²⁸ substituents, wherein R²⁸ is as hereinbefore defined. For example R²⁸ is (1-4C)alkyl, more particularly-X—Y-Z- is *-O(CH₂)₂—.

In one embodiment of the invention there is provided a compound of the formula I, or a pharmaceutically acceptable salt thereof, wherein R^{31a} is selected from R^{31a} is selected from hydrogen, (1-6C)alkyl, (2-8C)alkenyl, (2-8C)alkynyl, halo-(1-4C)alkyl, hydroxy-(2-4C)alkyl, (1-4C)alkoxy-(2-4C)alkyl, amino-(2-4C)alkyl, (1-4C)alkylamino-(2-4C)alkyl, di-[(1-4C)alkyl]amino-(2-4C)alkyl, (3-6C)cycloalkyl and (3-6C)cycloalkyl-(1-4C)alkyl, and wherein any (3-6C)cycloalkyl in R^{31a} optionally bears 1 or more (for example 1 or 2) (1-4C)alkyl substituents; and

B, X^{a}, R⁴, R⁵, R⁶, X, Y, Z and n are as hereinbefore defined.

Further particular compounds according to the invention are those of the Formula I, IA, IB, IB′, IC, IC′, ID and ID′ are those wherein R⁴ is hydrogen.

Examples of heteroaryl groups for B include aromatic mono and bicyclic heteroaryl rings containing from 5 to 12 atoms of which at least 1 atom (for example 1 to 5 atoms) is selected from oxygen, nitrogen and sulfur, for example, furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, indazolyl, purinyl, benzofurazanyl, quinolyl, isoquinolyl, quinazolinyl, quinoxalinyl, cinnolinyl, pteridinyl, naphthyridinyl, pyridopyrazinyl, thieno[2,3-b]furanyl, 2H-furo[3,2-b]-pyranyl, 5H-pyrido[2,3-d]-o-oxazinyl, 1H-pyrazolo[4,3-d]-oxazolyl, 4H-imidazo[4,5-d]thiazolyl, pyrazino[2,3-d]pyridazinyl, imidazo[2,1-b]thiazolyl, imidazo[1,2-b][1,2,4]triazinyl, 1,2,3,4-tetrahydro-1,8-naphthyridinyl, 1,2,3,4-tetrahydropyrido[2,3-b]pyrazinyl, 3,4-dihydro-2H-pyrido[3,2-b][1,4]oxazinyl indolizine, quinolizine and phthalazine.

For instance, B may be a heteroaromatic 5 or 6 membered monocyclic ring containing up to 4 atoms selected from oxygen nitrogen and sulfur, for example, furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazinyl. Particular examples are furyl, pyrrolyl, thienyl, oxazolyl, isoxazolyl, imidazolyl, pyrazolyl, thiazolyl and pyridyl.

Alternatively, B may be, for example, a heteroaromatic 9 or 10 membered bicyclic ring containing up to 5 atoms selected from oxygen, nitrogen and sulfur, for example, benzofuranyl, indolyl, isoindolyl, benzothienyl, benzoxazolyl, benzimidazolyl, benzothiazolyl, indazolyl, purinyl, benzofurazanyl, quinolyl, isoquinolyl, quinazolinyl, quinoxalinyl, cinnolinyl, pteridinyl, naphthyridinyl, pyridopyrazinyl, thieno[2,3-b]furanyl, 2H-furo[3,2-b]-pyranyl, 5H-pyrido[2,3-d]-o-oxazinyl, 1H-pyrazolo[4,3-d]-oxazolyl, 4H-imidazo[4,5-d]thiazolyl, pyrazino[2,3-d]pyridazinyl, imidazo[2,1-b]thiazolyl, imidazo[1,2-b][1,2,4]triazinyl, 1,2,3,4-tetrahydro-1,8-naphthyridinyl, 1,2,3,4-tetrahydropyrido[2,3-b]pyrazinyl and 3,4-dihydro-2H-pyrido[3,2-b][1,4]oxazinyl.

In one embodiment, B is a fully aromatic bicyclic ring such as benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, pyridopyrazinyl, thieno[2,3-b]furanyl, 2H-furo[3,2-b]-pyranyl, 5H-pyrido[2,3-d]-o-oxazinyl, 1H-pyrazolo[4,3-d]-oxazolyl, 4H-imidazo[4,5-d]thiazolyl, pyrazino[2,3-d]pyridazinyl, imidazo[2,1-b]thiazolyl, imidazo[1,2-b][1,2,4]triazinyl. An example of such a ring is benzofuranyl or indolyl.

As hereinbefore defined the ring B is substituted ortho to the C(X^{a}) group in formula (I) by R¹ and optionally bears one or more R³, for example from 1 to 5 R³ substituents, and in particular 1, 2 or 3 R³ substituents from those listed above.

The substituent(s) on ring B may be attached to any carbon or nitrogen atom within the ring B, provided that, in the case of substitution on a nitrogen atom, the aromaticity of the ring is not eliminated.

In one embodiment of the invention ring B is substituted by R¹ in an ortho position to the C(X^{a}) group in formula (I) and optionally bears 1 to 3 additional R³ substituents, wherein R¹ and R³ on ring B are as hereinbefore defined.

In another embodiment of the invention ring B is substituted by R¹ in an ortho position to the C(X³) group in formula (I) and optionally bears 1 to 3 additional R³ substituents, wherein R¹ is selected from halo (for example fluoro, chloro or bromo, particularly bromo or chloro), (1-3C)alkyl, cyclopropyl, cyclobutyl, cyclopentyl, cyclopropylmethyl, cyclobutlymethyl, cyclopentylmethyl and halo-(1-3C)alkyl; and R³ is as hereinbefore defined.

In another embodiment of the invention ring B is substituted by R¹ in one ortho position to the C(X³) group in formula (I) and is optionally substituted in the other ortho position to the C(X³) group by an R³ group which is R³, and wherein ring B optionally bears 1 to 3 additional R³ substituents, wherein:

R¹ and R^{3′}, which may be the same or different, are selected from halo (for example fluoro, chloro or bromo, more particularly chloro or bromo), (1-3C)alkyl, cyclopropyl, cyclobutyl, cyclopentyl, cyclopropylmethyl, cyclobutlymethyl, cyclopentylmethyl, (1-3C)alkoxy, (1-3C)alkylthio and halo-(1-3C)alkyl; and

R³ is as hereinbefore defined.

Suitably in this embodiment R¹ and R³, which may be the same or different, are selected from fluoro, chloro, bromo, (1-3C)alkyl, (1-3C)alkoxy and (1-3C)alkylthio (for example R¹ and R³ are (1-3C)alkyl such as methyl or ethyl);

R³ is as hereinbefore defined for a substituent that may be present on ring B, for example R³ is selected from fluoro, chloro, bromo, (1-3C)alkyl, (1-3C)alkoxy, (1-3C)alkylthio and halo-(1-3C)alkyl.

Suitably in this embodiment ring B is substituted by R¹ in one ortho position to the C(X^{a}) group and is also substituted by R^{3′} in the other ortho position to the C(X^{a}) group in formula I, and wherein ring B optionally bears 1 to 3 additional R³ substituents.

In another embodiment of the invention ring B is substituted in one ortho position to the C(X^{a}) group in formula (I) by R¹ and is optionally substituted in the other ortho position to the C(X³) group by an R³ group which is R³; and wherein ring B optionally bears 1 to 3 additional R³ substituents, wherein:

R¹ is selected from chloro, bromo, (1-3C)alkyl, cyclopropyl, cyclobutyl, cyclopentyl, cyclopropylmethyl, cyclobutlymethyl, cyclopentylmethyl and halo-(1-3C)alkyl;

R^{3′} is selected from fluoro, chloro, bromo, (1-3C)alkyl and halo-(1-3C)alkyl; and

R³ is as hereinbefore defined.

In another embodiment of the invention ring B is substituted by R¹ and an R³ group which is R^{3′} in the ortho positions to the C(X^{a}) group in formula (I), and wherein ring B optionally bears 1 to 3 additional R³ substituents, wherein:

R¹ is selected from chloro, bromo and (1-3C)alkyl;

R^{3′} is selected from fluoro, chloro, bromo and (1-3C)alkyl; and

R³ is as hereinbefore defined.

In another embodiment of the invention ring B is substituted by R¹ and an R³ group which is R³ in the ortho positions to the C(X^{a}) group in formula (I) and wherein ring B optionally bears 1 to 3 additional R³ substituents, wherein:

R¹ and R^{3′}, which may be the same or different are both halo (for example fluoro, chloro or bromo, particularly chloro or bromo, more particularly chloro); and

R³ is as hereinbefore defined.

In another embodiment of the invention ring B is substituted by R¹ and an R³ group which is R³ in the ortho positions to the C(X^{a}) group in formula (I) and wherein ring B optionally bears 1 to 3 additional R³ substituents, wherein:

R¹ and R^{3′}, which may be the same or different are both (1-3C)alkyl (for example methyl or ethyl, more particularly methyl); and

R³ is as hereinbefore defined.

Particular examples of the group:

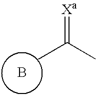

in formulae (I), (I′) and (IA) are groups of sub-formula (a) or (b):

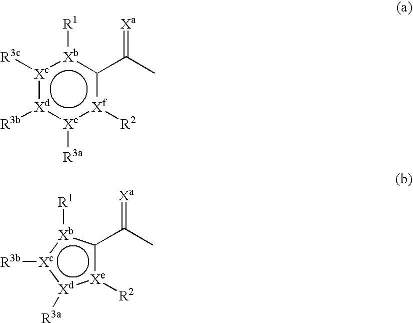

wherein X^{a} is as defined above, X^{b} is carbon or nitrogen, X^{c}, X^{d}, X^{e} and X^{f} are independently selected from carbon, nitrogen, oxygen or sulphur, provided that (i) at least one of X^{b}, X^{c}, X^{d}, X^{e} and X^{f} is selected from nitrogen oxygen and sulphur, (ii) at least one and in particular at least two of X^{b}, X^{c}, X^{d}, X^{e} and X^{f} are carbon and (iii) there are no O—O, O—S or S—S bonds within the ring;

R¹ is a substituent for the B ring as listed above, R², R^{3a}, R^{3b} and R^{3c} are independently selected from hydrogen, a substituent for the B ring as listed above in relation to R³, or are absent where the group X^{b}, X^{c}, X^{d}, X^{e} or X^{f} to which they are attached is an oxygen or sulphur atom, or two adjacent groups R¹, R², R^{3a}, R^{3b} or R^{3c} are joined to form a fused 5-7 membered ring which optionally contains additional heteroatoms selected from nitrogen, oxygen and sulphur and may carry further substituents as defined above for the ring B, provided that where any of X^{b}, X^{c}, X^{d}, X^{e} and X^{f} are nitrogen, then the group R¹, R^{3c}, R^{3b}, R^{3a} and R² to which they are attached are other than halo, cyano, nitro, hydroxy, mercapto or any other group which cannot stably bind to a nitrogen atom.

In a particular embodiment, the ring B carries a substituent at a position which is ortho to the link to the C(X^{a}) group in formula (I). Thus, X^{b} is carbon or, where the ring aromaticity allows, nitrogen, and R¹ is other than hydrogen.

In one embodiment, R¹ and optionally also one or two of R^{3a}, R^{3b}, R^{c} and R² are selected from halo, trifluoromethyl, cyano, isocyano, nitro, hydroxy, mercapto, amino, formyl, carboxy, carbamoyl, sulfamoyl, halo-(1-3C)alkyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

or from a group of the formula:

Q¹-X¹—

wherein X¹ is a direct bond or is selected from O, S, SO, SO₂, N(R⁷), C(O), CH(OR⁷), C(O)N(R⁷), N(R⁷)C(O), SO₂N(R⁷), N(R⁷)SO₂, OC(R⁷)₂, SC(R⁷)₂ and N(R⁷)C(R⁷)₂, wherein R⁷ is hydrogen or (1-6C)alkyl, and Q¹ is aryl, aryl-(1-6C)alkyl, (3-7C)cycloalkyl, (3-7C)cycloalkyl-(1-6C)alkyl, (3-7C)cycloalkenyl, (3-7C)cycloalkenyl-(1-6C)alkyl, heteroaryl, heteroaryl-(1-6C)alkyl, heterocyclyl or heterocyclyl-(1-6C)alkyl,

and wherein any carbon containing substituent on ring B optionally bears on carbon one or more R⁸ groups,

and wherein if any heteroaryl or heterocyclyl group which is a substituent on ring B contains an —NH— moiety, the nitrogen of said moiety optionally bears a group selected from R⁹,

and wherein any heterocyclyl group which is a substituent on ring B optionally bears 1 or 2 oxo or thioxo substituents;

or two adjacent substituents on ring B optionally form a (1-3C)alkylenedioxy group;

and wherein ring B is linked to the C(X^{a}) group by a carbon atom.

Particular examples of R¹ include halo, trifluoromethyl, cyano, isocyano, nitro, hydroxy, mercapto, amino, formyl, carboxy, carbamoyl, sulfamoyl, halo-(1-3C)alkyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

or from a group of the formula:

Q¹-X¹—

wherein X¹ is a direct bond or is selected from O, S, SO, SO₂, N(R⁷), C(O), CH(OR⁷), C(O)N(R⁷), N(R⁷)C(O), SO₂N(R⁷), N(R⁷)SO₂, OC(R⁷)₂, SC(R⁷)₂ and N(R⁷)C(R⁷)₂, wherein R⁷ is hydrogen or (1-6C)alkyl, and Q¹ is aryl, aryl-(1-6C)alkyl, (3-7C)cycloalkyl, (3-7C)cycloalkyl-(1-6C)alkyl, (3-7C)cycloalkenyl, (3-7C)cycloalkenyl-(1-6C)alkyl, heteroaryl, heteroaryl-(1-6C)alkyl, heterocyclyl or heterocyclyl-(1-6C)alkyl.

In particular, R¹ is selected from halo (particularly fluoro or chloro, more particularly chloro), (1-3C)alkyl, (1-3C)alkoxy, (1-3C)thioalkyl and halo-(1-3C)alkyl. More particularly R¹ is selected from chloro and (1-3C)alkyl, still more particularly R¹ is chloro.

R², R^{1a}, R^{3b} and R^{3c} which may be the same or different, are selected from hydrogen, halo, trifluoromethyl, cyano, isocyano, nitro, hydroxy, mercapto, amino, formyl, carboxy, carbamoyl, sulfamoyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

or from a group of the formula:

Q¹-X¹—

wherein X¹ is a direct bond or is selected from O, S, SO, SO₂, N(R⁷), C(O), CH(OR⁷), C(O)N(R⁷), N(R⁷)C(O), SO₂N(R⁷), N(R⁷)SO₂, OC(R⁷)₂, SC(R⁷)₂ and N(R⁷)C(R⁷)₂,

wherein R⁷ is hydrogen or (1-6C)alkyl, and Q¹ is aryl, aryl-(1-6C)alkyl, (3-7C)cycloalkyl, (3-7C)cycloalkyl-(1-6C)alkyl, (3-7C)cycloalkenyl, (3-7C)cycloalkenyl-(1-6C)alkyl, heteroaryl, heteroaryl-(1-6C)alkyl, heterocyclyl or heterocyclyl-(1-6C)alkyl,

and wherein R², R^{3a}, R^{3b} or R^{3c} independently of each other optionally bears on carbon one or more R⁸ groups,

and wherein if any heteroaryl or heterocyclyl group within R², R^{3a}, R^{3b} or R^{3c} contains an —NH— moiety, the nitrogen of said moiety optionally bears a group selected from R⁹,

and wherein any heterocyclyl group within R², R^{3a}, R^{3b} or R^{3c} optionally bears 1 or 2 oxo or thioxo substituents;

or any adjacent two of R², R^{3a}, R^{3b} or R^{3c} optionally form a (1-3C)alkylenedioxy group.

In a particular embodiment however, R², R^{3a}, R^{3b} and R^{3c} are independently selected from hydrogen, halo (particularly fluoro or chloro, more particularly chloro), (1-3C)alkyl, (1-3C)alkoxy, (1-3C)thioalkyl and halo-(1-3C)alkyl.

In another embodiment X^{b} is carbon and R¹ is selected from halo (particularly fluoro or chloro, more particularly chloro) and (1-3C)alkyl (particularly methyl), or X^{b} is nitrogen and R¹ is (1-3C)alkyl (particularly methyl); and when any of R², R^{3a}, R^{3b} and R^{3c} are attached to carbon they are each independently selected from hydrogen, halo (particularly fluoro or chloro, more particularly chloro), (1-3C)alkyl, (1-3C)alkoxy, (1-3C)thioalkyl and halo-(1-3C)alkyl; and when any of and when any of R², R^{3a}, R^{3b} and R^{3c} are attached to nitrogen they are each independently selected from hydrogen and (1-3C)alkyl.

In another embodiment of the invention ring B is pyridyl, particularly 4-pyridyl, and wherein ring B is substituted in an ortho position to the C(X^{a}) group in formula I by R¹ and optionally bears one or more R³ substituents (for example 1, 2 or 3 substituents), wherein R¹ and each R³ have any of the values defined hereinbefore or hereinafter.

More particularly, ring B is pyridyl, particularly 4-pyridyl, and wherein ring B is substituted in an ortho position to the C(X^{a}) group in formula I by R¹ and is substituted in the other ortho position to the C(X^{a}) group by R², and wherein ring B optionally bears 1 or 2 additional R³ substituents; wherein R¹ and R³ are as hereinbefore defined and R² is hydrogen or any of the values defined herein for R³. Suitably in this embodiment R² is not hydrogen.

In a particular embodiment of the invention ring B is of the formula B1:

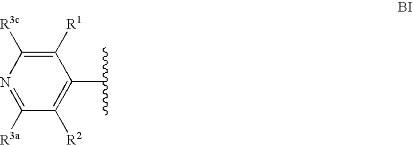

wherein R¹ is as hereinbefore defined and R², R^{3a} and R^{3c}, which may be the same or different, are hydrogen or any of the values defined herein for R³. In one embodiment R² is hydrogen in ring BI. In another embodiment in ring BI R² is as hereinbefore defined for R³. Particularly in ring BI, R¹ is selected from fluoro, chloro, bromo, methyl, ethyl, methylthio, ethylthio, methoxy and ethoxy; R² is selected from hydrogen, methyl, ethyl, fluoro, chloro and bromo (suitably R² is not hydrogen) and R^{3a} and R^{3c}, which may be the same or different, are hydrogen or have any of the values defined herein for R³. In a still further embodiment R¹ and R² are selected from chloro and methyl (particularly R¹ and R² are both chloro) and R^{3a} and R^{3c}, which may be the same or different, are hydrogen or have any of the values defined herein for R³, for example R^{3a} and R^{3c} are selected from hydrogen, halo (particularly fluoro or chloro, more particularly chloro), (1-3C)alkyl, (1-3C)alkoxy, (1-3C)thioalkyl and trifluoromethyl). For example R^{3a} and R^{3c} are both hydrogen.

In another embodiment of the invention ring B is isoxazolyl, particularly isoxazol-4-yl, and wherein the isoxazolyl representing ring B is substituted in an ortho position to the C(X^{a}) group in formula I by R¹ and optionally bears one or more R³ substituents (for example 1, 2 or 3 substituents), wherein R¹ and each R³ have any of the values defined hereinbefore or hereinafter. For example, ring B is of the formula BII:

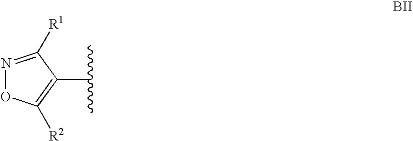

wherein R¹ is as hereinbefore defined and R² is hydrogen or any of the values defined herein for R³.

In one embodiment R² is hydrogen in ring BII. In another embodiment in ring BII R² is as hereinbefore defined for R³.

In another embodiment in ring BII:

R¹ is selected from halo, (1-3C)alkyl, (1-3C)alkoxy, (1-3C)alkylthio, cyclopropyl, cyclobutyl, cyclopentyl, cyclopropyl-(1-2C)alkyl, cyclobutyl-(1-2C)alkyl and cyclopentyl-(1-2C)alkyl (particularly R¹ is selected from halo and (1-3C)alkyl such as fluoro, chloro, bromo, methyl and ethyl more particularly R¹ is selected from chloro and methyl); and

R² is selected from hydrogen, halo, (1-3C)alkyl, (1-3C)alkoxy, (1-3C)alkylthio, halo-(1-3C)alkyl, cyclopropyl, cyclobutyl, cyclopentyl, cyclopropyl-(1-2C)alkyl, cyclobutyl-(1-2C)alkyl and cyclopentyl-(1-2C)alkyl (for example R² is selected from hydrogen, fluoro, chloro, bromo, methyl, ethyl and trifluoromethyl; more particularly R² is selected from hydrogen, fluoro, chloro, bromo, methyl and ethyl; still more particularly R² is selected from chloro, bromo, methyl and ethyl). Suitably in this embodiment R² is not hydrogen.

In another embodiment in ring BII, R¹ is (1-3C) alkyl and R² is hydrogen or (1-3C)alkyl. Particularly R¹ and R² are both (1-3C)alkyl such as methyl or ethyl. Accordingly a particular ring B is 3,5-dimethylisoxazol-4-yl.

Also provided is a compound of formula I, or a pharmaceutically acceptable salt or prodrug thereof in association with a pharmaceutically acceptable carrier, diluent, or excipient.

Also provided is a compound of formula I, or a pharmaceutically acceptable salt or prodrug thereof, which is an integrin inhibitor useful for controlling pathologically angiogenic diseases, thrombosis, cardiac infarction, coronary heart diseases, arteriosclerosis, tumours, osteoporosis, inflammations or infections.

Also provided is a method of treating a disease or condition mediated by a5b1 which comprises administering to a patient in need of such treatment a compound of formula I, or a pharmaceutically acceptable salt or prodrug thereof.

Also provided is a process for the preparation of a compound of formula I as defined herein.

### DETAILED DESCRIPTION OF THE INVENTION

Unless otherwise stated, the following terms used in the specification and claims have the following meanings.

### Definitions

“Halo” means fluoro, chloro, bromo or iodo.

“(1-6C)alkyl” means a linear saturated monovalent hydrocarbon radical of one to six carbon atoms or a branched saturated monovalent hydrocarbon radical of three to six carbon atoms, for example methyl, ethyl, propyl, 2-propyl, tert-butyl, sec-butyl, n-pentyl, n-hexyl and the like.

An “alkylene,” “alkenylene,” or “alkynylene” group is an alkyl, alkenyl, or alkynyl group that is positioned between and serves to connect two other chemical groups. Thus, “(1-6C)alkylene” means a linear saturated divalent hydrocarbon radical of one to six carbon atoms or a branched saturated divalent hydrocarbon radical of three to six carbon atoms, for example, methylene, ethylene, propylene, 2-methylpropylene, pentylene, and the like.

“(2-6C)Alkenylene” means a linear divalent hydrocarbon radical of two to six carbon atoms or a branched divalent hydrocarbon radical of three to six carbon atoms, containing at least one double bond, for example, as in ethenylene, 2,4-pentadienylene, and the like. For the avoidance of doubt however, the term “cycloalkenylene” refers to unsaturated carbocyclic rings, which are not aromatic in nature, and therefore this expression does not include aryl groups.

“(2-6C)Alkynylene” means a linear divalent hydrocarbon radical of two to six carbon atoms or a branched divalent hydrocarbon radical of three to six carbon atoms, containing at least one triple bond, for example, as in ethynylene, propynylene, and butynylene and the like.

“(3-7C)Cycloalkyl” means a hydrocarbon ring containing from 3 to 7 carbon atoms, for example, cyclopropyl, cyclobutyl, cyclopentyl, cyclohexyl or bicyclo[2.2.1]heptyl

“(3-7C)Cycloalkenyl” means a hydrocarbon ring containing at least one double bond, for example, cyclobutenyl, cyclopentenyl, cyclohexenyl or cycloheptenyl, such as 3-cyclohexen-1-yl.

“(3-7C)Cycloalkyl-(1-6C)alkylene” means a (3-7C)cycloalkyl group covalently attached to a (1-6C)alkylene group, both of which are defined herein, for example cyclopropylmethyl, 2-cyclopropylethyl, 1-cyclopropylethyl and cyclohexylmethyl.

The term “non-aromatic” refers to unsaturated ring systems without aromatic character, for example partially saturated and fully saturated carbocyclic and heterocyclic ring systems. The terms “unsaturated” and “partially saturated” refer to non-aromatic rings wherein the ring structure(s) contains atoms sharing more than one valence bond i.e. the ring contains at least one multiple bond e.g. a C═C, C≡C or N═C bond. The term “fully saturated” refers to rings where there are no multiple bonds between ring atoms. Saturated carbocyclic groups include, for example the cycloalkyl groups as defined herein. Partially saturated carbocyclic groups include cycloalkenyl groups as defined herein, for example cyclopentenyl, cycloheptenyl and cyclooctenyl. Partially saturated heterocyclyl rings include for example, dihydrothiophene, dihydrofuran, pyrroline, dihydropyran or tetrahydropyridine.

The term “heterocyclyl” or “heterocyclic” means a non-aromatic saturated or partially saturated monocyclic, fused, bridged, or spiro bicyclic heterocyclic ring system(s). Monocyclic heterocyclic rings contain from about 3 to 12 ring atoms, with from 1 to 5 heteroatoms selected from N, O, and S, and suitably from 3 to 7 member atoms, in the ring. Bicyclic heterocycles contain from 7 to 17 member atoms, suitably 7 to 12 member atoms, in the ring. Bicyclic heterocycles contain from about 7 to about 17 ring atoms, suitably from 7 to 12 ring atoms. Bicyclic heterocyclic(s) rings may be fused, spiro, or bridged ring systems. Examples of heterocyclic groups include cyclic ethers (oxiranes) such as ethyleneoxide, tetrahydrofuran, dioxane, and substituted cyclic ethers. Heterocycles containing nitrogen include, for example, azetidine, pyrrolidine, piperidine, piperazine, tetrahydrotriazine, tetrahydropyrazole, and the like. Typical sulfur containing heterocycles include tetrahydrothiophene, dihydro-1,3-dithiol-2-yl, and hexahydrothiepin-4-yl. Other heterocycles include dihydro-oxathiol-4-yl, tetrahydro-oxazolyl, tetrahydro-oxadiazolyl, tetrahydrodioxazolyl, tetrahydro-oxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothiophene. A suitable value for a heterocyclyl group which bears 1 or 2 oxo or thioxo substituents is, for example, 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl.

By “bridged ring systems” is meant ring systems in which two rings share more than two atoms, see for example Advanced Organic Chemistry, by Jerry March, 4^{th} Edition, Wiley Interscience, pages 131-133, 1992. Examples of bridged heterocyclyl ring systems include, aza-bicyclo[2.2.1]heptane, 2-oxa-5-azabicyclo[2.2.1]heptane, aza-bicyclo[2.2.2]octane, and aza-bicyclo[3.2.1]octane.

“Heterocyclyl-(1-6C)alkyl” means a heterocyclyl group covalently attached to a (1-6C)alkylene group, both of which are defined herein.

The term “aryl” means a cyclic or polycyclic aromatic ring having from 5 to 12 carbon atoms and no ring heteroatoms. The term aryl includes both monovalent species and divalent species. Examples of aryl groups include, but are not limited to, phenyl, biphenyl, naphthyl and the like. The term “aryl” also embraces polycyclic carbon ring systems wherein one or more rings are non-aromatic, provided that at least one ring is aromatic and said rings do not contain ring heteroatoms. In such polycyclic aryl ring systems unless specified otherwise herein, the group may be attached by the aromatic ring, or by a non-aromatic ring. Examples of bicyclic aryl groups containing an aromatic ring and a non-aromatic ring include tetrahydronaphthalene and indane groups.

“Aryl-(1-6C)alkyl” means an aryl group covalently attached to a (1-6C)alkyl group, both of which are defined herein. Examples of aryl-(1-6C)alkyl groups include benzyl, phenylethyl, and the like

The term “heteroaryl” means an aromatic mono-, bi-, or polycyclic ring incorporating one or more heteroatoms selected from N, O, and S. The term heteroaryl includes both monovalent species and divalent species. Examples of heteroaryl groups are monocyclic and bicyclic groups containing from five to twelve ring members, and more usually from five to ten ring members. The heteroaryl group can be, for example, a five membered or six membered monocyclic ring or a bicyclic structure formed from fused five and six membered rings or two fused six membered rings. Each ring may contain up to about four heteroatoms typically selected from nitrogen, sulphur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 5H-pyrido[2,3-d]-o-oxazinyl, 1H-pyrazolo[4,3-d]-oxazolyl, 4H-imidazo[4,5-d]thiazolyl, pyrazino[2,3-d]pyridazinyl, imidazo[2,1-b]thiazolyl, imidazo[1,2-b][1,2,4]triazinyl. “Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from O, S and N. Examples of partially aromatic heteroaryl groups include for example, tetrahydroisoquinoline, tetrahydroquinoline, dihydrobenzthiene, dihydrobenzfuran, 2,3-dihydro-benzo[1,4]dioxine, benzo[1,3]dioxole, 4,5,6,7-tetrahydrobenzofuran, indoline, 1,2,3,4-tetrahydro-1,8-naphthyridinyl, 1,2,3,4-tetrahydropyrido[2,3-b]pyrazinyl and 3,4-dihydro-2H-pyrido[3,2-b][1,4]oxazinyl. References herein to a “6,5” or “6,6” aryl or heteroaryl ring systems refer to 5 membered ring fused to another 6 membered ring such as a benzothienyl ring (a 6,5 ring); or one 6 membered ring fused to another 6 membered ring such as a napthyl, quinolyl or quinazolinyl ring (a 6,6 ring). Unless stated otherwise, a 6,5 heteroaryl group may be attached via the 5 or the 6 membered ring.

Examples of five membered heteroaryl groups include but are not limited to pyrrole, furan, thiophene, imidazole, furazan, oxazole, oxadiazole, oxatriazole, isoxazole, thiazole, isothiazole, pyrazole, triazole and tetrazole groups.

Examples of six membered heteroaryl groups include but are not limited to pyridine, pyrazine, pyridazine, pyrimidine and triazine.

A bicyclic heteroaryl group may be, for example, a group selected from:
a) a benzene ring fused to a 5- or 6-membered ring containing 1, 2 or 3 ring heteroatoms;
b) a pyridine ring fused to a 5- or 6-membered ring containing 1, 2 or 3 ring heteroatoms;
c) a pyrimidine ring fused to a 5- or 6-membered ring containing 1 or 2 ring heteroatoms;
d) a pyrrole ring fused to a 5- or 6-membered ring containing 1, 2 or 3 ring heteroatoms;
e) a pyrazole ring fused to a 5- or 6-membered ring containing 1 or 2 ring heteroatoms;
f) a pyrazine ring fused to a 5- or 6-membered ring containing 1 or 2 ring heteroatoms;
g) an imidazole ring fused to a 5- or 6-membered ring containing 1 or 2 ring heteroatoms;
h) an oxazole ring fused to a 5- or 6-membered ring containing 1 or 2 ring heteroatoms;
i) an isoxazole ring fused to a 5- or 6-membered ring containing 1 or 2 ring heteroatoms;
j) a thiazole ring fused to a 5- or 6-membered ring containing 1 or 2 ring heteroatoms;
k) an isothiazole ring fused to a 5- or 6-membered ring containing 1 or 2 ring heteroatoms;
l) a thiophene ring fused to a 5- or 6-membered ring containing 1, 2 or 3 ring heteroatoms;
m) a furan ring fused to a 5- or 6-membered ring containing 1, 2 or 3 ring heteroatoms;
n) a cyclohexyl ring fused to a 5- or 6-membered heteroaromatic ring containing 1, 2 or 3 ring heteroatoms; and
o) a cyclopentyl ring fused to a 5- or 6-membered heteroaromatic ring containing 1, 2 or 3 ring heteroatoms.

Particular examples of bicyclic heteroaryl groups containing a six membered ring fused to a five membered ring include but are not limited to benzfuran, benzthiophene, benzimidazole, benzoxazole, benzisoxazole, benzthiazole, benzisothiazole, isobenzofuran, indole, isoindole, indolizine, indoline, isoindoline, purine (e.g., adenine, guanine), indazole, benzodioxole and pyrazolopyridine groups.

Particular examples of bicyclic heteroaryl groups containing two fused six membered rings include but are not limited to quinoline, isoquinoline, chroman, thiochroman, chromene, isochromene, chroman, isochroman, benzodioxan, quinolizine, benzoxazine, benzodiazine, pyridopyridine, quinoxaline, quinazoline, cinnoline, phthalazine, naphthyridine and pteridine groups.

“Heteroaryl-(1-6C)alkyl” means an heteroaryl group covalently attached to a (1-6C)alkyl group, both of which are defined herein. Examples of heteroaralkyl groups include pyridin-3-ylmethyl, 3-(benzofuran-2-yl)propyl, and the like.

“Haloalkyl” means alkyl substituted with one or more same or different halo atoms, e.g., —CH₂Cl, —CF₃, —CH₂CF₃, —CH₂CCl₃, and the like.

Examples for the substituents within the compound of formula I include:—
for halo fluoro, chloro, bromo and iodo;
for (1-6C)alkyl: methyl, ethyl, propyl, isopropyl and tert-butyl;
for (2-8C)alkenyl: vinyl, isopropenyl, allyl and but-2-enyl;
for (2-8C)alkynyl: ethynyl, 2-propynyl and but-2-ynyl;
for (1-6C)alkoxy: methoxy, ethoxy, propoxy, isopropoxy and butoxy;
for (2-6C)alkenyloxy: vinyloxy and allyloxy;
for (2-6C)alkynyloxy: ethynyloxy and 2-propynyloxy;
for (1-6C)alkylthio: methylthio, ethylthio and propylthio;
for (1-6C)alkylsulfinyl: methylsulfinyl and ethylsulfinyl;
for (1-6C)alkylsulfonyl: methylsulfonyl and ethylsulfonyl;
for (1-6C)alkylamino: methylamino, ethylamino, propylamino, isopropylamino and butylamino;
for di-[(1-6C)alkyl]amino: dimethylamino, diethylamino, N-ethyl-N-methylamino and diisopropylamino;
for (1-6C)alkoxycarbonyl: methoxycarbonyl, ethoxycarbonyl, propoxycarbonyl and tert-butoxycarbonyl;
for N-(1-6C)alkylcarbamoyl: N-methylcarbamoyl, N-ethylcarbamoyl and N-propylcarbamoyl;
for N,N-di-[(1-6C)alkyl]carbamoyl: N,N-dimethylcarbamoyl, N-ethyl-N-methylcarbamoyl and N,N-diethylcarbamoyl;
for (2-6C)alkanoyl: acetyl and propionyl;
for (2-6C)alkanoyloxy: acetoxy and propionyloxy;
for (2-6C)alkanoylamino: acetamido and propionamido;
for N-(1-6C)alkyl-(2-6C)alkanoylamino: N-methylacetamido and N-methylpropionamido;
for N-(1-6C)alkylsulfamoyl: N-methylsulfamoyl and N-ethylsulfamoyl;
for N,N-di-[(1-6C)alkyl]sulfamoyl: N,N-dimethylsulfamoyl;
for (1-6C)alkanesulfonylamino: methanesulfonylamino and ethanesulfonylamino;
for N-(1-6C)alkyl-(1-6C)alkanesulfonylamino: N-methylmethanesulfonylamino and N-methylethanesulfonylamino;
for (3-6C)alkenoylamino: acrylamido, methacrylamido and crotonamido;
for N-(1-6C)alkyl-(3-6C)alkenoylamino: N-methylacrylamido and N-methylcrotonamido;
for (3-6C)alkynoylamino: propiolamido;
for N-(1-6C)alkyl-(3-6C)alkynoylamino: N-methylpropiolamido;
for amino-(1-6C)alkyl: aminomethyl, 2-aminoethyl, 1-aminoethyl and 3-aminopropyl;
for (1-6C)alkylamino-(1-6C)alkyl: methylaminomethyl, ethylaminomethyl, 1-methylaminoethyl, 2-methylaminoethyl, 2-ethylaminoethyl and 3-methylaminopropyl;
for di-[(1-6C)alkyl]amino-(1-6C)alkyl: dimethylaminomethyl, diethylaminomethyl, 1-dimethylaminoethyl, 2-dimethylaminoethyl and 3-dimethylaminopropyl;
for hydroxy-(1-6C)alkyl: hydroxymethyl, 2-hydroxyethyl, 1-hydroxyethyl and 3-hydroxypropyl;
for (1-6C)alkoxy-(1-6C)alkyl: methoxymethyl, ethoxymethyl, 1-methoxyethyl, 2-methoxyethyl, 2-ethoxyethyl and 3-methoxypropyl;
for cyano-(1-6C)alkyl: cyanomethyl, 2-cyanoethyl, 1-cyanoethyl and 3-cyanopropyl;
for (1-6C)alkylthio-(1-6C)alkyl: methylthiomethyl, ethylthiomethyl, 2-methylthioethyl, 1-methylthioethyl and 3-methylthiopropyl;
for (1-6C)alkylsulfinyl-(1-6C)alkyl: methylsulfinylmethyl, ethylsulfinylmethyl, 2-methylsulfinylethyl, 1-methylsulfinylethyl and 3-methylsulfinylpropyl;
for (1-6C)alkylsulfonyl-(1-6C)alkyl: methylsulfonylmethyl, ethylsulfonylmethyl, 2-methylsulfonylethyl, 1-methylsulfonylethyl and 3-methylsulfonylpropyl;
for (2-6C)alkanoylamino-(1-6C)alkyl: acetamidomethyl, propionamidomethyl and 2-acetamidoethyl; and
for (1-6C)alkoxycarbonylamino-(1-6C)alkyl: methoxycarbonylaminomethyl, ethoxycarbonylaminomethyl, tert-butoxycarbonylaminomethyl and 2-methoxycarbonylaminoethyl.

The term “(1-3C)alkylenedioxy” includes for example, methylenedioxy or ethylenedioxy and the oxygen atoms thereof occupy adjacent ring positions.

When, as defined hereinbefore, ring B carries a substituent of the formula Q¹-X¹— and, for example, X¹ is a OC(R⁷)₂ linking group, it is the carbon atom, not the oxygen atom, of the OC(R⁷)₂ linking group which is attached to ring B in formula I and the oxygen atom is attached to the Q¹ group. Similarly, when, for example R⁹ is a group of the formula —X⁴—R¹⁵ and, for example, X⁴ is a C(O)N(R¹⁶) linking group, it is the N(R¹⁶) group, not the carbonyl group of the C(O)N(R¹⁶) linking group which is attached to the R¹⁵ group. A similar convention applies to the attachment of the groups of the formulae “Q-X—” and “R—X—” defined herein.

As defined hereinbefore, adjacent carbon atoms in any (2-6C)alkylene chain within for example an X, Y or Z group may be optionally separated by the insertion into the chain of a group such as O, C(O)N(R²⁶) or C≡C. For example, insertion of a C≡C group into the ethylene chain gives a but-2-ynylene group and, for example, insertion of a C(O)NH group into an ethylene chain gives rise to —CH₂C(O)NHCH₂—, similarly the insertion of an oxygen atom into a propylene chain gives for example —CH₂OCH₂CH₂—.

Where herein it is stated that the chain length of the group —X—Y-Z- is, for example 3 atoms, this means that the number of linked atoms between the thienyl ring and R⁶ is 3. For example, where —X—Y-Z- is:

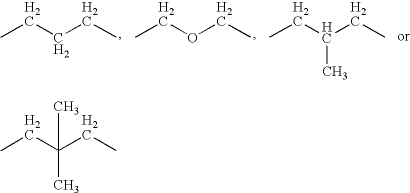

the chain length of —X—Y-Z- is 3 atoms.

Similarly, references herein to the chain length between the thienyl ring and a —N═ atom in R⁶, refer to the shortest chain length of the group —X—Y-Z- together with any ring atoms (including bridgehead atoms) in R⁶ up to and including said —N═ ring atom. By way of example, the chain length between the thienyl ring and the —N═ ring atom in R⁶ in each —X—Y-Z-R⁶ group shown below is 5:

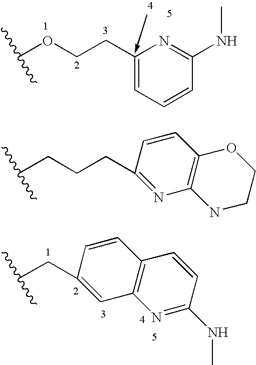

wherein [symbol] indicates the point of attachment of —X—Y-Z-R⁶ to the thienyl ring in formula I.

Reference herein to R⁶ containing a —N═ ring atom in a ring refer to, for example, the ring nitrogen of a pyridine. As will be clear, reference to a —N═ atom indicates that the nitrogen has 3 bonds to it which are part of the ring structure (as in a pyridine nitrogen) and a —N═ atom does not refer to a —NH— or substituted —NH— ring member. Unless stated otherwise, R⁶ may be a mono-bicyclic or polycyclic heteroaryl ring system. It is to be understood that in addition to nitrogen R⁶ may also contain 1 or more additional heteroatoms (for example 1 to 4) selected from O, S and N and that these additional heteroatoms may be in the same ring or different rings to the —N═ atom.

It is to be understood that where it is stated that R⁶ contains a ring —N═ group and R⁶ contains at least one “unsubstituted —NH— group” in the heteroaryl ring(s), said —NH— is in addition to the —N═ group and is not substituted by an R³⁵ group. In addition to said —N═ and —NH— group R⁶ may contain one or more additional heteroatoms selected from O, S and N.

It is also to be understood that when R⁶ is a bicyclic or polycyclic heteroaryl which contains at least one unsubstituted —NH— ring member in addition to a —N═ ring atom, wherein the —NH— and ═N— group in R⁶ are attached to the same bridgehead ring atom at a junction of two fused rings in R⁶; then the —N═ and —NH— ring members are located in the ortho position to the same bridgehead atom (an atom at the junction of two fused rings) between two fused rings in R⁶, with the —N═ ring member in one of the fused rings and the —NH— ring member in the other fused ring. For example, R⁶ is a 9 to 11-membered fused bicyclic heteroaryl of the formula:

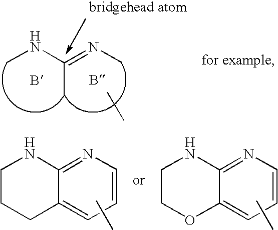

wherein B′ and B″ are, for example, both 5 or 6-membered monocyclic heteroaryl groups containing nitrogen and optionally one or more (for example 1 or 2) additional heteroatoms selected from O, S and N, or one of B′ and B is a 5 or 6-membered monocyclic heteroaryl group and the other is a 4 to 7 membered heterocyclic group, wherein B′ and B″ contain nitrogen and optionally one or more (for example 1 or 2) additional heteroatoms selected from O, S and N.

It is also to be understood that when R⁶ is substituted in an ortho position to the —N═ atom in R⁶ by at least one —NHR^{31a} group, the NHR^{31a} group is located on an adjacent ring atom to the —N═ group, for example R⁶ is:

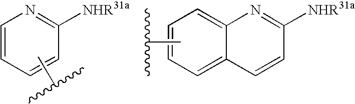

wherein [symbol] indicates the point of attachment of R⁶ to Z.

Similarly, when is stated that Z is NH and R⁶ is attached to Z by a ring carbon atom in an ortho position to the —N═ atom in R⁶, the carbon atom linked to Z is adjacent to the —

N=group in R⁶. For example, R⁶-Z- is a group of the formula:

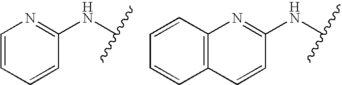

wherein [symbol] indicates the point of attachment of R⁶-Z- to —Y— in formula (I). In these embodiments when Z is stated to be NH, it is not substituted by R²⁶.

Unless stated otherwise R⁶ may optionally contain 1 or more additional heteroatoms selected from O, S and N, including one or more —NH— groups (which may be substituted by R³⁵) and R⁶ is optionally substituted on carbon by R³¹.

Where it is stated herein that the group Z-R⁶ has a pKa which is greater than or equal to about 6, the group Z-R⁶, together with any R³¹, R^{31a} or R³⁵ substituents has a pKa greater than or equal to about 6, for example a pKa in the range of from about 6 to about 12, for example from 6 to 9. The pKa of the group Z-R⁶ may be determined using routine methods. For example pKa may be measured using multiwavelength spectrophotometry to determine acid dissociation constants as described in:

Multiwavelength spectrophotometric determination of acid dissociation constants of ionizable drugs; Allen, R. I.; Box, K. J.; Corner, J. E. A.; Peake, C.; Tam, K. Y. Sirius Analytical Instruments Ltd, East Sussex, UK. Journal of Pharmaceutical and Biomedical Analysis (1998), 17(4,5); and
Multiwavelength spectrophotometric determination of acid dissociation constants Part IV. Water-insoluble pyridine derivatives. Tam, K. Y.; Hadley, M.; Patterson, W. Sirius Analytical Instruments Ltd., East Sussex, UK. Talanta (1999), 49(3), 539-546.

In particular the pKa may be determined using multiwavelength spectrophotometry in a Sirius GlpKa instrument equipped with the D-PAS accessory as follows. A stock solution of the compound in DMSO is prepared (1.5 mg/ml). 50 μl of this solution are added to 250 μl of phosphate buffer (2 mg/ml) and diluted in 20 ml of ionic strength adjusted water (KCl 0.15 M). The pH is then automatically adjusted to pH 2.5 with 0.5 M hydrochloric acid and the titration performed by adding 0.5 M potassium hydroxide. For each titration point the UV spectrum is recorded. The pKa values are calculated from the UV modifications with the Sirius pKaUV software.

There are also well known references that list the pKa values of various groups, for example:

Handbook of Biochemistry and Molecular Biology, Physical and Chemical Data, Vol. 1. 3rd Ed. Fasman, G. D.; Editor. (1976), 552 pp. Publisher: (CRC Press, Cleveland, Ohio).
Comprehensive Heterocyclic Chemistry II: An Extended and Updated Review of the Literature, 1982-1995, 2nd Edition. Katritzky, Alan R.; Rees, Charles W.; Scriven, Eric F. V; (1996), 9500 pp. Publisher: (Pergamon, Oxford, UK); and pKa Prediction for Organic Acids and Bases. Perrin, D. D.; Dempsey, Boyd; Serjeant, E. P. UK. (1981), 150 pp. Publisher: (Chapman and Hall, London).

Various software packages are also available for the estimation of pKa, for example ACD Labs.

It is to be understood that when n is 0, the thienyl group in formula (I) does not carry an R⁵ substituent.

The various functional groups and substituents making up the compounds of the formula (I) are typically chosen such that the molecular weight of the compound of the formula (I) does not exceed 1000. More usually, the molecular weight of the compound will be less than 750, for example less than 700, or less than 650, or less than 600, or less than 550. More preferably, the molecular weight is less than 525 and, for example, is 500 or less.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E- and Z- isomers). It is to be understood that the present invention encompasses all optical, diastereoisomers and geometric isomers and mixtures thereof that possess a5b1 inhibitory activity. The present invention also encompasses all tautomeric forms of the compounds of formula I that possess a5b1 antagonistic activity.

It is also to be understood that certain compounds of the formula I may exist in solvated as well as unsolvated forms such as, for example, hydrated forms. It is to be understood that the invention encompasses all such solvated forms that possess a5b1 antagonistic activity.

It is also to be understood that certain compounds of the formula I may exhibit polymorphism, and that the invention encompasses all such forms that possess a5b1 antagonistic activity.

Compounds of the formula (I) may exist in a number of different tautomeric forms and references to compounds of the formula (I) include all such forms. For the avoidance of doubt, where a compound can exist in one of several tautomeric forms and only one is specifically described or shown, all others are nevertheless embraced by formula (I). For example, when Z is NH and R⁶ is benzimidazolyl both tautomers of the benzimdazolyl group are possible for R⁶:

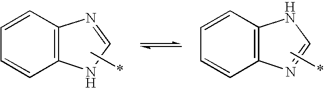

Other examples of tautomeric forms include keto-, enol-, and enolate-forms, as in, for example, the following tautomeric pairs: keto/enol, imine/enamine, amide/imino alcohol, amidine/amidine, nitroso/oxime, thioketone/enethiol, and nitro/aci-nitro.

Compounds of the formula (I) containing an amine function may also form N-oxides. A reference herein to a compound of the formula (I) that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the specification and claims includes both one and more than one such excipient.

A “pharmaceutically acceptable counter ion” means an ion having a charge opposite to that of the substance with which it is associated and that is pharmaceutically acceptable. Representative examples include, but are not limited to, chloride, bromide, iodide, methanesulfonate, p-tolylsulfonate, trifluoroacetate, acetate, and the like.

A “pharmaceutically acceptable salt” of a compound means a salt that is pharmaceutically acceptable and that possesses the desired pharmacological activity of the parent compound. Such pharmaceutically-acceptable salts of a compound of the formula I is, for example, an acid-addition salt of a compound of the formula I, for example an acid-addition salt with an inorganic or organic acid such as hydrochloric, hydrobromic, sulfuric, trifluoroacetic, citric or maleic acid; or, for example, a salt of a compound of the formula I which is sufficiently acidic, for example an alkali or alkaline earth metal salt such as a calcium or magnesium salt, or an ammonium salt, or a salt with an organic base such as methylamine, dimethylamine, trimethylamine, piperidine, morpholine or tris-(2-hydroxyethyl)amine.

In particular, compounds of the invention may form internal salts or zwitterions, and these form a particular aspect of the invention. Thus, for example, whilst the compounds are drawn and referred to in say the hydroxyl form, they may exist also in internal salt (zwitterionic) form, such as a zwitterion with a basic group in R⁶ as depicted below:

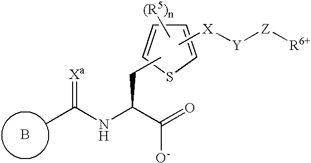

The representation of formula (I) and the examples of the present invention covers both hydroxyl and zwitterionic forms and mixtures thereof in all proportions.

“Leaving group” has the meaning conventionally associated with it in synthetic organic chemistry i.e., an atom or group capable of being displaced by a nucleophile and includes halogen (such as chloro, bromo, iodo), alkanesulfonyloxy (such as mesyloxy or trifluorosulfonyloxy) or arenesulfonyloxy (such as tosyloxy), and the like. Leaving Groups are well known in the art and are catalogued in “Protective Groups in Organic Synthesis 3^{rd} Ed.”, edited by Theodora Green and Peter Wuts (John Wiley, 1999).

The compounds of formula (I) may be administered in the form of a pro-drug which is broken down in the human or animal body to give a compound of the formula (I). A “Pro-drug” is any compound which releases an active parent drug according to formula (I) in vivo when such prodrug is administered to a mammalian subject. Prodrugs of a compound of formula (I) are prepared by modifying functional groups present in the compound of formula (I) in such a way that the modifications may be cleaved in vivo to release the parent compound. Examples of prodrugs include, but are not limited to esters (e.g., acetate, formate, and benzoate derivatives), carbamates (e.g., N,N-dimethylaminocarbonyl) of hydroxy or carboxy functional groups in compounds of formula (I), and the like.

Various forms of pro-drugs are known in the art. For examples of such pro-drug derivatives, see:
1. Design of Prodrugs, edited by H. Bundgaard, (Elsevier, 1985) and Methods in Enzymology, Vol. 42, p. 309-396, edited by K. Widder, et al. (Academic Press, 1985);
2. A Textbook of Drug Design and Development, edited by Krogsgaard-Larsen and H. Bundgaard, Chapter 5 “Design and Application of Prodrugs”, by H. Bundgaard p. 113-191 (1991);
3. H. Bundgaard, Advanced Drug Delivery Reviews, 8, 1-38 (1992);
4. H. Bundgaard, et al., Journal of Pharmaceutical Sciences, 77, 285 (1988);
5. N. Kakeya, et al., Chem Pharm Bull, 32, 692 (1984);
6. K. Beaumont et. al., Current Drug Metabolism, 4, 461 (2003).

An in-vivo hydrolysable ester of a compound of the Formula (I containing a carboxy or a hydroxy group is, for example, a pharmaceutically-acceptable ester which is hydrolysed in the human or animal body to produce the parent acid or alcohol. Suitable pharmaceutically-acceptable esters for carboxy include (1-6C)alkoxymethyl esters for example methoxymethyl, (1-6C)alkanoyloxymethyl esters for example pivaloyloxymethyl, phthalidyl esters, (3-8C)cycloalkoxycarbonyloxyC₁₋₆alkyl esters for example 1-cyclohexylcarbonyloxyethyl; 1,3-dioxolen-2-onylmethyl esters, for example 5-methyl-1,3-dioxolen-2-onylmethyl; and (1-6C)alkoxycarbonyloxyethyl esters.

An in-vivo hydrolysable ester of a compound of the formula (I) containing a hydroxy group includes inorganic esters such as phosphate esters (including phosphoramidic cyclic esters) and α-acyloxyalkyl ethers and related compounds which as a result of the in-vivo hydrolysis of the ester breakdown to give the parent hydroxy group/s. Examples of α-acyloxyalkyl ethers include acetoxymethoxy and 2,2-dimethylpropionyloxy-methoxy. A selection of in-vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl.

When R⁴ is other than H, the compounds of formula (I) may behave as pro-drugs with the R⁴ group being hydrolysed in-vivo to give the free carboxy group. As will be understood, such compounds and other prodrugs of formula (I) may exhibit low activity in the in-vitro assays described herein compared to the free carboxy compound. However, such compounds are expected to show activity under conditions that result in the hydrolysis of the R⁴ group to give the free carboxy group.

“Treating” or “treatment” of a disease includes:
1. preventing the disease, i.e. causing the clinical symptoms of the disease not to develop in a mammal that may be exposed to or predisposed to the disease but does not yet experience or display symptoms of the disease;
2. inhibiting the disease, i.e., arresting or reducing the development of the disease or its clinical symptoms; or
3. relieving the disease, i.e., causing regression of the disease or its clinical symptoms.
A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

The phrase “compound of the invention” means those compounds which are disclosed herein, both generically and specifically.

Particular novel compounds of the invention include, for example, compounds of the formulae, IA, IB, IB′, IC, IC′, ID and ID′ or pharmaceutically acceptable salts and pro-drugs thereof, wherein, unless otherwise stated, each of n, B, X^{a}, R⁴, R⁵, R⁶, R^{31a}, X, Y and Z has any of the meanings defined hereinbefore or in paragraphs (1) to (99) hereinafter:—

(1) B is a 5 or 6 membered monocyclic heteroaryl ring or a fully aromatic bicyclic 9 or 10 membered heteroaryl ring, which heteroaryl ring contains from 1 to 4 atoms selected from O, S and N, and wherein B is attached to the C(X^{a}) group by a ring carbon atom;

and wherein the heteroaryl ring B is substituted in an ortho position to the group C(X^{a}) by a R¹ substituent selected from halo, (1-3C)alkyl, cyclopropyl, halo-(1-3C)alkyl, (1-3C)alkoxy and (1-3C)alkylthio (particularly R¹ is selected from halo, (1-3C)alkyl, cyclopropyl and halo-(1-3C)alkyl);

and wherein the heteroaryl ring B optionally bears on carbon 1 or more (for example 1, 2 or 3) R³ substituents selected from halo, trifluoromethyl, cyano, isocyano, nitro, hydroxy, mercapto, amino, formyl, carboxy, carbamoyl, sulfamoyl, halo-(1-3C)alkyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

or from a group of the formula:

Q¹-X¹—

wherein X¹ is a direct bond or is selected from O, S, SO, SO₂, N(R⁷), C(O), CH(OR⁷), C(O)N(R⁷), N(R⁷)C(O), SO₂N(R⁷), N(R⁷)SO₂, OC(R⁷)₂, SC(R⁷)₂ and N(R⁷)C(R⁷)₂, wherein R⁷ is hydrogen or (1-6C)alkyl, and Q¹ is aryl, aryl-(1-6C)alkyl, (3-7C)cycloalkyl, (3-7C)cycloalkyl-(1-6C)alkyl, (3-7C)cycloalkenyl, (3-7C)cycloalkenyl-(1-6C)alkyl, heteroaryl, heteroaryl-(1-6C)alkyl, heterocyclyl or heterocyclyl-(1-6C)alkyl,

and wherein any carbon containing substituent on the heteroaryl ring B optionally bears on carbon one or more R⁸ groups,

and wherein if the heteroaryl ring B or any heteroaryl or heterocyclyl group which is a substituent on ring B contains an —NH— moiety, the nitrogen of said moiety optionally bears a group selected from R⁹,

and wherein any heterocyclyl group which is a substituent on ring B optionally bears 1 or 2 oxo or thioxo substituents,

or two adjacent substituents on the heteroaryl ring B optionally form a (1-3C)alkylenedioxy group;

R⁸ is selected from halo, trifluoromethyl, cyano, nitro, hydroxy, amino, carboxy, carbamoyl, sulfamoyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino, or from a group of the formula:

—X²—R¹⁰

wherein X² is a direct bond or is selected from O, C(O) and N(R¹¹), wherein R¹¹ is hydrogen or (1-6C)alkyl, and R¹⁰ is halo-(1-6C)alkyl, hydroxy-(1-6C)alkyl, (1-6C)alkoxy-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl and (1-6C)alkoxycarbonylamino-(1-6C)alkyl, or from a group of the formula:

—X³-Q²

wherein X³ is a direct bond or is selected from O, S, SO, SO₂, N(R¹²), C(O), CH(OR¹²), C(O)N(R¹²), N(R¹²)C(O), SO₂N(R¹²), N(R¹²)SO₂, OC(R¹²)₂, SC(R¹²)₂ and N(R¹²)C(R¹²)₂, wherein R¹² is hydrogen or (1-6C)alkyl, and Q² is aryl, aryl-(1-6C)alkyl, (3-7C)cycloalkyl, (3-7C)cycloalkyl-(1-6C)alkyl, (3-7C)cycloalkenyl, (3-7C)cycloalkenyl-(1-6C)alkyl, heteroaryl, heteroaryl-(1-6C)alkyl, heterocyclyl or heterocyclyl-(1-6C)alkyl,

and wherein R⁸ optionally bears on carbon one or more R¹³,

and wherein any if any heteroaryl or heterocyclyl group within R⁸ contains an —NH— moiety, the nitrogen of said moiety optionally bears a group selected from R¹⁴,

and wherein any heterocyclyl group within a substituent on R⁸ optionally bears 1 or 2 oxo or thioxo substituents;

R⁹ is selected from carbamoyl, sulfamoyl, (1-6C)alkyl, (2-8C)alkenyl, (2-8C)alkynyl, (1-6C)alkylsulfonyl, (1-6C)alkoxycarbonyl, N-(1-6C)alkylcarbamoyl, N,N-di-[(1-6C)alkyl]carbamoyl, (2-6C)alkanoyl, N-(1-6C)alkylsulfamoyl and N,N-di-[(1-6C)alkyl]sulfamoyl,

or from a group of the formula:

—X⁴—R¹⁵

wherein X⁴ is a direct bond or is selected from C(O), SO₂, C(O)N(R¹⁶) and SO₂N(R¹⁶), wherein R¹⁶ is hydrogen or (1-6C)alkyl, and R¹⁵ is halo-(1-6C)alkyl, hydroxy-(1-6C)alkyl, (1-6C)alkoxy-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl and (1-6C)alkoxycarbonylamino-(1-6C)alkyl,

or from a group of the formula:

—X⁵-Q³

wherein X⁵ is a direct bond or is selected from C(O), SO₂, C(O)N(R¹⁷) and SO₂N(R¹⁷), wherein R¹⁷ is hydrogen or (1-6C)alkyl, and Q³ is aryl, aryl-(1-6C)alkyl, (3-7C)cycloalkyl, (3-7C)cycloalkyl-(1-6C)alkyl, (3-7C)cycloalkenyl, (3-7C)cycloalkenyl-(1-6C)alkyl, heteroaryl, heteroaryl-(1-6C)alkyl, heterocyclyl or heterocyclyl-(1-6C)alkyl,

and wherein R⁹ optionally bears on carbon one or more R¹⁸,

and wherein any if any heteroaryl or heterocyclyl group within R⁹ contains an —NH— moiety, the nitrogen of said moiety optionally bears a group selected from R¹⁹,

and wherein any heterocyclyl group within a substituent on R⁹ optionally bears 1 or 2 oxo or thioxo substituents;

R¹³ and R¹⁸ are each independently selected from halo, cyano, hydroxy, carboxy, amino, (3-6C)cycloalkyl, (2-6C)alkenyl, (2-6C)alkynyl, (1-6C)alkoxy, (1-6C)alkylamino and di-[(1-6C)alkyl]amino;

R¹⁴ and R¹⁹ are each independently selected from carbamoyl, sulfamoyl, (1-6C)alkyl, (2-8C)alkenyl, (2-8C)alkynyl, (1-6C)alkylsulfonyl, N-(1-6C)alkylcarbamoyl, N,N-di-[(1-6C)alkyl]carbamoyl, (2-6C)alkanoyl, N-(1-6C)alkylsulfamoyl and N,N-di-[(1-6C)alkyl]sulfamoyl,

or from a group of the formula:

—X⁶-Q⁴

wherein X⁶ is a direct bond or is selected from C(O), SO₂, C(O)N(R²⁰) and SO₂N(R²⁰), wherein R²⁰ is hydrogen or (1-6C)alkyl, and Q⁴ is (3-7C)cycloalkyl or (3-7C)cycloalkyl-(1-6C)alkyl.
(2) B is a 5 or 6 membered monocyclic heteroaryl ring or a fully aromatic bicyclic 9 or 10 membered heteroaryl ring, which heteroaryl ring contains from 1 to 4 atoms selected from O, S and N, and wherein B is attached to the C(X^{a}) group by a ring carbon atom;

and wherein the heteroaryl ring B is substituted in an ortho position to the group C(X^{a}) by a R¹ substituent selected from fluoro, chloro, bromo, (1-3C)alkyl, cyclopropyl and trifluoromethyl (for example R¹ is selected from chloro, bromo, (1-3C)alkyl, cyclopropyl and trifluoromethyl);

and wherein the heteroaryl ring B optionally bears on carbon 1 or more (for example 1, 2 or 3) R³ substituents selected from halo, trifluoromethyl, cyano, nitro, hydroxy, mercapto, amino, formyl, carboxy, carbamoyl, sulfamoyl, (1-4C)alkyl, (2-4C)alkenyl, (2-4C)alkynyl, (1-4C)alkoxy, (2-4C)alkenyloxy, (2-4C)alkynyloxy, (1-4C)alkylthio, (1-4C)alkylsulfinyl, (1-4C)alkylsulfonyl, (1-4C)alkylamino, di-[(1-4C)alkyl]amino, (1-4C)alkoxycarbonyl, N-(1-4C)alkylcarbamoyl, N,N-di-[(1-4C)alkyl]carbamoyl, (2-4C)alkanoyl, (2-4C)alkanoyloxy, (2-4C)alkanoylamino, N-(1-4C)alkyl-(2-4C)alkanoylamino, N-(1-4C)alkylsulfamoyl, N, N-di-[(1-4C)alkyl]sulfamoyl, (1-4C)alkanesulfonylamino and N-(1-4C)alkyl-(1-4C)alkanesulfonylamino,

and wherein any carbon containing substituent on the heteroaryl ring B optionally bears on carbon one or more substituents selected from halo, cyano, hydroxy, carboxy, amino, (3-6C)cycloalkyl, (1-4C)alkoxy, (1-4C)alkylamino and di-[(1-4C)alkyl]amino,

and wherein if the heteroaryl ring B contains an —NH— moiety, the nitrogen of said moiety optionally bears a group selected from carbamoyl, sulfamoyl, (1-4C)alkyl, (2-4C)alkenyl, (2-4C)alkynyl, (1-4C)alkylsulfonyl, N-(1-4C)alkylcarbamoyl, N,N-di-[(1-4C)alkyl]carbamoyl, (2-4C)alkanoyl, N-(1-4C)alkylsulfamoyl and N,N-di-[(1-4C)alkyl]sulfamoyl,

or two adjacent substituents on ring B optionally form a (1-3C)alkylenedioxy group,

and wherein ring B is linked to the C(X^{a}) group by a carbon atom.

(3) B is a 5 or 6 membered monocyclic heteroaryl ring or a fully aromatic bicyclic 9 or 10 membered heteroaryl ring, which heteroaryl ring contains from 1 to 4 atoms selected from O, S and N, and wherein B is attached to the C(X^{a}) group by a ring carbon atom;

and wherein the heteroaryl ring B is substituted in an ortho position to the group C(X^{a}) by a R¹ substituent selected from fluoro, chloro, bromo, (1-3C)alkyl, cyclopropyl and trifluoromethyl (for example R¹ is selected from chloro, bromo, (1-3C)alkyl, cyclopropyl and trifluoromethyl);

and wherein the heteroaryl ring B optionally bears on carbon 1 or more (for example 1, 2 or 3) R³ substituents selected from halo, trifluoromethyl (1-4C)alkyl, (2-4C)alkenyl, (2-4C)alkynyl and (1-4C)alkoxy,

or two adjacent substituents on ring B optionally form a (1-3C)alkylenedioxy group,

and wherein if the heteroaryl ring B contains an —NH— moiety, the nitrogen of said moiety optionally bears a substituent selected from (1-4C)alkyl, (2-4C)alkenyl and (2-4C)alkynyl;

and wherein any carbon containing substituent on the heteroaryl ring B optionally bears on carbon one or more substituents selected from hydroxy and (1-4C)alkoxy.

(4) B is a 5 or 6 membered monocyclic heteroaryl ring, which heteroaryl ring contains from 1 to 4 atoms selected from O, S and N, and wherein B is attached to the C(X^{a}) group by a ring carbon atom;

and wherein the heteroaryl ring B is substituted in an ortho position to the group C(X^{a}) by a R¹ substituent selected from fluoro, chloro, bromo, (1-3C)alkyl, cyclopropyl and trifluoromethyl (for example R¹ is selected from chloro, bromo, (1-3C)alkyl, cyclopropyl and trifluoromethyl);

and wherein the heteroaryl ring B optionally bears on carbon 1 or more (for example 1, 2 or 3) R³ substituents selected from halo, trifluoromethyl, (1-4C)alkyl, (2-4C)alkenyl, (2-4C)alkynyl and (1-4C)alkoxy,

or two adjacent substituents on ring B optionally form a (1-3C)alkylenedioxy group,

and wherein if the heteroaryl ring B contains an —NH— moiety, the nitrogen of said moiety optionally bears a group selected from (1-4C)alkyl, (2-4C)alkenyl and (2-4C)alkynyl;

and wherein any carbon containing substituent on the heteroaryl ring B optionally bears on carbon one or more substituents selected from hydroxy and (1-4C)alkoxy.

(5) B is a 5 or 6 membered monocyclic heteroaryl ring or a fully aromatic bicyclic 9 or 10 membered heteroaryl ring, which heteroaryl ring contains from 1 to 4 atoms selected from O, S and N, and wherein B is attached to the C(X^{a}) group by a ring carbon atom;

and wherein the heteroaryl ring B is substituted in an ortho position to the group C(X^{a}) by a R¹ substituent which is halo (particularly chloro or bromo, more particularly chloro);

and wherein the heteroaryl ring B optionally bears on carbon 1 or more (for example 1, 2 or 3) R³ substituents selected from halo, trifluoromethyl (1-4C)alkyl, (2-4C)alkenyl, (2-4C)alkynyl and (1-4C)alkoxy,

and wherein if the heteroaryl ring B contains an —NH— moiety, the nitrogen of said moiety optionally bears a group selected from (1-4C)alkyl, (2-4C)alkenyl and (2-4C)alkynyl;

and wherein any carbon containing substituent on the heteroaryl ring B optionally bears on carbon one or more substituents selected from hydroxy and (1-4C)alkoxy,

or two adjacent substituents on ring B optionally form a (1-3C)alkylenedioxy group.

(6) B—C(X^{a}) is a group of sub-formula (a) or (b) above and R¹ is selected from chloro, (1-3C)alkyl, cyclopropyl and trifluoromethyl; and wherein X^{a}, X^{b}, X^{c}, X^{d}, X^{e}, X^{f}, R², R^{3a}, R^{3b} and R^{3c} are as hereinbefore defined.
(7) B—C(X^{a}) is a group of sub-formula (a) or (b) above and R¹ is selected from chloro and (1-3C)alkyl; and wherein X^{a}, X^{b}, X^{c}, X^{d}, X^{e}, R², R^{3a}, R^{3b} and R^{3c} are as hereinbefore defined.
(8) B—C(X^{a}) is a group of sub-formula (a) or (b) above and R¹ is selected from chloro, methyl and ethyl; and wherein X^{a}, X^{b}, X^{c}, X^{d}, X^{e}, X^{f}, R², R^{3a}, R^{3b} and R^{3c} are as hereinbefore defined.
(9) B—C(X^{a}) is a group of sub-formula (a) or (b) above and R¹ is methyl or ethyl and wherein X^{a}, X^{b}, X^{c}, X^{d}, R^{e}, X^{f}, R², R^{3a}, R^{3b} and R^{3c} are as hereinbefore defined.
(10) B—C(X^{a}) is a group of sub-formula (a) or (b) above and R¹ is methyl or trifluoromethyl and wherein X^{a}, X^{b}, X^{c}, X^{d}, X^{e}, X^{f}, R², R^{3a}, R^{3b} and R^{3c} are as hereinbefore defined.
(11) B—C(X^{a}) is a group of sub-formula (a) or (b) above and R¹ is chloro or methyl; and wherein X^{a}, X^{b}, X^{c}, X^{d}, X^{e}, R², R^{3a}, R^{3b} and R^{3c} are as hereinbefore defined.
(12) B—C(X^{a}) is a group of sub-formula (a) or (b) above and R¹ is chloro; and wherein X^{a}, X^{b}, X^{c}, X^{d}, X^{e}, X^{f}, R², R^{3a}, R^{3b} and R^{3c} are as hereinbefore defined.
(13) B—C(X^{a}) is a group of sub-formula (a) or (b) above and R¹ is methyl; and wherein X^{a}, X^{b}, X^{c}, X^{d}, X^{e}, X^{f}, R², R^{3b} and R^{3c} are as hereinbefore defined.
(14) B—C(X^{a}) is a group of sub-formula (a) or (b) above and R² is selected from hydrogen, halo, trifluoromethyl, (1-6C)alkyl, and (1-6C)alkoxy; and wherein X^{a}, X^{b}, X^{c}, X^{d}, X^{e}, X^{f}, R¹, R^{3a}, R^{3b} and R^{3c} are as hereinbefore defined.
(15) B—C(X^{a}) is a group of sub-formula (a) or (b) above and R² is selected from hydrogen, halo and (1-4C)alkyl, or R² and an adjacent R³ group optionally form a (1-3C)alkylenedioxy group (for example methylenedioxy or ethylenedioxy); and wherein X^{a}, X^{b}, X^{c}, X^{d}, X^{e}, X^{f}, R¹, R^{3a}, R^{3b} and R^{3c} are as hereinbefore defined.
(16) B—C(X^{a}) is a group of sub-formula (a) or (b) above and R² is selected from hydrogen, fluoro, chloro, bromo and methyl; and wherein X^{a}, X^{b}, X^{c}, X^{d}, X^{e}, X^{f}, R¹, R^{3a}, R^{3b} and R^{3c} are as hereinbefore defined.
(17) B—C(X^{a}) is a group of sub-formula (a) or (b) above and R² is selected from hydrogen and chloro; and wherein X^{a}, X^{b}, X^{c}, X^{d}, X^{e}, X^{f}, R¹, R^{3a}, R^{3b} and R^{3c} are as hereinbefore defined.
(18) B—C(X^{a}) is a group of sub-formula (a) or (b) above and R² is hydrogen; and wherein X^{a}, X^{b}, X^{c}, X^{d}, X^{e}, X^{f}, R¹, R^{3a}, R^{3b} and R^{3c} are as hereinbefore defined.
(19) B—C(X^{a}) is a group of sub-formula (a) or (b) above and R² is fluoro or chloro; and wherein X^{a}, X^{b}, X^{c}, X^{d}, X^{e}, X^{f}, R¹, R^{3a}, R^{3b} and R^{3c} are as hereinbefore defined.
(20) B—C(X^{a}) is a group of sub-formula (a) or (b) above and R² is chloro; and wherein X^{a}, X^{b}, X^{c}, X^{d}, X^{e}, X^{f}, R¹, R^{3a}, R^{3b} and R^{3c} are as hereinbefore defined.
(21) B—C(X^{a}) is a group of sub-formula (a) or (b) above and R¹ is chloro or methyl and R² is selected from hydrogen, fluoro, chloro and methyl; and wherein X^{a}, X^{b}, X^{c}, X^{d}, X^{e}, X^{f}, R^{3a}, R^{3b} and R^{3c} are as hereinbefore defined.
(22) B—C(X^{a}) is a group of sub-formula (a) or (b) above and R¹ and R² are both chloro; and wherein X^{a}, X^{b}, X^{c}, X^{d}, X^{e}, X^{f}, R^{3a}, R^{3b} and R^{3c} are as hereinbefore defined.
(23) B—C(X^{a}) is a group of sub-formula (a) or (b) above and R^{3a}, R^{3b} and R^{3c}, which may be the same or different, are selected from halo, trifluoromethyl, cyano, nitro, hydroxy, mercapto, amino, carboxy, carbamoyl, sulfamoyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino, or from a group of the formula:

Q¹-X¹—

wherein X¹ is a direct bond or is selected from O, S, SO, SO₂, N(R⁷), C(O), CH(OR⁷), C(O)N(R⁷), N(R⁷)C(O), SO₂N(R⁷), N(R⁷)SO₂, OC(R⁷)₂, SC(R⁷)₂ and N(R⁷)C(R⁷)₂, wherein R⁷ is hydrogen or (1-6C)alkyl, and Q¹ is (3-7C)cycloalkyl or (3-7C)cycloalkyl-(1-6C)alkyl,

and wherein R^{3a}, R^{3b} and R^{3c} optionally bears on carbon one or more substituents selected from halo, trifluoromethyl, cyano, nitro, hydroxy, amino, carboxy, carbamoyl, sulfamoyl, (1-6C)alkyl, (2-8C)alkenyl, (2-8C)alkynyl, (1-6C)alkoxy, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

or two or R^{3a}, R^{3b} and R^{3c} substituents optionally form a (1-3C)alkylenedioxy group;

and wherein R¹ and R² are has hereinbefore defined, for example as defined in any one of paragraphs (6) to (22) above, particularly R¹ and R² are as defined in paragraph (21) or (22) above,

and wherein X^{a}, X^{b}, X^{c}, X^{d}, X^{e} and X^{f} are as hereinbefore defined.

(24) B—C(X^{a}) is a group of sub-formula (a) or (b) above and R^{3a}, R^{3b} and R^{3c}, which may be the same or different, is selected from halo, trifluoromethyl, hydroxy, amino, (1-6C)alkyl, (2-8C)alkenyl, (2-8C)alkynyl, (1-6C)alkoxy, (1-6C)alkylsulfonyl, (1-6C)alkylamino, di-[(1-6C)alkyl]amino, (1-6C)alkoxycarbonyl, (2-6C)alkanoyl, (2-6C)alkanoyloxy, amino-(1-6C)alkyl, (1-6C)alkylamino-(1-6C)alkyl and di-[(1-6C)alkyl]amino-(1-6C)alkyl,

or an adjacent two of R^{3a}, R^{3b} and R^{3c} substituents optionally form a (1-3C)alkylenedioxy group;

and wherein R¹ and R² are has hereinbefore defined, for example as defined in any one of paragraphs (6) to (22) above, particularly R¹ and R² are as defined in paragraph (21) or (22) above,

and wherein X^{a}, X^{b}, X^{c}, X^{d}, X^{e} and X^{f} are as hereinbefore defined.

(25) B—C(X^{a}) is a group of sub-formula (a) or (b) above and R^{3a}, R^{3b} and R^{3c}, which may be the same or different, are selected from halo and (1-4C)alkyl, or two adjacent R^{3a}, R^{3b} and R^{3c} substituents optionally form a (1-3C)alkylenedioxy group;

and wherein R¹ and R² are has hereinbefore defined, for example as defined in any one of paragraphs (6) to (22) above, particularly R¹ and R² are as defined in paragraph (21) or (22) above,

and wherein X^{a}, X^{b}, X^{c}, X^{d}, X^{e} and X^{f} are as hereinbefore defined.

(26) B—(X^{a}) is a group of sub-formula (a) or (b) above and R^{3a}, R^{3b} and R^{3c}, which may be the same or different, is selected from fluoro, chloro, bromo and (1-3C)alkyl;

and wherein R¹ and R² are has hereinbefore defined, for example as defined in any one of paragraphs (6) to (22) above, particularly R¹ and R² are as defined in paragraph (21) or (22) above,

and wherein X^{a}, X^{b}, X^{c}, X^{d}, X^{e} and X^{f} are as hereinbefore defined.

(27) B—(X³) is a group of sub-formula (a) or (b) above and one of R^{3a}, R^{3b} and R^{3c} is selected from fluoro, chloro and methyl, and the others are hydrogen;

and wherein R¹ and R² are has hereinbefore defined, for example as defined in any one of paragraphs (6) to (22) above, particularly R¹ and R² are as defined in paragraph (21) or (22) above,

and wherein X^{a}, X^{b}, X^{c}, X^{d}, X^{e} and X^{f} are as hereinbefore defined.

(28) B—(X^{a}) is a group of sub-formula (a) or (b) above and R^{3a}, R^{3b} and R^{3c} are all hydrogen;

and wherein R¹ and R² are has hereinbefore defined, for example as defined in any one of paragraphs (6) to (22) above, particularly R¹ and R² are as defined in paragraph (21) or (22) above,

and wherein X^{a}, X^{b}, X^{c}, X^{d}, X^{e} and X^{f} are as hereinbefore defined.

(29) B is selected from isoxazolyl, 1,3-oxazolyl, thienyl, furyl, 1,3-thiazolyl, pyrrolyl, pyrazolyl, pyridyl, benzofuranyl and indolyl, wherein B is attached to the C(X^{a}) group by a ring carbon atom;

and wherein B is substituted in an ortho position to the group C(X^{a}) by a R¹ substituent as hereinbefore defined, for example R¹ is selected from halo, (1-3C)alkyl, (1-3C)alkyl, (1-3C)alkoxy and (1-3C)alkylthio (particularly a substituent selected from chloro, methyl, ethyl, methoxy, methylthio and ethylthio, more particularly a substituent selected from chloro, methyl and ethyl);

and wherein B optionally bears on carbon 1 or 2 substituents selected from halo, trifluoromethyl (1-3C)alkyl and (1-4C)alkoxy;

and wherein if B contains an —NH— moiety, the nitrogen of said moiety optionally bears a (1-3C)alkyl group.

(30) B is selected from 2-furyl, 3-furyl, 2-pyrrolyl, 2-thienyl, 3-thienyl, 4-oxazolyl, 5-oxazolyl, 4-isoxazolyl, 4-pyrazolyl, 5-pyrazolyl, 5-thiazolyl, 2-pyridyl, 3-pyridyl, 4-pyridyl or 2-benzofuryl (particularly B is 4-pyridyl or isoxazol-4-yl, more particularly B is isoxazol-4-yl);

wherein B is substituted in an ortho position to the group C(X^{a}) by a R¹ substituent and optionally bears 1 or 2 R³ wherein R¹ and R³ are as defined in any one of (1) to (5) above.

(31) B is as defined is any one of (1) to (30), wherein B carries at least 1 halo substituent. Suitably in this embodiment B is substituted in an ortho position to the group C(X^{a}) by chloro or bromo, particularly chloro.
(32) B is selected from 3-chloro-2-thienyl, 3-methyl-2-thienyl, 2-methyl-3-thienyl, 2-ethyl-3-thienyl, 2-methoxy-3-thienyl, 4-methyl-3-thienyl, 4-ethyl-3-thienyl, 4-methoxy-3-thienyl, 3-chloro-4-methyl-2-thienyl, 2,5-dichloro-3-thienyl, 1-methyl-1H-pyrrol-2-yl, 3-methyl-1H-pyrazol-4-yl, 3,5-dimethyl-1H-pyrazol-4-yl, 1,3,5-trimethyl-1H-pyrazol-4-yl, 1-ethyl-3-methyl-1H-pyrazol-5-yl, 3-methyl-2-furyl, 2-methyl-3-furyl, 4-methyl-1,3-thiazol-5-yl, 2,4-dimethyl-1,3-thiazol-5-yl, 3-methylisoxazol-4-yl, 3,5-dimethylisoxazol-4-yl, 2,5-dimethyl-1,3-oxazol-4-yl, 4-methyl-1,3-oxazol-5-yl, 3-chloropyridin-2-yl, 3-methylpyridin-2-yl, 3,6-dichloropyridin-2-yl, 3,5-dichloropyridin-2-yl, 3-chloro-5-(trifluoromethyl)pyridin-2-yl, 2-chloro-6-methylpyridin-3-yl, 4-methylpyridin-3-yl, 4-chloropyridin-3-yl, 2-methylpyridin-3-yl, 2-chloropyridin-3-yl, 3-chloropyridin-4-yl, 3-bromopyridin-4-yl, 3-methoxypyridin-4-yl, 3-methylpyridin-4-yl, 3-ethylpyridin-4-yl, 3-(methylthio)pyridin-4-yl, 3-(ethylthio)pyridin-4-yl, 3,5-dichloropyridin-4-yl, 3,5-dibromopyridin-4-yl, 3,5-dimethylpyridin-4-yl, 3,5-diethylpyridin-4-yl, 2-(ethylthio)pyridin-3-yl, 2,6-dichloropyridin-3-yl, 2,6-dimethoxypyridin-3-yl, 3-chlorol-methyl-1H-indol-2-yl and 3-methyl-1-benzofuran-2-yl. Particularly B is selected from 3-chloro-2-thienyl, 3-methyl-2-thienyl, 3-chloro-4-methyl-2-thienyl, 2,5-dichloro-3-thienyl, 1-methyl-1H-pyrrol-2-yl, 3-methyl-1H-pyrazol-4-yl, 3,5-dimethyl-1H-pyrazol-4-yl, 1,3,5-trimethyl-1H-pyrazol-4-yl, 1-ethyl-3-methyl-1H-pyrazol-5-yl, 3-methyl-2-furyl, 2-methyl-3-furyl, 4-methyl-1,3-thiazol-5-yl, 2,4-dimethyl-1,3-thiazol-5-yl, 3-methylisoxazol-4-yl, 3,5-dimethylisoxazol-4-yl, 2,5-dimethyl-1,3-oxazol-4-yl, 4-methyl-1,3-oxazol-5-yl, 3-chloropyridin-2-yl, 3-methylpyridin-2-yl, 3,6-dichloropyridin-2-yl, 3,5-dichloropyridin-2-yl, 3-chloro-5-(trifluoromethyl)pyridin-2-yl, 2-chloro-6-methylpyridin-3-yl, 4-methylpyridin-3-yl, 2-methylpyridin-3-yl, 2-chloropyridin-3-yl, 3-methylpyridin-4-yl, 3,5-dichloropyridin-4-yl, 2-(ethylthio)pyridin-3-yl, 2,6-dichloropyridin-3-yl, 2,6-dimethoxypyridin-3-yl and 3-methyl-1-benzofuran-2-yl.
(33) B is selected from 3-chloro-2-thienyl, 3,5-dimethylisoxazol-4-yl and 3,5-dichloropyridin-4-yl (particularly B is 3,5-dimethylisoxazol-4-yl or 3,5-dichloropyridin-4-yl, for example B is 3,5-dimethylisoxazol-4-yl).
(34) R⁴ is selected from hydrogen and (1-6C)alkyl, wherein R⁴ optionally bears on carbon one or more R²¹ substituents selected from halo, hydroxy and (1-4C)alkoxy;
(35) R⁴ is selected from hydrogen and (1-4C)alkyl, wherein R⁴ optionally bears on carbon a hydroxy substituent, for example R⁴ is selected from hydrogen, methyl, ethyl, propyl, isopropyl, butyl, isobutyl, tert-butyl 2-hydroxyethyl and 3-hydroxybutyl;
(36) R⁴ is selected from monocyclic heterocyclyl or heterocyclyl(1-6C)alkyl, and wherein and wherein any heterocyclyl group within R⁴ optionally bears 1 or 2 oxo substituent.
(37) R⁴ is selected from hydrogen, methyl, ethyl, hydroxyethyl, iso-propyl, 2-(diethylaminoethyl),

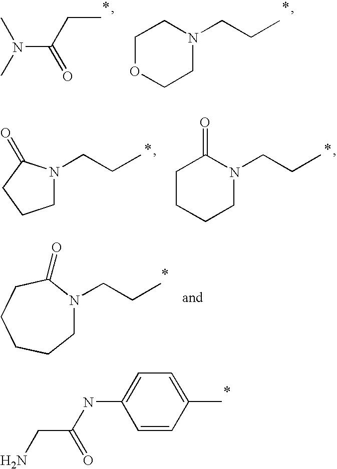

where * indicates the point of attachment to the oxygen atom,
(38) R⁴ is hydrogen;
(39) n is 0, 1 or 2 and each R⁵, which may be the same or different, is selected from halo, trifluoromethyl, cyano, nitro, hydroxy, amino, carboxy, carbamoyl, sulfamoyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino, or from a group of the formula:

Q⁵-X⁷—

wherein X¹ is a direct bond or is selected from O, S, SO, SO₂, N(R²³), C(O), CH(OR²³), C(O)N(R²³), N(R²³)C(O), SO₂N(R²³), N(R²³)SO₂, OC(R²³)₂, SC(R²³)₂ and N(R²³)C(R²³)₂, wherein R²³ is hydrogen or (1-6C)alkyl, and Q⁵ is phenyl-(1-6C)alkyl, (3-6C)cycloalkyl, (3-6C)cycloalkyl-(1-6C)alkyl, heteroaryl-(1-6C)alkyl, heterocyclyl or heterocyclyl-(1-6C)alkyl,

and wherein R⁵ optionally bears on carbon one or more R²⁴ substituents selected from halo, cyano, hydroxy, carboxy, amino, (3-6C)cycloalkyl, (2-6C)alkenyl, (2-6C)alkynyl, (1-6C)alkoxy, (1-6C)alkylamino and di-[(1-6C)alkyl]amino,

and wherein any if any heteroaryl or heterocyclyl group within R⁵ contains an —NH— moiety, the nitrogen of said moiety optionally bears an R²⁵ selected from carbamoyl, sulfamoyl, (1-6C)alkyl, (2-8C)alkenyl, (2-8C)alkynyl, (1-6C)alkylsulfonyl, N-(1-6C)alkylcarbamoyl, N,N-di-[(1-6C)alkyl]carbamoyl, (2-6C)alkanoyl, N-(1-6C)alkylsulfamoyl and N,N-di-[(1-6C)alkyl]sulfamoyl, or from a group of the formula:

—X⁶-Q⁴

wherein X⁶ is a direct bond or is selected from C(O), SO₂, C(O)N(R²⁰) and SO₂N(R²⁰), wherein R²⁰ is hydrogen or (1-6C)alkyl, and Q⁴ is (3-7C)cycloalkyl or (3-7C)cycloalkyl-(1-6C)alkyl;

and wherein any heterocyclyl group within R⁵ optionally bears 1 oxo substituent;

or two R⁵ substituents optionally form a (1-3C)alkylenedioxy group,

with the proviso that when X⁷ is a direct bond then Q⁵ is not phenyl or heteroaryl.

(40) n is 0, 1 or 2 and each R⁵, which may be the same or different, is selected from halo, trifluoromethyl, cyano, hydroxy, amino, carboxy, (1-6C)alkyl, (2-8C)alkenyl, (2-8C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, (2-6C)alkanoyl and (2-6C)alkanoyloxy, or from a group of the formula:

Q⁵-X⁷—

wherein X⁷ is a direct bond or is selected from O, S, SO, SO₂, N(R²³) and C(O), wherein R²³ is hydrogen or (1-6C)alkyl, and Q⁵ is (3-6C)cycloalkyl, (3-6C)cycloalkyl-(1-6C)alkyl, heterocyclyl or heterocyclyl-(1-6C)alkyl, which heterocyclyl is a saturated monocyclic 4 to 7 membered heterocyclyl group containing 1 or 2 heteroatoms selected from O, S and N,

and wherein R⁵ optionally bears on carbon one or more R²⁴ substituents selected from halo, cyano, hydroxy, carboxy, amino, (3-6C)cycloalkyl, (2-6C)alkenyl, (2-6C)alkynyl, (1-6C)alkoxy, (1-6C)alkylamino and di-[(1-6C)alkyl]amino,

and wherein any if any heterocyclyl group within R⁵ contains an —NH— moiety, the nitrogen of said moiety optionally bears an R²⁵ selected from carbamoyl, sulfamoyl, (1-6C)alkyl, (2-8C)alkenyl, (2-8C)alkynyl, (1-6C)alkylsulfonyl, N-(1-6C)alkylcarbamoyl, N,N-di-[(1-6C)alkyl]carbamoyl, (2-6C)alkanoyl, N-(1-6C)alkylsulfamoyl and N,N-di-[(1-6C)alkyl]sulfamoyl, or from a group of the formula:

—X⁶-Q⁴

wherein X⁶ is a direct bond or is selected from C(O), SO₂, C(O)N(R²⁰) and SO₂N(R²⁰), wherein R²⁰ is hydrogen or (1-6C)alkyl, and Q⁴ is (3-7C)cycloalkyl or (3-7C)cycloalkyl-(1-6C)alkyl;

and wherein any heterocyclyl group within R⁵ optionally bears 1 oxo substituent.

(41) n is 0, 1 or 2;

each R⁵, which may be the same or different, is selected from halo, trifluoromethyl, cyano, nitro, mercapto, amino, formyl, carboxy, carbamoyl, sulfamoyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

and wherein R⁵ optionally bears on carbon one or more R²⁴ groups selected from halo, cyano, hydroxy, carboxy, amino, (3-6C)cycloalkyl, (2-6C)alkenyl, (2-6C)alkynyl, (1-6C)alkoxy, (1-6C)alkylamino and di-[(1-6C)alkyl]amino,

or two R⁵ substituents optionally form a (1-3C)alkylenedioxy group.

(42) n is 0, 1 or 2 and each R⁵, which may be the same or different, is selected from halo, trifluoromethyl, cyano, hydroxy, amino, (1-4C)alkyl, (2-4C)alkenyl, (2-4C)alkynyl, (1-4C)alkoxy(1-4C)alkylamino and di-[(1-4C)alkyl]amino, or from a group of the formula:

Q⁵-X⁷—

wherein X⁷ is a direct bond or is selected from O and N(R²³) wherein R²³ is hydrogen or (1-4C)alkyl, and Q⁵ is (3-6C)cycloalkyl, (3-6C)cycloalkyl-(1-4C)alkyl, heterocyclyl or heterocyclyl-(1-4C)alkyl, which heterocyclyl is a saturated monocyclic heterocyclyl group containing 1 or 2 heteroatoms selected from O, S and N,

and wherein R⁵ optionally bears on carbon one or more R²⁴ substituents selected from halo, cyano, hydroxy, amino, (1-4C)alkoxy, (1-4C)alkylamino and di-[(1-4C)alkyl]amino,

and wherein any if any heterocyclyl group within R⁵ contains an —NH— moiety, the nitrogen of said moiety optionally bears an R²⁵ selected from (1-4C)alkyl, (1-4C)alkylsulfonyl and (2-4C)alkanoyl or from a group of the formula:

—X⁶-Q⁴

wherein X⁶ is a direct bond or is selected from C(O) and SO₂, and Q⁴ is (3-7C)cycloalkyl or (3-7C)cycloalkyl-(1-6C)alkyl;

and wherein any heterocyclyl group within R⁵ optionally bears 1 oxo substituent.

(43) n is 0, 1 or 2 and each R⁵, which may be the same or different, is selected from halo, hydroxy, amino, (1-4C)alkyl, (1-4C)alkoxy(1-4C)alkylamino and di-[(1-4C)alkyl]amino,

and wherein R⁵ optionally bears on carbon one or more R²⁴ substituents selected from halo, hydroxy, amino, (1-4C)alkoxy, (1-4C)alkylamino and di-[(1-4C)alkyl]amino.

(44) n is 0.
(45) n is 1 and R⁵ is as hereinbefore defined, for example as defined in any of (39) to (43) above.

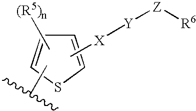

(46) The group: in formula I is:

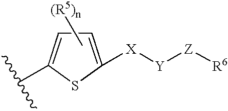

wherein n, R⁵, R⁶, X, Y and Z are as hereinbefore defined.
(47) X and Z, which may be the same or different, are selected from a direct bond, N(R²⁶), O, S, (1-6C)alkylene, CH═CH and C≡C, wherein R²⁶ is hydrogen, (1-6C)alkyl or (3-7C)cycloalkyl, and wherein any X and Z optionally bears on carbon one or more R²⁸ substituents wherein R²⁸ is as hereinbefore defined.
(48) X is selected from a direct bond, NR²⁶, O and (1-6C)alkylene, wherein R²⁶ is hydrogen, (1-6C)alkyl or (3-7C)cycloalkyl, and wherein any X optionally bears on carbon one or more R²⁸ substituents wherein R²⁸ is as hereinbefore defined.
(49) X is selected from a direct bond and O.

### (50) X is O.

(51) X is a direct bond.
(52) —Y is selected from (1-6C)alkylene and (3-7C)cycloalkylene,

and wherein adjacent carbon atoms in any (2-6C)alkylene chain within a Y substituent are optionally separated by the insertion into the chain of a group selected from O, N(R²⁷), N(R²⁷)C(O), C(O)N(R²⁷), CH═CH and C≡C wherein R²⁷ is hydrogen, (1-6C)alkyl or (3-7C)cycloalkyl,

and wherein Y optionally bears on carbon one or more R²⁸ substituents as hereinbefore defined.

(53) Y is selected from (1-4C)alkylene, cyclopropylene, cyclobutylene, cyclopentylene and cyclohexylene, and wherein adjacent carbon atoms in any (2-4C)alkylene chain within a Y substituent are optionally separated by the insertion into the chain of a group selected from O and N(R²⁷), wherein R²⁷ is hydrogen or (1-4C)alkyl,

and wherein Y optionally bears on carbon one or more R²⁸ substituents selected from halo, amino, hydroxy, (1-4C)alkyl, (1-4C)alkoxy, (1-4C)alkylamino and di-[(1-4C)alkyl]amino, and wherein R²⁸ optionally bears on carbon one or more substituents selected from halo, hydroxy, (1-4C)alkoxy and (3-6C)cycloalkyl.

(54) Y is selected from (1-4C)alkylene, and wherein adjacent carbon atoms in any (2-4C)alkylene chain within a Y substituent are optionally separated by the insertion into the chain of a group selected from O, N(R²⁷) and —C═C— and —C≡C— wherein R²⁷ is hydrogen or (1-4C)alkyl,

and wherein Y optionally bears on carbon one or more R²⁸ substituents selected from halo, amino, (1-4C)alkyl, (1-4C)alkylamino and di-[(1-4C)alkyl]amino, and wherein R²⁸ optionally bears one or more substituents selected from halo, hydroxy, (1-4C)alkoxy, amino, (1-4C)alkylamino, di-[(1-4C)alkyl]amino and (1-6C)cycloalkyl,

(55) Y is selected from (1-4C)alkylene, and wherein Y optionally bears on carbon one or more R²⁸ substituents selected from (1-3C)alkyl.
(56) Z is selected from a direct bond, O, S, NR²⁶—C═C—, —C≡C— and (1-4C)alkylene, wherein R²⁶ is hydrogen, (1-4C)alkyl or (3-6C)cycloalkyl, and wherein Z optionally bears on carbon one or more R²⁸ substituents wherein R²⁸ is as hereinbefore defined.
(57) Z is selected from a direct bond and N(R²⁶), wherein R²⁶ is hydrogen, (1-4C)alkyl or (3-6C)cycloalkyl, and wherein Z optionally bears on carbon one or more R²⁸ substituents selected from halo, amino, hydroxy, (1-4C)alkyl, (1-4C)alkoxy, (1-4C)alkylamino and di-[(1-4C)alkyl]amino, and wherein R²⁸ optionally bears on carbon one or more substituents selected from halo, hydroxy, (1-4C)alkoxy and (3-6C)cycloalkyl.
(58) Z is selected from a direct bond and N(R²⁶), wherein R²⁶ is hydrogen or (1-3C)alkyl.
(59) X and Z are independently selected from a direct bond, N(R²⁶), O and S (suitably a direct bond and NR²⁶, wherein R²⁶ is hydrogen or (1-4C)alkyl;

Y is selected from (1-4C)alkylene, and wherein Y optionally bears on carbon one or more R²⁸ substituents selected from (1-4C)alkyl.

(60) X is selected from a direct bond and O;

Y is selected from (1-4C)alkylene (suitably (2-4C)alkylene), and wherein Y optionally bears on carbon one or more R²⁸ substituents selected from (1-3C)alkyl; and

Z is selected from a direct bond and NR²⁶, wherein R²⁶ is hydrogen or (1-3C)alkyl.

(61) Y is (1-6C)alkylene (suitably Y is (2-6C)alkylene and particularly Y is (2-4C)alkylene).
(62) X is oxygen and Y is a (1-6C)alkylene (suitably Y is (2-6C)alkylene and particularly Y is (2-4C)alkylene).
(63) X is oxygen, Y is a (1-6C)alkylene (suitably Y is (2-6C)alkylene and particularly Y is (2-4C)alkylene) and Z is a direct bond.
(64) X is a direct bond, Y is a (1-6C)alkylene (suitably Y is (2-6C)alkylene and particularly Y is (2-4C)alkylene) and Z is selected from a direct bond and NR²⁶, wherein R²⁶ is hydrogen or (1-3C)alkyl.
(65) X, Y and Z have any of the values defined herein, and the group —X—Y-Z- has a chain length of from 3 to 6 atoms, for example 3, 4 or 5 atoms. Particularly when Z is NR²⁶ (for example NH), then the group —X—Y-Z- has a chain length of 5 atoms and when Z is not NR²⁶, then the group —X—Y-Z has a chain length of 3 atoms.
(66) The group —X—Y-Z is selected from —(CH₂)₃—, —(CH₂)₄—, *-O—(CH₂)₂—, *-O—(CH₂)₃-*-(CH₂)₃—N(R²⁶)—, -*-(CH₂)₄—N(R²⁶)—, *-O—(CH₂)₂—N(R²⁶)— and *-O—(CH₂)₃—N(R²⁶)— (particularly the group —X—Y-Z is selected from —(CH₂)₃—, *-O—(CH₂)₂—, *-(CH₂)₄—N(R²⁶)— and *-O—(CH₂)₃—N(R²⁶)—, more particularly —X—Y-Z- is selected from —(CH₂)₃—, —(CH₂)₄—, *-(CH₂)₃—N(R²⁶)— and *-(CH₂)₄—N(R²⁶)—, still more particularly —X—Y-Z- is —(CH₂)₃—);

wherein * represents the point of attachment to the thienyl ring in formula (I), and R²⁶ is H or (1-3C)alkyl (suitably R²⁶ is H);

and wherein the group X-Z-Y optionally bears on carbon one or more substituent selected from hydroxy, (1-3C)alkyl, (1-3C)alkoxy, hydroxy-(1-3C)alkyl, (1-3C)alkoxy-(1-3C)alkyl, hydroxy-(2-3C)alkoxy and (1-3C)alkoxy(2-3C)alkoxy (for example X—Y-Z optionally bears on carbon 1 or 2 (1-3C)alkyl substituents).

(67) The group —X—Y-Z- is selected from —(CH₂)—, —(CH₂)₂—, *-O—(CH₂)—, —O—(CH₂)₄-*-(CH₂)₂—N(R²⁶)—, —CH₂—O—CH₂—, *-CH₂—N(R²⁷)—(CH₂)—N(R²⁶)—C≡C—CH₂—N CC (CH₂)₂—N(R²⁶)-*-C═C—CH₂— and *-C═C—CH₂—N(R²⁶)— (particularly —X—Y-Z- is selected from —(CH₂)—, —(CH₂)₂—, *-(CH₂)₂—N(R²⁶)—, —CH₂—O—CH₂—, *-CH₂—N(R²⁷)—(CH₂)—N(R²⁶)—, *-C≡C—CH₂—N(R²⁶)—, *-C≡C—(CH₂)₂—N(R²⁶)-*-C═C—CH₂— and *-C═C—CH₂—N(R²⁶)—);

wherein * represents the point of attachment to the thienyl ring in formula (I), and R²⁶ and R²⁷ is H or (1-3C)alkyl (suitably R²⁶ and R²⁷ are both H);

and wherein the group X-Z-Y optionally bears on carbon one or more substituent selected from hydroxy, (1-3C)alkyl, (1-3C)alkoxy, hydroxy-(1-3C)alkyl, (1-3C)alkoxy-(1-3C)alkyl, hydroxy-(2-3C)alkoxy and (1-3C)alkoxy(2-3C)alkoxy (for example X—Y-Z optionally bears on carbon 1 or 2 (1-3C)alkyl substituents).

(68) The group —X—Y-Z is selected from —(CH₂)—C(O)—N(R²⁶)-*, —(CH₂)₂—C(O)—N(R²⁶)*-(CH₂)₃—C(O)—N(R²⁶)-*-(CH₂)—N(R²⁶)C(O)-*, —(CH₂)₂—N(R²⁶)C(O)-* and —(CH₂)₃—N(R²⁶)C(O)—,

wherein * represents the point of attachment to the thienyl ring in formula (I), and R²⁶ is H or (1-3C)alkyl (suitably R²⁶ is H);

and wherein the group X-Z-Y optionally bears on carbon one or more substituent selected from hydroxy, (1-3C)alkyl, (1-3C)alkoxy, hydroxy-(1-3C)alkyl, (1-3C)alkoxy-(1-3C)alkyl, hydroxy-(2-3C)alkoxy and (1-3C)alkoxy(2-3C)alkoxy (for example X—Y-Z optionally bears on carbon 1 or 2 (1-3C)alkyl substituents).

(69) The group —X—Y-Z is selected from —(CH₂)₃—, *-O—(CH₂)₂— and *-O—(CH₂)₃—NH—;

wherein * represents the point of attachment to the thienyl ring in formula (I);

and wherein the group X-Z-Y optionally bears on carbon one or more substituent selected from (1-3C)alkyl.

(70) The group —X—Y-Z is —(CH₂)₃—, and wherein the group X-Z-Y optionally bears on carbon one or more substituent selected from (1-3C)alkyl. Particularly —X—Y-Z is —(CH₂)₃—.
(71) The group —X—Y-Z is *-O—(CH₂)₂; wherein * represents the point of attachment to the thienyl ring in formula (I), and wherein the group X-Z-Y optionally bears on carbon one or more substituent selected from (1-3C)alkyl.
(72) The group —X—Y-Z is *-O—(CH₂)₃NH—; wherein * represents the point of attachment to the thienyl ring in formula (I), and wherein the group X-Z-Y optionally bears on carbon one or more substituent selected from (1-3C)alkyl.
(74) The group —X—Y-Z is *-(CH₂)₄NH—; wherein * represents the point of attachment to the thienyl ring in formula (I), and wherein the group X-Z-Y optionally bears on carbon one or more substituent selected from (1-3C)alkyl.
(75) R⁶ is a 5 or 6 membered monocyclic heteroaryl or a 9-, -10 or 11- (suitably 9- or 10-) membered bicyclic heteroaryl, which heteroaryl contains at least one —N═ ring atom and optionally 1 or more additional heteroatoms selected from O, S and N,

wherein R⁶ is linked to the group Z by a carbon atom in R⁶,

and wherein R⁶ optionally bears on carbon one or more R³¹ substituents,

and wherein if R⁶ contains an —NH— moiety, the nitrogen of said moiety optionally bears a group selected from R³⁵

and wherein:

(i) R⁶ is a 9-, -10 or 11- (suitably 9- or 10-) membered bicyclic fused heteroaryl which contains at least one unsubstituted —NH— ring member in addition to the —N═ ring atom in R⁶, wherein the —NH— and ═N— group in R⁶ are attached to the same bridgehead ring atom at a junction of the two fused rings in R⁶; or

(ii) R⁶ is substituted in an ortho position to the —N═ atom in R⁶ by an —NHR^{31a} group; or

(iii) Z is NH and R⁶ is attached to Z by a ring carbon atom in an ortho position to the —N═ atom in R⁶;

and wherein the group Z-R⁶ has a pKa of greater than or equal to 6, such as from 6 to 9;

wherein R³¹, R³⁵ and R^{31a} are as defined above.

(76) R⁶ is linked to Z by a ring carbon atom in R⁶ and R⁶ is selected from any one of (a) to (f):

(a) imidazole which is substituted in an ortho position to the —N═ of the imidazole ring by —NHR^{31a};

(b) imidazole fused to: (bi) a monocyclic 6-membered aromatic, (bii) a monocyclic 5- or 6-membered heteroaromatic, (biii) a 3 to 7-membered heterocyclic or (biv) a (3-6C)cycloalkane ring,

and wherein R⁶ is substituted in the ortho position to the —N═ of the imidazole ring by —NHR^{31a};

(c) imidazole fused to a 5-, 6 or 7-membered heterocyclic, or to a monocyclic 5- or 6-membered heteroaromatic which heterocyclic or heteroaromatic contains at least one unsubstituted —NH— ring member and optionally contains 1 or 2 additional hetero atoms selected from O, S and N, and wherein the unsubstituted —NH— of the heterocyclic or heteroaromatic ring and the ═N— of the imidazole in R⁶ are attached to the same bridgehead ring atom at a junction of the two fused rings;

(d) pyridine which is substituted in an ortho position to the —N═ of the pyridine ring by —NHR^{31a};

(e) pyridine fused to (bi) a monocyclic 6-membered aromatic, (bii) a monocyclic 5- or 6-membered heteroaromatic, (biii) a 3 to 7-membered heterocyclic or (biv) a (3-6C)cycloalkane ring,

and wherein R⁶ is substituted in an ortho position to the —N═ of the pyridine ring by —NHR^{31a}; and

(f) pyridine fused to a 5-, 6 or 7-membered heterocyclic, or to a monocyclic 5- or 6-membered heteroaromatic which heterocyclic or heteroaromatic contains at least one unsubstituted —NH— ring member and optionally contains 1 or 2 additional hetero atoms selected from O, S and N, and wherein the unsubstituted —NH— of the heterocyclic ring and the ═N— of the pyridine ring in R⁶ are attached to the same bridgehead ring atom at a junction of the two fused rings;

or the group Z is NH, R⁶ is attached to Z by a carbon atom that is ortho to a —N═ ring atom in R⁶ and R⁶ is selected from any one of (i) to (iv):

(i) imidazole;

(ii) imidazole fused to a 6-membered monocyclic aromatic, a 3- to 7-membered monocyclic heteroaromatic or heterocyclic ring or to a (3-6C)cycloalkane ring;

(iii) pyridine; and

(iv) pyridine fused to a 6-membered monocyclic aromatic, a 3- to 7-membered monocyclic heteroaromatic, a heterocyclic ring or a (3-6C)cycloalkane ring;

and wherein R⁶ optionally bears on carbon one or more R³¹ substituents,

and wherein if R⁶ contains an —NH— ring member, the nitrogen of said —NH— group optionally bears a group selected from R³⁵ (provided said —NH— group is not specified to be an unsubstituted —NH— above);

wherein R³¹, R^{31a} and R³⁵ are as hereinbefore defined. Suitably in this embodiment when X is O and Z is N(R²⁶), then R⁶ is not benzimidazolyl, particularly R⁶ is not benzimidazol-2-yl. Suitably R⁶ is linked to Z by a carbon atom in an aromatic ring in R⁶.

(77) R⁶ is linked to Z by a ring carbon atom in R⁶ and is selected from selected from any one of (a) to (f):

(a) imidazole which is substituted in an ortho position to the —N═ of the imidazole by —NHR^{31a};

(b) imidazole fused to a benzene, pyridine, pyrimidine, pyrazine, pyridazine or cyclohexane ring, and wherein R⁶ is substituted in an ortho position to the —N═ of the imidazole by —NHR^{31a};

(c) imidazole fused to a heterocyclic ring selected from morpholine, piperidine and piperazine, which heterocyclic ring carries at least one unsubstituted —NH— ring member, and wherein the unsubstituted —NH— of the heterocyclic ring and the ═N— of the imidazole ring in R⁶ are attached to the same bridgehead ring atom at a junction of the two fused rings;

(d) pyridine which is substituted in an ortho position to the —N═ of the pyridine by —NHR^{31a};

(e) pyridine fused to a benzene, 1,3-oxazole, isoxazole, furan, thiophene, 1,3-thiazole, isothiazole, pyrrole, pyridine, pyrimidine, pyrazine, cyclopentane or cyclohexane ring, and wherein R⁶ is substituted in an ortho position to the —N═ of the pyridine by —NHR^{31a}; and

(f) pyridine fused to a heterocyclic ring selected from morpholine, piperidine and piperazine, which heterocyclic ring carries at least one unsubstituted —NH— ring member, and wherein the unsubstituted —NH— of the heterocyclic ring and the ═N— of the imidazole ring in R⁶ are attached to the same bridgehead ring atom at a junction of the two fused rings;

or the group Z is NH, R⁶ is attached to Z by a carbon atom that is ortho to a —N═ ring atom in R⁶ and R⁶ is selected from one of (i) to (iv):

(i) imidazole;

(ii) imidazole fused to a benzene, pyridine, pyrimidine, pyrazine, pyridazine or cyclohexane ring;

(iii) pyridine; and

(iv) pyridine fused to a benzene, 1,3-oxazole, isoxazole, furan, thiophene, 1,3-thiazole, isothiazole, pyrrole, pyridine, pyrimidine, pyrazine, cyclopentane or cyclohexane ring;

and wherein R⁶ optionally bears on carbon one or more R³¹ substituents,

and wherein if R⁶ contains an —NH— ring member, the nitrogen of said —NH— group optionally bears a group selected from R³⁵ (provided said —NH— group is not specified to be an unsubstituted —NH— above);

wherein R³¹, R^{31a} and R³⁵ are as hereinbefore defined. Suitably in this embodiment when X is O and Z is N(R²⁶), then R⁶ is not benzimidazolyl, particularly R⁶ is not benzimidazol-2-yl. Suitably R⁶ is linked to Z by a carbon atom in an aromatic ring in R⁶.

(78) R⁶ is as defined in any one of (93) to (95) wherein R⁶ is linked to the group —X—Y-Z- by a ring carbon atom, which ring carbon atom is ortho to a —N═ atom in R⁶.
(79) R⁶ is imidazol-2-yl, which is substituted at the 5-position on the imidazolyl ring by —NHR^{31a} or R⁶ is pyridin-2-yl, which is substituted at the 6-position on the pyridyl ring by —NHR^{31a};

and wherein R⁶ optionally bears on carbon one or more R³¹ substituents,

and wherein the —NH— of the imidazol-2-yl ring optionally bears a group selected from R³⁵;

wherein R³¹, R^{31a} and R³⁵ are as hereinbefore defined.

(80) Z is NH and R⁶ is benzimidazol-2-yl and wherein R⁶ optionally bears on carbon one or more R³¹ substituents,

and wherein the —NH— of the benzimidazol-2-yl ring optionally bears a group selected from R³⁵;

wherein R³¹ and R³⁵ are as hereinbefore defined.
(81) R⁶ is as defined in any one of (75) to (80) wherein and wherein R⁶ optionally bears on carbon one or more R³¹ substituents selected from halo, amino, (1-4C)alkyl, (1-4C)alkoxy, (1-4C)alkylsulfonyl, (1-4C)alkylamino and di-[(1-4C)alkyl]amino,

or from a group of the formula:

—X⁸—R³²

wherein X⁸ is a direct bond, O or N(R³³), wherein R³³ is hydrogen or (1-4C)alkyl, and R³² is hydroxy-(1-4C)alkyl, (1-4C)alkoxy-(1-4C)alkyl, amino-(1-4C)alkyl, (1-4C)alkylamino-(1-4C)alkyl and di-[(1-4C)alkyl]amino-(1-4C)alkyl,

or from a group of the formula:

—X⁹-Q⁶

wherein X⁹ is a direct bond, O or N(R³⁴), wherein R³⁴ is hydrogen or (1-4C)alkyl, and Q⁶ is (3-6C)cycloalkyl or (3-6C)cycloalkyl-(1-4C)alkyl,

and wherein if R⁶ contains an —NH— ring member, the nitrogen of said —NH— group optionally bears an R³⁵ group (provided said —NH— group is not specified to be an unsubstituted —NH— above);

R³⁵ is selected from (1-4C)alkyl, hydroxy-(2-4C)alkyl, (1-4C)alkoxyalkyl, amino-(2-4C)alkyl, (1-4C)alkylamino-(2-4C)alkyl, di-[(1-4C)alkyl]amino-(2-4C)alkyl, (3-6C)cycloalkyl and (3-6C)cycloalkyl-(1-4C)alkyl,

and wherein R^{31a} is selected from hydrogen, (1-6C)alkyl, (2-8C)alkenyl, (2-8C)alkynyl, halo-(1-4C)alkyl, hydroxy-(2-4C)alkyl, (1-4C)alkoxy-(2-4C)alkyl, amino-(2-4C)alkyl, (1-4C)alkylamino-(2-4C)alkyl, di-[(1-4C)alkyl]amino-(2-4C)alkyl, (3-6C)cycloalkyl and (3-6C)cycloalkyl-(1-4C)alkyl, and wherein any (3-6C)cycloalkyl in R^{31a} optionally bears 1 or more (for example 1 or 2) (1-4C)alkyl substituents. Suitably R³¹ is selected from halo and (1-4C)alkyl.

(82) R⁶ is as defined in any one of (75) to (81) wherein and wherein R⁶ optionally bears on carbon one or more R³¹ substituents selected from amino, (1-4C)alkyl, (1-4C)alkoxy, (1-4C)alkylamino and di-[(1-4C)alkyl]amino,

or from a group of the formula:

—X⁹-Q⁶

wherein X⁹ is a direct bond, O or N(R³⁴), wherein R³⁴ is hydrogen or (1-4C)alkyl, and Q⁶ is (3-6C)cycloalkyl or (3-6C)cycloalkyl-(1-4C)alkyl,

and wherein if R⁶ contains an —NH— ring member, the nitrogen of said —NH— group optionally bears an R³⁵ group (provided said —NH— group is not specified to be an unsubstituted —NH— above);

R³⁵ is selected from (1-4C)alkyl, (3-6C)cycloalkyl and (3-6C)cycloalkyl-(1-4C)alkyl,

and wherein R^{31a} is selected from hydrogen, (1-4C)alkyl, amino-(2-4C)alkyl, (1-4C)alkylamino-(2-4C)alkyl, di-[(1-4C)alkyl]amino-(2-4C)alkyl, (3-6C)cycloalkyl and (3-6C)cycloalkyl-(1-4C)alkyl, and wherein any (3-6C)cycloalkyl in R^{31a} optionally bears 1 or more (for example 1 or 2) (1-4C)alkyl substituents. Suitably R³¹ is selected from halo and (1-3C)alkyl, for example fluoro, methyl or ethyl.

(83) R⁶ is a heteroaryl as described in any of (75) to (82), wherein if any bicyclic heteroaryl is partially aromatic, said partially aromatic bicyclic heteroaryl is attached to the group Z by carbon atom in the aromatic ring of the partially aromatic bicyclic heteroaryl.
(84) R⁶ is a heteroaryl as described in any of (75) to (83), wherein R⁶ is linked to the group —X—Y-Z- by a ring carbon atom, which ring carbon atom is ortho to a ring —N═ atom in R⁶.
(85) R⁶ is selected from:

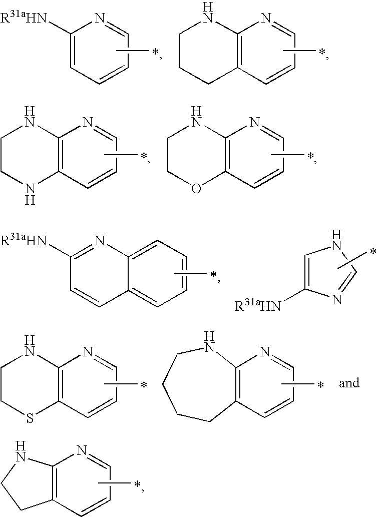

wherein * indicates the point of attachment of R⁶ to the group Z in formula I, and wherein R^{31a} is as hereinbefore defined and the R⁶ groups are optionally substituted as defined herein, for example in any one of (81) to (82) above.

In (85) above the bond indicates the ring that is bonded to the group Z in formula (I). For example the group

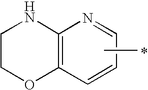

encompasses for example:

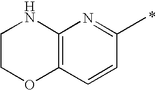

but not

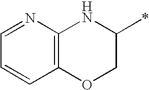

(86) R⁶ is selected from:

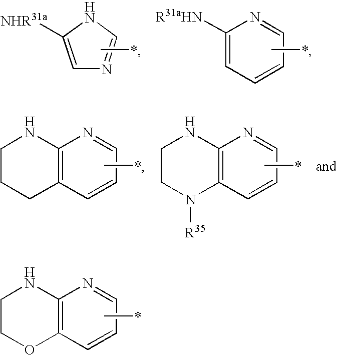

wherein * indicates the point of attachment of R⁶ to the group Z in formula I, and R⁶ is attached to the group —X—Y-Z- by a carbon atom in an aromatic ring of R⁶;

R^{31a} is as hereinbefore defined, for example as in any of (81) to (82) above;
R³⁵ is as hereinbefore defined, for example R³⁵ is (1-4C)alkyl, (3-6C)cycloalkyl and (3-6C)cycloalkyl-(1-4C)alkyl;

and wherein the R⁶ groups are optionally substituted on a ring carbon by one of more R³¹ as hereinbefore defined, for example as described in any one of (81) to (82) above.

(87) Z is NH and the group R⁶-Z- is selected from:

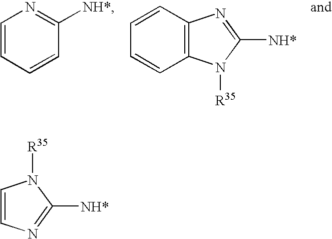

wherein * indicates the point of attachment of R⁶-Z- to Y in formula I,

R³⁵ is as hereinbefore defined, for example R³⁵ is hydrogen, (1-4C)alkyl, (3-6C)cycloalkyl and (3-6C)cycloalkyl-(1-4C)alkyl;

and wherein the R⁶ groups are optionally substituted on a ring carbon by one of more R³¹ as hereinbefore defined, for example as described in any one of (81) to (82) above.

(88) R⁶ is selected from:

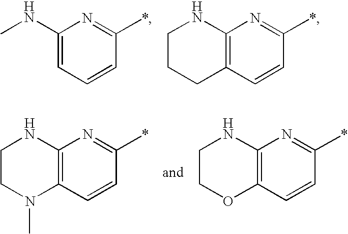

or the group R⁶-Z- is selected from:

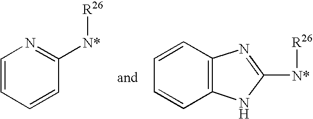

wherein R²⁶ is as hereinbefore defined (for example R²⁶ is hydrogen); and
wherein * indicates the point of attachment of R⁶ to the group Z or R⁶-Z- to Y in formula I, and wherein the R⁶ groups are optionally substituted as defined in any one of (81) to (82).
(89) R⁶ is selected from:

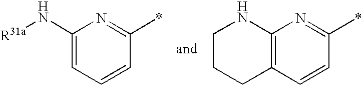

wherein R^{31a} is as hereinbefore defined. Particularly R^{31a} is selected from hydrogen (1-3C)alkyl, (3-6C)cycloalkyl, or (3-6C)cycloalkyl-(1-3C)alkyl, more particularly R^{31a} is (1-3C)alkyl, cyclopropyl or cyclopropylmethyl, for example R^{31a} is methyl;

* indicates the point of attachment of R⁶ to the group Z in formula I;

and wherein the R⁶ groups are optionally substituted on carbon by R³¹ as defined herein for example in any one of (81) or (82). In a particular embodiment R⁶ is not substituted by R³¹. In another particular embodiment R⁶ is 5,6,7,8-tetrahydro-1,8-naphthyridin-2-yl which optionally bears at the 6-, 7- or 8-positions one or more R³¹ substituents as hereinbefore defined, for example as defined in any one of (81) or (82), particularly R³¹ is (1-3C)alkyl, (3-6C)cycloalkyl and (3-6C)cycloalkyl-(1-3C)alkyl, (for example R³¹ is methyl or methoxy). Suitably however, R⁶ is not substituted by R³¹. (90) R⁶ is selected from:
(i) pyridin-2-yl, which is substituted at the 6-position by —NHR^{31a};
(ii) 3,4-dihydro-2H-pyrido[3,2-b][1,4]oxazin-6-yl;
(iii) quinolin-7-yl, which is substituted at the 6-position by —NHR^{31a};
(iv) 1,2,3,4-tetrahydropyrido[2,3-b]pyrazin-6-yl; and
(v) 5,6,7,8-tetrahydro-1,8-naphthyridin-2-yl;

or Z is NH and the group R⁶-Z- is selected from 1H-benzimidazol-2-ylamino and pyridin-2-ylamino;

wherein R^{31a} is as hereinbefore defined, for example as defined in (81) or (82) above, more particularly R^{31a} is (1-3C)alkyl, cyclopropyl or cyclopropylmethyl, for example R^{31a} is methyl;

and wherein the R⁶ groups are optionally substituted on carbon by R³¹ wherein R³¹ is selected from (1-3C)alkyl, (3-6C)cycloalkyl, (3-6C)cycloalkyl-(1-3C)alkyl and (1-3Calkoxy) such as methyl or methoxy.

(91) R⁶ is selected from 6-aminopyridin-2-yl, 6-(cyclopentylamino)pyridin-2-yl, 6-(cyclopropylamino)pyridin-2-yl, 6-[(cyclopropylmethyl)amino]pyridin-2-yl, 3,4-dihydro-2H-pyrido[3,2-b][1,4]oxazin-6-yl, 6-{[2-(dimethylamino)ethyl]amino}pyridin-2-yl, 6-{[2-(methoxyethyl)amino]pyridin-2-yl, 5-methoxy-6-(methylamino)pyridin-2-yl, 6-(dimethylamino)pyridin-2-yl, 6-(methylamino)pyridin-2-yl, 2-(methylamino)quinolin-7-yl, 1-methyl-1,2,3,4-tetrahydropyrido[2,3-b]pyrazin-6-yl, 4-methyl-5,6,7,8-tetrahydro-1,8-naphthyridin-2-yl, 5-methyl-5,6,7,8-tetrahydro-1,8-naphthyridin-2-yl, 7-methyl-5,6,7,8-tetrahydro-1,8-naphthyridin-2-yl and 5,6,7,8-tetrahydro-1,8-naphthyridin-2-yl;

or Z is NH and the group R⁶-Z- is selected from:

1H-benzimidazol-2-ylamino, 4-ethylpyridin-2-ylamino, 5-methylpyridin-2-ylamino, 4-methylpyridin-2-ylamino, 6-methylpyridin-2-ylamino, 3-methylpyridin-2-ylamino, 4,6-dimethylpyridin-2-ylamino, 4-methoxypyridin-2-yl)amino, 5-methoxypyridin-2-ylamino, and pyridin-2-ylamino.
(92) R⁶ is selected from:

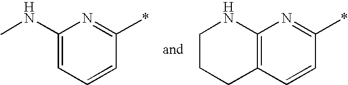

wherein * indicates the point of attachment of R⁶ to the group Z in formula I;

and wherein the R⁶ groups are optionally substituted on carbon by R³¹ as defined hereinbefore, for example R³¹ is selected from (1-3C)alkyl, (3-6C)cycloalkyl, (3-6C)cycloalkyl-(1-3C)alkyl and (1-3Calkoxy) such as methyl or methoxy. Suitably, however, these R⁶ groups are not substituted by R³¹. Accordingly a particular R⁶ is

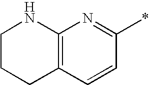

(93) R⁶ is as defined in (88) to (92) and the group X—Y-Z- is as defined in any one of (66) to (74).

### (94) R⁶ is

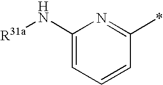

wherein R^{31a} is selected from hydrogen (1-3C)alkyl, (3-6C)cycloalkyl, or (3-6C)cycloalkyl-(1-3C)alkyl, more particularly R^{31a} is (1-3C)alkyl, cyclopropyl or cyclopropylmethyl, for example R^{31a} is methyl;
* indicates the point of attachment of R⁶ to the group Z in formula I; and the group —X—Y-Z- is —(CH₂)₃— or **-O—(CH₂)₂; wherein ** represents the point of attachment to the thienyl ring in formula (I). Particularly —X—Y-Z- is —(CH₂)₃—. More particularly —X—Y-Z- is **-O—(CH₂)₂—.

### (95) R⁶ is

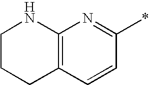

wherein * indicates the point of attachment of R⁶ to the group Z in formula I; and the group —X—Y-Z- is —(CH₂)₃— or **-O—(CH₂)₂—; wherein ** represents the point of attachment to the thienyl ring in formula (I). Particularly —X—Y-Z- is —(CH₂)₃—. More particularly —X—Y-Z- is **-O—(CH₂)₂—.

### (96) R⁶ is

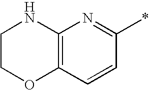

wherein * indicates the point of attachment of R⁶ to the group Z in formula I; and the group —X—Y-Z- is —(CH₂)₃— or **-O—(CH₂)₂; wherein ** represents the point of attachment to the thienyl ring in formula (I). Particularly —X—Y-Z- is —(CH₂)₃—. More particularly —X—Y-Z- is **-O—(CH₂)₂—.
(97) X, Y, Z and R⁶ have any of the values defined herein and the chain length between the thienyl ring and a ring —N═ in R⁶ is 4 or 5, particularly 5 atoms long;

or when Z is NH and R⁶ is attached to Z by a carbon atom that is in an ortho position to a —N═ ring atom in R⁶, then the chain length of the group —X—Y-Z- is 4 or 5 (particularly 5) atoms long.

(98) X, Y, Z and R⁶ have any of the values defined herein, and the chain length between the thienyl ring and a ring —N═ in R⁶ is 4 or 5 (particularly 5) atoms long.
(99) X^{a} is oxygen.

In an embodiment of the invention there is provided a compound of the formula I which is of the formula IA:

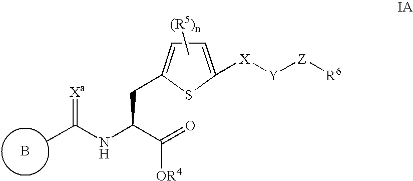

wherein:

n, B, R⁴, R⁵, R⁶, X^{a}, X, Y and Z have any of the values as hereinbefore defined;

or a pharmaceutically acceptable salt thereof.

Particular compounds of the Formula IA are those wherein X^{a} is oxygen.

In a particular embodiment there is provided a compound of formula (IA) or a pharmaceutically acceptable salt thereof, wherein:

B is as defined in any on of paragraphs (1) to (33) above, provided that:
(i) B bears at least 1 halo substituent; and
(ii) B is substituted in an ortho position to the group C(X^{a}) by a substituent selected from chloro, bromo, (1-3C)alkyl, cyclopropyl and halo-(1-3C)alkyl (particularly B is substituted in an ortho position relative to C(X^{a}) by a substituent selected from methyl, ethyl and chloro, more particularly chloro);

X^{a} is oxygen;

R⁴ is H;

n is 0, 1, or 2 and R⁵ is as defined in any one of paragraphs (39) to (45) (particularly n is 0 or 1, more particularly n is 0);

the group —X—Y-Z- is as defined in any one of paragraphs (66) to (74) (particularly —X—Y-Z- is selected from is selected from —(CH₂)₃— and *-O—(CH₂)₂—, (more particularly —X—Y-Z- is —(CH₂)₃—, still more particularly —X—Y-Z- is *-O—(CH₂)₂—); wherein * represents the point of attachment to the thienyl ring in formula (IA); and

R⁶ is as defined in any one of paragraphs (75) to (92) above.

In this embodiment B is suitably selected from isoxazolyl, 1,3-oxazolyl, thienyl, furyl, 1,3 thiazolyl, pyrrolyl, pyrazolyl, pyridyl, indolyl and benzofuranyl, and wherein B is attached to the C(X^{a}) group by a ring carbon atom (particularly B is selected from isoxazolyl, 1,3-oxazolyl, thienyl, furyl, 1,3-thiazolyl, pyrrolyl, pyrazolyl, pyridyl and benzofuranyl, more particularly B is selected from isoxazolyl, still more particularly B is isoxazol-4-yl,);

and wherein B is substituted in an ortho position to the group C(X^{a}) by a substituent selected from chloro, methyl and ethyl;

and wherein B optionally bears on carbon 1 or 2 substituents selected from halo, trifluoromethyl (1-3C)alkyl and (1-4C)alkoxy.

For example B is selected from 3-chloro-2-thienyl, 3-chloro-4-methyl-2-thienyl, 2,5-dichloro-3-thienyl, 3-chloropyridin-2-yl, 3,6-dichloropyridin-2-yl, 3,5-dichloropyridin-2-yl, 3-chloro-5-(trifluoromethyl)pyridin-2-yl, 2-chloro-6-methylpyridi-3-yl, 2-chloropyridin-3-yl, 3,5-dichloropyridin-4-yl and 2,6-dichloropyridin-3-yl. Alternatively B is 3,5-dichloropyridin-4-yl or 3,5-dimethylisoxazol-4-yl, for example B is 3,5-dimethylisoxazol-4-yl.

In another particular embodiment there is provided a compound of formula (IA) or a pharmaceutically acceptable salt thereof, wherein:

B is selected from isoxazolyl, 1,3-oxazolyl, thienyl, furyl, 1,3-thiazolyl, pyrrolyl, pyrazolyl, pyridyl, indolyl and benzofuranyl, and wherein B is attached to the C(X^{a}) group by a ring carbon atom (particularly B is selected from isoxazolyl, 1,3-oxazolyl, thienyl, furyl, 1,3 thiazolyl, pyrrolyl, pyrazolyl, pyridyl and benzofuranyl, more particularly B is selected from isoxazolyl and pyridyl, still more particularly B is isoxazol-4-yl or 4-pyridyl, for example B is isoxazol-4-yl);

and wherein B is substituted in an ortho position to the group C(X^{a}) by a substituent selected from chloro, methyl and ethyl;

and wherein B optionally bears on carbon 1 or 2 substituents selected from halo, trifluoromethyl (1-3C)alkyl and (1-4C)alkoxy, provided that B carries at least 1 halo substituent;

X^{a} is oxygen;

R⁴ is H;

n is 0, 1, or 2 and each R⁵, which may be the same or different, is selected from halo, hydroxy, amino, (1-4C)alkyl, (1-4C)alkoxy(1-4C)alkylamino and di-[(1-4C)alkyl]amino,

and wherein R⁵ optionally bears on carbon one or more R²⁴ substituents selected from halo, hydroxy, amino, (1-4C)alkoxy, (1-4C)alkylamino and di-[(1-4C)alkyl]amino (particularly n is 0 or 1, more particularly n is 0);

the group —X—Y-Z- is selected from —(CH₂)₃— and *-O—(CH₂)₂ (particularly —X—Y-Z- is —(CH₂)₃—, more particularly —X—Y-Z- is *-O—(CH₂)₂—); wherein * represents the point of attachment to the thienyl group in formula (IA); and
R⁶ is selected from:

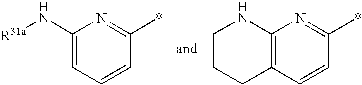

wherein R^{31a} is hydrogen or (1-3C)alkyl (particularly R^{31a} is (1-3C)alkyl, such as methyl); and
* indicates the point of attachment of R⁶ to the group Z in formula I.

In another particular embodiment there is provided a compound of formula (IA) or a pharmaceutically acceptable salt thereof, wherein:

B is selected from isoxazolyl, 1,3-oxazolyl, thienyl, furyl, 1,3-thiazolyl, pyrrolyl, pyrazolyl, pyridyl, indolyl and benzofuranyl, and wherein B is attached to the C(X^{a}) group by a ring carbon atom (particularly B is selected from isoxazolyl, 1,3-oxazolyl, thienyl, furyl, 1,3 thiazolyl, pyrrolyl, pyrazolyl, pyridyl and benzofuranyl, more particularly B is selected from isoxazolyl and pyridyl, still more particularly B is 4-pyridyl or isoxazol-4-yl, for example B is isoxazol-4-yl);

and wherein B is substituted in an ortho position to the group C(X^{a}) by a substituent selected from chloro, methyl and ethyl;

and wherein B optionally bears on carbon 1 or 2 substituents selected from halo, trifluoromethyl (1-3C)alkyl and (1-4C)alkoxy;

X^{a} is oxygen;

R⁴ is H;

n is 0, 1, or 2 and each R⁵, which may be the same or different, is selected from halo, hydroxy, amino, (1-4C)alkyl, (1-4C)alkoxy(1-4C)alkylamino and di-[(1-4C)alkyl]amino,

and wherein R⁵ optionally bears on carbon one or more R²⁴ substituents selected from halo, hydroxy, amino, (1-4C)alkoxy, (1-4C)alkylamino and di-[(1-4C)alkyl]amino (particularly n is 0 or 1, more particularly n is 0);

the group —X—Y-Z- is selected from —(CH₂)₃— and *-O—(CH₂)₂ (particularly —X—Y-Z- is —(CH₂)₃—, more particularly —X—Y-Z- is *-O—(CH₂)₂—); wherein * represents the point of attachment to the thienyl group in formula (IA); and

R⁶ is selected from:

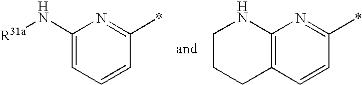

wherein R^{31a} is hydrogen or (1-3C)alkyl (particularly R^{31a} is (1-3C)alkyl, such as methyl); and
* indicates the point of attachment of R⁶ to the group Z in formula I.

In another particular embodiment there is provided compounds of formula (IA) or a pharmaceutically acceptable salt thereof, wherein:

B is selected from isoxazolyl, 1,3-oxazolyl, thienyl, furyl, 1,3 thiazolyl, pyrrolyl, pyrazolyl, pyridyl, indolyl and benzofuranyl, and wherein B is attached to the C(X^{a}) group by a ring carbon atom (particularly B is selected from isoxazolyl, 1,3-oxazolyl, thienyl, furyl, 1,3 thiazolyl, pyrrolyl, pyrazolyl, pyridyl and benzofuranyl, more particularly B is selected from isoxazolyl and pyridyl, still more particularly B is 4-pyridyl or isoxazol-4-yl, for example B is isoxazol-4-yl);

and wherein B is substituted in an ortho position to the group C(X^{a}) by a substituent selected from chloro, methyl and ethyl;

and wherein B optionally bears on carbon 1 or 2 substituents selected from halo, trifluoromethyl (1-3C)alkyl and (1-4C)alkoxy;

X^{a} is oxygen;

R⁴ is H;

n is 0 or 1 and R⁵ is selected from halo, hydroxy, (1-4C)alkyl and (1-4C)alkoxy (suitably n=0);

the group —X—Y-Z- is selected from —(CH₂)₃— and *-O—(CH₂)₂ (particularly —X—Y-Z- is —(CH₂)₃—, more particularly —X—Y-Z- is *-O—(CH₂)₂—); wherein * represents the point of attachment to the thienyl group in formula (IA); and

R⁶ is as defined in any one of paragraphs (75) to (92) above.

For example in this embodiment B is as defined in (32) or (33) above, for example B is selected from 3-chloro-2-thienyl, 3-methyl-2-thienyl, 3-chloro-4-methyl-2-thienyl, 2,5-dichloro-3-thienyl, 1-methyl-1H-pyrrol-2-yl, 3-methyl-1H-pyrazol-4-yl, 3,5-dimethyl-1H-pyrazol-4-yl, 1,3,5-trimethyl-1H-pyrazol-4-yl, 1-ethyl-3-methyl-1H-pyrazol-5-yl, 3-methyl-2-furoyl, 2-methyl-3-furoyl, 4-methyl-1,3-thiazol-5-yl, 2,4-dimethyl-1,3-thiazol-5-yl, 3-methylisoxazol-4-yl, 3,5-dimethylisoxazol-4-yl, 2,5-dimethyl-1,3-oxazol-4-yl, 4-methyl-1,3-oxazol-5-yl, 3-chloropyridin-2-yl, 3-methylpyridin-2-yl, 3,6-dichloropyridin-2-yl, 3,5-dichloropyridin-2-yl, 3-chloro-5-(trifluoromethyl)pyridin-2-yl, 2-chloro-6-methylpyridin-3-yl, 4-methylpyridin-3-yl, 2-methylpyridin-3-yl, 2-chloropyridin-3-yl, 3,5-dichloropyridin-4-yl, 3-methylpyridin-4-yl, 2-(ethylthio)pyridin-3-yl, 2,6-dichloropyridin-3-yl, 2,6-dimethoxypyridin-3-yl, and 3-methyl-1-benzofuran-2-yl. Particularly B is 3,5-dichloropyridin-4-yl or 3,5-dimethylisoxazol-4-yl, for example B is 3,5-dimethylisoxazol-4-yl.

In another embodiment of the invention there is provided a compound of the formula I of the formula (IB):

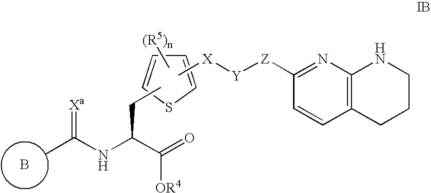

wherein:

n, R⁴, R⁵, X^{a}, X, Y and Z have any of the values as hereinbefore defined;

or a pharmaceutically acceptable salt thereof.

A particular compound of the formula (IB) is a compound of the formula (IB′):

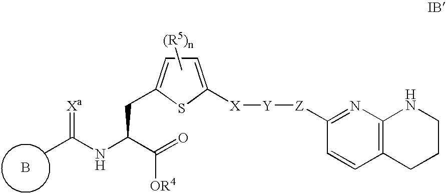

wherein:

n, R⁴, R⁵, X, Y and Z have any of the values as hereinbefore defined;

or a pharmaceutically acceptable salt thereof.

In another embodiment of the invention there is provided a compound of the formula I of the formula (IC):

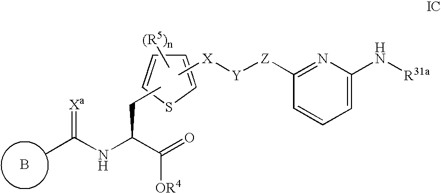

wherein:

R^{31a} is as hereinbefore defined, for example R^{31a} is selected from hydrogen (1-3C)alkyl, (3-6C)cycloalkyl, or (3-6C)cycloalkyl-(1-3C)alkyl, more particularly R^{31a} is (1-3C)alkyl, cyclopropyl or cyclopropylmethyl, for example R^{31a} is methyl; and

n, R⁴, R⁵, X^{a}, X, Y and Z have any of the values as hereinbefore defined;

or a pharmaceutically acceptable salt thereof.

A particular compound of the formula (IC) is a compound of the formula (IC′):

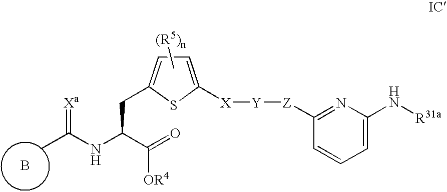

wherein:

R^{31a} is as hereinbefore defined, for example R^{31a} is selected from hydrogen (1-3C)alkyl, (3-6C)cycloalkyl, or (3-6C)cycloalkyl-(1-3C)alkyl, more particularly R^{31a} is (1-3C)alkyl, cyclopropyl or cyclopropylmethyl, for example R^{31a} is methyl; and

n, R⁴, R⁵, X, Y and Z have any of the values as hereinbefore defined;

or a pharmaceutically acceptable salt thereof.

In another embodiment of the invention there is provided a compound of the formula I of the formula (ID):

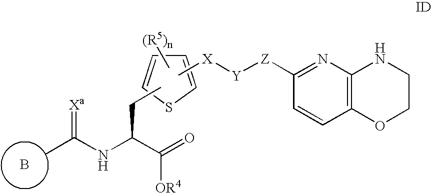

wherein:

n, B, R⁴, R⁵, X^{a}, X, Y and Z have any of the values as hereinbefore defined;

or a pharmaceutically acceptable salt thereof.

Particular compounds of the Formula ID are of the Formula ID′:

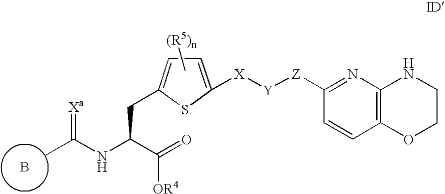

wherein:

n, B, R⁴, R⁵, X^{a}, X, Y and Z have any of the values as hereinbefore defined;

or a pharmaceutically acceptable salt thereof.

In a further embodiment, there is provided a compound of the formula (IB), (IB′), (IC), (IC′), (ID) or (ID′) or a pharmaceutically acceptable salt thereof, wherein:

R⁴ is hydrogen;

X^{a} is O;

n is 0, 1, or 2 and each R⁵, which may be the same or different, has any of the values defined herein, for example R⁵ is as defined in any one of (39) to (45) above, particularly R⁵ is selected from halo, hydroxy, amino, (1-4C)alkyl, (1-4C)alkoxy (1-4C)alkylamino and di-[(1-4C)alkyl]amino,

and wherein R⁵ optionally bears on carbon one or more R²⁴ substituents selected from halo, hydroxy, amino, (1-4C)alkoxy, (1-4C)alkylamino and di-[(1-4C)alkyl]amino (particularly n is 0 or 1, more particularly n is 0);

the group —X—Y-Z- is selected from —(CH₂)₃— and *-O—(CH₂)₂; wherein * represents the point of attachment to the thienyl group (a particular value for —X—Y-Z- is —(CH₂)₃— more particularly —X—Y-Z- is *-O—(CH₂)₂—), and wherein —X—Y-Z- optionally bears on carbon one or two (1-3C)alkyl substituents (suitably however, —X—Y-Z- is unsubstituted); and

B has any of the values defined herein. Particularly B is selected from isoxazolyl, 1,3-oxazolyl, thienyl, furyl, 1,3-thiazolyl, pyrrolyl, pyrazolyl, pyridyl and benzofuranyl, and wherein B is attached to the C(X^{a}) group by a ring carbon atom (more particularly B is thienyl, isoxazolyl or pyridyl, more particularly B is 2-thienyl, 4-pyridyl or isoxazol-4-yl, for example B is isoxazol-4-yl);

and wherein B is substituted in an ortho position to the group C(X^{a}) by a substituent selected from chloro, methyl and ethyl;

and wherein B optionally bears on carbon 1 or 2 substituents selected from halo, trifluoromethyl (1-3C)alkyl and (1-4C)alkoxy.

For example B in any of the compounds of the formulae (IB), (IB′), (IC), (IC′), (ID) or (ID′) is as defined in (32) or (33) above, for example B is selected from 3-chloro-2-thienyl, 3-methyl-2-thienyl, 3-chloro-4-methyl-2-thienyl, 2,5-dichloro-3-thienyl, 1-methyl-1H-pyrrol-2-yl, 3-methyl-1H-pyrazol-4-yl, 3,5-dimethyl-1H-pyrazol-4-yl, 1,3,5-trimethyl-1H-pyrazol-4-yl, 1-ethyl-3-methyl-1H-pyrazol-5-yl, 3-methyl-2-furoyl, 2-methyl-3-furoyl, 4-methyl-1,3-thiazol-5-yl, 2,4-dimethyl-1,3-thiazol-5-yl, 3-methylisoxazol-4-yl, 3,5-dimethylisoxazol-4-yl, 2,5-dimethyl-1,3-oxazol-4-yl, 4-methyl-1,3-oxazol-5-yl, 3-chloropyridin-2-yl, 3-methylpyridin-2-yl, 3,6-dichloropyridin-2-yl, 3,5-dichloropyridin-2-yl, 3-chloro-5-(trifluoromethyl)pyridin-2-yl, 2-chloro-6-methylpyridin-3-yl, 4-methylpyridin-3-yl, 2-methylpyridin-3-yl, 2-chloropyridin-3-yl, 3,5-dichloropyridin-4-yl, 3-methylpyridin-4-yl, 2-(ethylthio)pyridin-3-yl, 2,6-dichloropyridin-3-yl, 2,6-dimethoxypyridin-3-yl, and 3-methyl-1-benzofuran-2-yl. Particularly B is selected from 3-chloro-2-thienyl, 3,5-dichloropyridin-4-yl and 3,5-dimethylisoxazol-4-yl, more particularly B is 3,5-dichloropyridin-4-yl or 3,5-dimethylisoxazol-4-yl, for example B is 3,5-dimethylisoxazol-4-yl.

In a further embodiment, there is provided a compound of the formula (IB), (IB′), (IC), (IC′), (ID) or (ID′) or a pharmaceutically acceptable salt thereof, wherein:

R⁴ is hydrogen;

X^{a} is O;

n is 0, 1, or 2 and each R⁵, which may be the same or different, has any of the values defined herein, for example R⁵ is as defined in any one of (39) to (45) above, particularly R⁵ is selected from halo, hydroxy, amino, (1-4C)alkyl, (1-4C)alkoxy (1-4C)alkylamino and di-[(1-4C)alkyl]amino,

and wherein R⁵ optionally bears on carbon one or more R²⁴ substituents selected from halo, hydroxy, amino, (1-4C)alkoxy, (1-4C)alkylamino and di-[(1-4C)alkyl]amino (particularly n is 0 or 1, more particularly n is 0);

the group —X—Y-Z- is selected from —(CH₂)₃— and *-O—(CH₂)₂; wherein * represents the point of attachment to the thienyl group (particularly —X—Y-Z- is —(CH₂)₃— more particularly —X—Y-Z- is *-O—(CH₂)₂—) and wherein —X—Y-Z- optionally bears on carbon one or two (1-3C)alkyl substituents (suitably however, —X—Y-Z- is unsubstituted); and

B is of the formula BII:

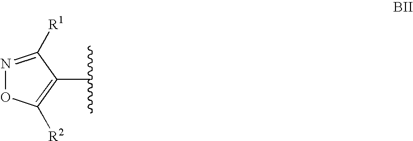

R¹ is selected from halo, (1-3C)alkyl, (1-3C)alkoxy, (1-3C)alkylthio, cyclopropyl, cyclobutyl, cyclopentyl, cyclopropyl-(1-2C)alkyl, cyclobutyl-(1-2C)alkyl and cyclopentyl-(1-2C)alkyl (particularly R¹ is selected from halo and (1-3C)alkyl such as fluoro, chloro, bromo, methyl and ethyl more particularly R¹ is selected from chloro and methyl, still more particularly R¹ is (1-3C)alkyl such as methyl); and

R² is selected from hydrogen, halo, (1-3C)alkyl, (1-3C)alkoxy, (1-3C)alkylthio, halo-(1-3C)alkyl, cyclopropyl, cyclobutyl, cyclopentyl, cyclopropyl-(1-2C)alkyl, cyclobutyl-(1-2C)alkyl and cyclopentyl-(1-2C)alkyl (for example R² is selected from hydrogen, fluoro, chloro, bromo, methyl, ethyl and trifluoromethyl; more particularly R² is not hydrogen, for example R² is (1-3C)alkyl, such as methyl).

Accordingly a particular value for B in the formulae (IB), (IB′), (IC), (IC′), (ID) or (ID′) is 3,5-dimethylisoxazol-4-yl.

In a further embodiment, there is provided a compound of the formula (IB), (IB′), (IC), (IC′), (ID) or (ID′) or a pharmaceutically acceptable salt thereof, wherein:

R⁴ is hydrogen;

X^{a} is O;

n is 0, 1, or 2 and each R⁵, which may be the same or different, has any of the values defined herein, for example R⁵ is as defined in any one of (39) to (45) above, particularly R⁵ is selected from halo, hydroxy, amino, (1-4C)alkyl, (1-4C)alkoxy (1-4C)alkylamino and di-[(1-4C)alkyl]amino,

and wherein R⁵ optionally bears on carbon one or more R²⁴ substituents selected from halo, hydroxy, amino, (1-4C)alkoxy, (1-4C)alkylamino and di-[(1-4C)alkyl]amino (particularly n is 0 or 1, more particularly n is 0);

the group —X—Y-Z- is selected from —(CH₂)₃— and *-O—(CH₂)₂; wherein * represents the point of attachment to the thienyl group (particularly —X—Y-Z- is —(CH₂)₃—, more particularly —X—Y-Z- is *-O—(CH₂)₂—) and wherein —X—Y-Z- optionally bears on carbon one or two (1-3C)alkyl substituents (suitably however, —X—Y-Z- is unsubstituted); and

B is of the formula BI:

BI:

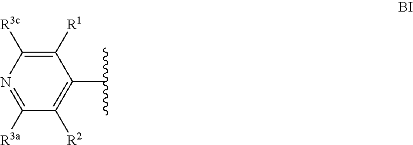

wherein:

R¹ is selected from halo (particularly fluoro, chloro or bromo), (1-3C)alkyl, (1-3C)alkoxy, (1-3C)alkylthio (for example R¹ is selected from chloro, bromo and (1-3C)alkyl);

R² is selected from hydrogen, (1-3C)alkyl, (1-3C)alkoxy, (1-3C)alkylthio, such as hydrogen, methyl, ethyl, fluoro, chloro and bromo (suitably however R² is not hydrogen); and

R^{3a} and R^{3c}, which may be the same or different, are hydrogen or have any of the values defined herein for R³, for example R^{3a} and R^{3c} are selected from hydrogen, halo (particularly fluoro or chloro, more particularly chloro), (1-3C)alkyl, (1-3C)alkoxy, (1-3C)thioalkyl and trifluoromethyl.

Accordingly a particular value for B in the formulae (IB), (IB′), (IC), (IC′), (ID) or (ID′) is 3,5-dichloropyridin-4-yl.

In a further embodiment, there is provided a compound of the formula (IB), (IB′), (IC), (IC′), (ID) or (ID′) or a pharmaceutically acceptable salt thereof, wherein:

R⁴ is hydrogen;

X^{a} is O;

n is 0,

the group —X—Y-Z- is *-O—(CH₂)₂; wherein * represents the point of attachment to the thienyl group, and wherein —X—Y-Z- optionally bears on carbon one or two (1-3C)alkyl substituents (suitably however, —X—Y-Z- is unsubstituted); and

B is selected from 3-chloro-2-thienyl, 3,5-dichloropyridin-4-yl and 3,5-dimethylisoxazol-4-yl.

In another embodiment of the invention there is provided a compound of the formula I selected from:
N-[(3,5-dimethylisoxazol-4-yl)carbonyl]-3-(5-{3-[6-(methylamino)pyridin-2-yl]propyl}thiophen-2-yl)-L-alanine; and
N-[(3,5-dichloropyridin-4-yl)carbonyl]-3-(5-{3-[6-(methylamino)pyridin-2-yl]propyl}thiophen-2-yl)-L-alanine;
   or a pharmaceutically acceptable salt thereof.

In another embodiment of the invention there is provided a compound of the formula I which is N-[(3,5-dimethylisoxazol-4-yl)carbonyl]-3-{5-[3-(5,6,7,8-tetrahydro-1,8-naphthyridin-2-yl)propyl]thiophen-2-yl}-L-alanine, or a pharmaceutically acceptable salt thereof.

In another embodiment of the invention there is provided a compound of the formula I which is N-(3,5-dichloroisonicotinoyl)-3-{5-[3-(5,6,7,8-tetrahydro-1,8-naphthyridin-2-yl)propyl]-thiophen-2-yl}-L-alanine, or a pharmaceutically acceptable salt thereof.

In another embodiment of the invention there is provided a compound of the formula I which is N-(3,5-dichloroisonicotinoyl)-3-{5-[2-(5,6,7,8-tetrahydro-1,8-naphthyridin-2-yl)ethoxy]-2-thienyl}-L-alanine, or a pharmaceutically acceptable salt thereof.

### Synthesis

The compounds of the present invention can be prepared in a number of ways using methods analogous to well known methods of organic synthesis. More specifically, the novel compounds of this invention may be prepared using the reactions and techniques described herein. In the description of the synthetic methods described below, it is to be understood that all proposed reaction conditions, including choice of solvent, reaction atmosphere, reaction temperature, duration of the experiment and workup procedures, are chosen to be the conditions standard for that reaction. It is understood by one skilled in the art of organic synthesis that the functionality present on various portions of the molecule must be compatible with the reagents and reactions proposed. Such restrictions to the substituents, which are not compatible with the reaction conditions, will be apparent to one skilled in the art and alternate methods must then be used.

It will be appreciated that during certain of the following processes certain substituents may require protection to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method as described in the literature or known to the skilled chemist as appropriate for the removal of the protecting group in question, such methods being chosen so as to effect removal of the protecting group with minimum disturbance of groups elsewhere in the molecule.

Thus, if reactants include, for example, groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

Resins may also be used as a protecting group.

The protecting groups may be removed at any convenient stage in the synthesis using conventional techniques well known in the chemical art.

Compounds of the formula I, or pharmaceutically-acceptable salts or prodrugs thereof, may be prepared by any process known to be applicable to the preparation of chemically-related compounds. Such processes, when used to prepare a compound of the formula I, or a pharmaceutically-acceptable salt or prodrug thereof, are provided as a further feature of the invention and are illustrated by the following representative examples. Necessary starting materials may be obtained by standard procedures of organic chemistry (see, for example, Advanced Organic Chemistry (Wiley-Interscience), Jerry March). The preparation of such starting materials is described within the accompanying non-limiting Examples. Alternatively, necessary starting materials are obtainable by analogous procedures to those illustrated which are within the ordinary skill of an organic chemist.

The present invention also provides that compounds of the formula I, or pharmaceutically acceptable salts or prodrugs thereof, can be prepared by a process (a) to (m) as follows (wherein the variables are as defined above unless otherwise stated):

Process (a) for the preparation of those compounds of formula I wherein Z is N(R²⁶), O or S, by reacting a compound of the formula II:

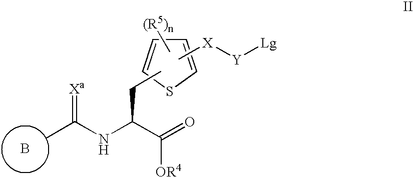

wherein B, R⁴, R⁵, X^{a}, A, Z, Y and n are as hereinbefore defined, except any functional group is protected if necessary, and

Lg is a displaceable group,

with a compound of the formula III:

R⁶-ZH III

wherein R⁶ and Z are as hereinbefore defined, except any functional group is protected if necessary; or

Process (b) for the preparation of those compounds of the formula I wherein X is O, the coupling of a compound of the formula IV:

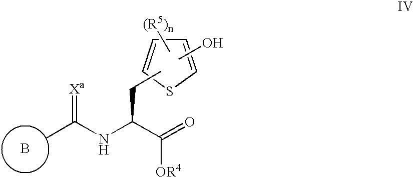

wherein B, R⁴, R⁵, X^{a}, and n are as hereinbefore defined, except any functional group is protected if necessary,

with a compound of the formula V:

R⁶-Z-Y—OH V

wherein R⁶, Y and Z are as hereinbefore defined, except any functional group is protected if necessary; or

Process (c) for the preparation of those compounds of formula I wherein X is O, N(R²⁶) or S by reacting a compound of the formula VI:

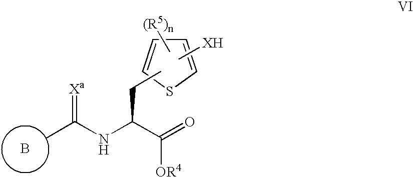

wherein B, R⁴, R⁵, X^{a} and n are as hereinbefore defined, except any functional group is protected if necessary,

with a compound of the formula VII:

R⁶-Z-Y-Lg¹ VII

wherein R⁶, Y and Z are as hereinbefore defined, except any functional group is protected if necessary, and

Lg¹ is a displaceable group; or

Process (d) for the preparation of those compounds of the formula I wherein Z is wherein Z is —C═C—, —C≡C— or the group —Y-Z is alkylene, the reaction of a compound of the formula VIII:

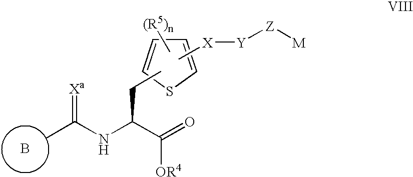

wherein B, R⁴, R⁵, X^{a}, X, Y and n are as hereinbefore defined, except any functional group is protected if necessary,

and M is a suitable displaceable group,

with a compound of the formula R⁶Lg²,

wherein R⁶ is as hereinbefore defined, except any functional group is protected if necessary, and

Lg² is a displaceable group; or

Process (e) for the preparation of those compounds of the formula I wherein X is N(R²⁶)C(O), the coupling of a compound of the formula IX:

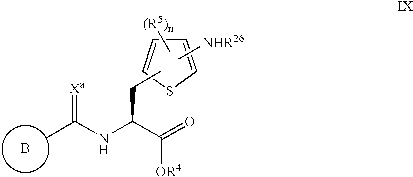

wherein B, R⁴, R⁵, R²⁶, X^{a} and n are as hereinbefore defined, except any functional group is protected if necessary, with a compound of the formula X, or a reactive derivative thereof:

R⁶-Z-Y—C(O)OH X

wherein R⁶, Y and Z are as hereinbefore defined, except any functional group is protected if necessary; or

Process (f) for compounds of formula (I) where X^{a} is oxygen, the coupling of a compound of the formula XI:

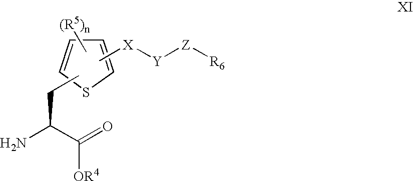

XI

wherein R⁴, R⁵, R⁶, X, Y, Z and n are as hereinbefore defined, except any functional group is protected if necessary,

with a compound of the formula XII, or a reactive derivative thereof:

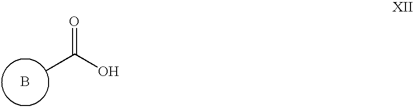

wherein B is as hereinbefore defined, except any functional group is protected if necessary; or

Process (g) for the preparation of those compounds of the formula I wherein X is C(O)N(R²⁶), the coupling of a compound of the formula XIII, or a reactive derivative thereof:

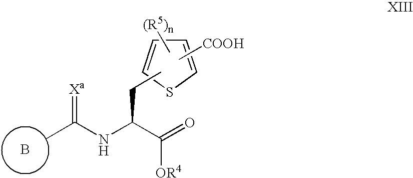

wherein B, R⁴, R⁵, X^{a}, and n are as hereinbefore defined, except any functional group is protected if necessary,

with a compound of the formula XIV:

R⁶-Z-Y—NH(R²⁶) XIV

wherein R⁶, Y, Z and R²⁶ are as hereinbefore defined, except any functional group is protected if necessary; or

Process (h) for the preparation of those compounds of the formula I wherein X is N(R²⁶), O or S, the coupling of a compound of the formula XV:

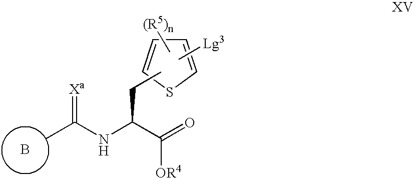

wherein B, R⁴, R⁵, X^{a} and n are as hereinbefore defined, except any functional group is protected if necessary, and

Lg³ is a suitable displaceable group,

with a compound of the formula XVI:

R⁶-Z-Y—X—H XVI

wherein R⁶, X, Y and Z are as hereinbefore defined, except any functional group is protected if necessary; or

Process (i) for the preparation of those compounds of the formula I wherein X is —C═C—, —C≡C— or the group —X—Y is alkylene, the coupling of a compound of the formula XV:

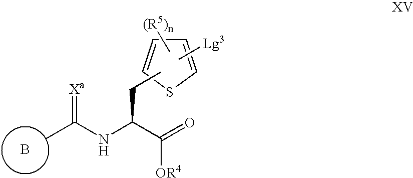

wherein B, R⁴, R⁵, X^{a}, Lg³ and n are as hereinbefore defined, except any functional group is protected if necessary,

with a compound of the formula XVII:

R⁶-Z-Y—X-M XVII

wherein R⁶, Y and Z are as hereinbefore defined, except any functional group is protected if necessary,

and M is a suitable displaceable group; or

Process (j) for the preparation of those compounds of the formula I wherein Z is N(R²⁶), the coupling of a compound of the formula XVIII:

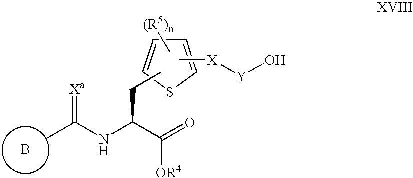

wherein B, R⁴, R⁵, A, X^{a}, X, Y and n are as hereinbefore defined, except any functional group is protected if necessary,

with a compound of the formula XIX:

R⁶—N(R²⁶)H XIX

wherein R⁶ and R²⁶ are as hereinbefore defined, except any functional group is protected if necessary; or

Process (k) for the preparation of those compounds of formula I wherein Z is N(R²⁶), O or S, by reacting a compound of the formula XX:

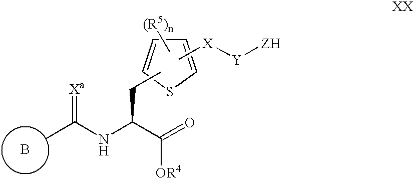

wherein B R⁴, R⁵, X^{a}, X, Y, Z and n are as hereinbefore defined, except any functional group is protected if necessary,

with a compound of the formula XXI:

R⁶-Lg XXI

wherein R⁶ is as hereinbefore defined, except any functional group is protected if necessary, and

Lg is a displaceable group; or

Process (l) for the preparation of those compounds of the formula I wherein the group —X—Y-Z- contains an alkylene chain of at least 3 carbon atoms in length, the hydrogenation of the product of Process (d) or (i) described herein; or

Process (m) for the preparation of those compounds of the formula I where Xa is a sulfur, by reacting a compound of the formula (I) of the formula XXII:

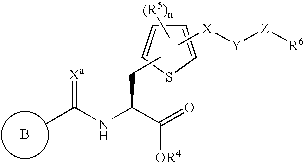

wherein B, R⁴, R⁵, R⁶, X, Y, Z and n are as hereinbefore defined, except any functional group is protected if necessary,

with a thiation reagent such as S₈ or Lawesson reagent (2,4-bis-(4-methoxyphenyl)-1,3-dithia-2,4-diphosphetane 2,4-disulfide);

Process (n) for the preparation of those compounds of the formula I wherein —X—Y-Z- contains a (1-6C)alkoxy or substituted (1-6C)alkoxy group or a (1-6C)alkylamino or substituted (1-6C)alkylamino group, the alkylation, conveniently in the presence of a suitable base as defined hereinbefore, of the corresponding alcohol or amine in which X, Y or Z contains a hydroxy group or a primary or secondary amino group as appropriate; or a reductive amination in which X, Y or Z contains a primary or secondary amino group as appropriate; or
Process (o) when R⁶ is 5,6,7,8-tetrahydro-1,8-naphthyridin-2-yl; the reduction of a compound of the formula XXIV:

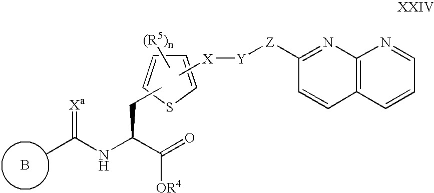

wherein B, R¹, R², R³, R⁴, R⁵, X, Y, Z, n and m are as hereinbefore defined, except any functional group is protected if necessary;

and thereafter, if necessary (in any order):

(i) converting a compound of the formula I into another compound of the formula I;
(ii) removing any protecting groups; and
(iii) forming a pharmaceutically acceptable salt of the compound of formula I.

Suitably in any one of processes (a) to (l), (n) or (o), X^{a} is oxygen and once complete process (m) is conducted where it is desired that X^{a} is S. However, if required thiation can be carried out on any of the intermediates used in the process to ensure that X^{a} is sulphur in the final product.

Specific conditions for the above reactions are as follows.

### Reaction Conditions for Process (a)

A convenient displaceable group Lg is, for example, a halo, alkanesulfonyloxy or arylsulfonyloxy group, for example a chloro, bromo, methanesulfonyloxy, trifluoromethanesulfonyloxy, 4-nitrobenzenesulfonyloxy or toluene-4-sulfonyloxy group.

The reaction is advantageously carried out in the presence of base. A suitable base is, for example, an organic amine base such as, for example, pyridine, 2,6-lutidine, collidine, 4-dimethylaminopyridine, triethylamine, N-methylmorpholine or diazabicyclo[5.4.0]undec-7-ene, or for example, an alkali metal or alkaline earth metal carbonate or hydroxide, for example sodium carbonate, potassium carbonate, cesium carbonate, calcium carbonate, sodium hydroxide or potassium hydroxide. Alternatively such a base is, for example, an alkali metal hydride, for example sodium hydride, an alkali metal or alkaline earth metal amide, for example sodium amide or sodium bis(trimethylsilyl)amide, or a sufficiently basic alkali metal halide, for example cesium fluoride or sodium iodide. The reaction is suitably effected in the presence of an inert solvent or diluent, for example a dipolar aprotic solvent such as N N-dimethylformamide, N,N-dimethylacetamide, N-methylpyrrolidin-2-one or dimethylsulfoxide. The reaction is conveniently effected at a temperature in the range, for example, 10 to 150° C. (or the boiling point of the solvent), suitably in the range 20 to 90° C.

Compounds of the formula II may be prepared using methods well known to those skilled in organic chemistry. Representative methods are illustrated in the Examples described herein.

Compounds of the formula III are commercially available, or they are known in the literature, or they can be prepared by standard processes known in the art.

Reaction Conditions for Process (b)

The coupling reaction is suitably carried out using the Mitsunobu reaction. Suitable Mitsunobu conditions include, for example, reaction in the presence of a suitable tertiary phosphine and a di-alkylazodicarboxylate in an organic solvent such as an ether, for example THF or a halogenated solvent such as methylene chloride. The reaction is suitably carried out in the temperature range −15° C. to 60° C., for example at or near ambient temperature. A suitable tertiary phosphine includes for example tri-n-butylphosphine or particularly tri-phenylphosphine. A suitable di-alkylazodicarboxylate includes for example diethyl azodicarboxylate (DEAD) or di-tert-butyl azodicarboxylate (DTAD) or azodicarbonyldipiperidine (DPAD or ADDP). Details of Mitsunobu reactions are contained in Tet. Letts., 31, 699, (1990); The Mitsunobu Reaction, D. L. Hughes, Organic Reactions, 1992, Vol. 42, 335-656 and Progress in the Mitsunobu Reaction, D. L. Hughes, Organic Preparations and Procedures International, 1996, Vol. 28, 127-164.

Compounds of the formula IV may be prepared using methods well known to those skilled in organic chemistry. Representative methods are illustrated in the Examples described herein.

Compounds of the formula V are commercially available, or they are known in the literature, or they can be prepared by standard processes known in the art.

Reaction Conditions for Process (c)

Suitable leaving groups represented by Lg¹ include those described above for Lg in Process (a), for example halo, such as bromo. The reaction is suitably carried out in the presence of a base, for example a base as hereinbefore described in relation to Process (a) such as an alkali metal or alkaline earth metal carbonate for example potassium carbonate.

The reaction is suitably effected in the presence of an inert solvent or diluent, for example a dipolar aprotic solvent such as N,N-dimethylformamide, N,N-dimethylacetamide, N-methylpyrrolidin-2-one or dimethylsulfoxide. The reaction is conveniently effected at a temperature in the range, for example, 10 to 150° C. (or the boiling point of the solvent), suitably in the range 20 to 90° C.

Compounds of the formula VI may be prepared using methods well known to those skilled in organic chemistry. Representative methods are illustrated in the Examples described herein.

Compounds of the formula VII are commercially available, or they are known in the literature, or they can be prepared by standard processes known in the art.

Reaction Conditions for Process (d)

Suitable coupling reactions are well known to those of ordinary skill in the art of organic chemistry. For example coupling under Heck, Suzuki, Stille, Negishi or when Z is —C≡C—, Sonogashira coupling conditions.

A Heck reaction is suitable for those compounds where Z is —C═C═ and M in formula VIII is H. For a Heck reaction, a suitable displaceable group Lg² is, for example, as hereinbefore defined for Lg, particularly a halo such as, for example, bromo or iodo; and M is H.

Suitable conditions for the Heck reaction are well known such as those described in Syn Lett, 12, 1877 (2005). For example, reaction in the presence of a tertiary base, and a palladium-based catalyst in an inert solvent. The reaction is suitably carried out in the temperature range of 25° C. to 150° C. under thermal or microwave conditions. A suitable tertiary base includes for example triethylamine, N,N-diisopropylethylamine. A suitable palladium catalyst includes palladium(II) acetate in the presence of a phosphine ligand such as tri-phenylphosphine, tri-o-tolylphosphine (Hermman's catalyst), tri-n-butylphosphine. Suitable solvents include N,N-dimethylformamide, tetrahydrofuran, 1,4-dioxane, N,N-dimethylacetamide and 1,2-dimethyoxyethane.

When a Stille coupling is used, Lg² is suitably a halo such as chloro, bromo or iodo, or pseudo halide such as a triflate; and M is a suitable stannane, for example a trialkylstannane such as tributylstannyl, (Bu)₃Sn—. The Stille coupling is carried out in the presence of a suitable palladium catalyst. Suitably the reaction is carried out in a polar solvent such as DMF.

When a Suzuki reaction is used Lg² is suitably a halo such as chloro, bromo or iodo, or pseudo halide such as a triflate; and M is boronic acid or a suitable derivative thereof. For example, M may be a boronic acid ester, potassium trifluoroborate or an organoborane. The coupling reaction is performed in the presence of a palladium catalyst and a suitable base. Suitable bases are as hereinbefore defined.

When a Negishi coupling is used Lg² is suitably a halo such as chloro, bromo or iodo, triflate or acetoxy; and M is an organo zinc group such as a zinc halide, for example ZnI. The reaction is performed in the presence of a suitable palladium or nickel catalyst. The reaction is conveniently performed in the presence of an inert organic solvent such as NMP, THF or DMA.

When Z is —C≡C—, Sonogashira coupling conditions may also be used, wherein Lg² is suitably a halo such as chloro, bromo or iodo or triflate; and M is hydrogen. The reaction is performed in the presence of a suitable palladium catalyst, such as a Pd(0) catalyst or bis triphenylphosphine palladium(II) chloride, and a suitable copper (I) catalyst, such as a copper(I) halide, for example copper iodide. The reaction is suitably performed in the presence of a suitable base, for example a tertiary base such as triethylamine. The reaction is suitably carried out in the temperature range of 25° C. to 150° C. under thermal or microwave conditions. Suitable solvents include N,N-dimethylformamide, N,N-dimethylacetamide and toluene.

Suitable conditions for the Suzuki, Stille, Negishi and Sonogashira are well known and are described in, for example, “Metal-catalysed Cross-Coupling reactions Edited by Armin de Meijere and François Diederich; Wiley-VCH Verlag 2^{nd} Edition 2004.

As will be realised, generally the alternative coupling reactions are also expected to be suitable wherein M is on R⁶ and Lg² is attached to Z.

Compounds of the formula VIII, or corresponding compounds wherein Lg² is attached to Z may be prepared using methods well known to those skilled in organic chemistry. Representative methods are illustrated in the Examples described herein.

Compounds of the formula R⁶Lg² and R⁶-M are commercially available, or they are known in the literature, or they can be prepared by standard processes known in the art.

Reaction Conditions for Process (e)

The coupling reaction may be carried out using standard methods for the coupling of acids and amines. The coupling reaction is conveniently carried out in the presence of a suitable coupling reagent. Standard peptide coupling reagents known in the art can be employed as suitable coupling reagents for example O-(Benzotriazol-1-yl)-N,N,N′,N′-tetramethyluronium tetrafluoroborate (TBTU) or O-(7-Azabenzotriazol-1-yl)-N,N,N′,N′-tetramethyluronium hexafluoro-phosphate (HATU), or for example carbonyldiimidazole, a carbodiimide such as dicyclohexylcarbodiimide or N-ethyl-N′-(3-dimethylaminopropyl)carbodiimide, optionally in the presence of a catalyst such as dimethylaminopyridine, 4-pyrrolidinopyridine or 2-hydroxy-pyridine-N-oxide, optionally in the presence of a base for example triethylamine, N-methylmorpholine, pyridine, or 2,6-di-alkyl-pyridines such as 2,6-lutidine or 2,6-di-tert-butylpyridine. The reaction is conveniently performed in the present of a suitable inert solvent. Suitable solvents include N,N-dimethylacetamide, dichloromethane, benzene, tetrahydrofuran and N,N-dimethylformamide. The coupling reaction is conveniently performed at a temperature in the range of −40 to 40° C.

A “reactive derivative” of the acid of the formula X is a carboxylic acid derivative that will react with the amine of the formula IX to give the corresponding amide. A suitable reactive derivative of a carboxylic acid of the formula X is, for example, an acyl halide, for example an acyl chloride formed by the reaction of the acid and an inorganic acid chloride, for example thionyl chloride; a mixed anhydride, for example an anhydride formed by the reaction of the acid and a chloroformate such as isobutyl chloroformate; an active ester, for example an ester formed by the reaction of the acid and a phenol such as pentafluorophenol in the presence of a suitable coupling agent (such as an carbodiimide), an ester such as pentafluorophenyl trifluoroacetate or an alcohol such as methanol, ethanol, isopropanol, butanol or N-hydroxybenzotriazole; or an acyl azide, for example an azide formed by the reaction of the acid and azide such as diphenylphosphoryl azide; an acyl cyanide, for example a cyanide formed by the reaction of an acid and a cyanide such as diethylphosphoryl cyanide. The reaction of such reactive derivatives of carboxylic acid with amines is well known in the art, for example they may be reacted in the presence of a base, such as those described above, and in a suitable solvent, such as those described above. The reaction may conveniently be performed at a temperature as described above.

Compounds of the formula IX may be prepared using methods well known to those skilled in organic chemistry. Representative methods are illustrated in the Examples described herein.

Compounds of the formula X are commercially available, or they are known in the literature, or they can be prepared by standard processes known in the art.

Reaction Conditions for Process (f)

The coupling is suitably carried out under analogous conditions to those described above in relation to Process (e) for the coupling of acids and amines. Examples of reactive derivatives of the acid of formula XII are as described in relation to Process (e).

Compounds of the formula XII are commercially available, or they are known in the literature, or they can be prepared by standard processes known in the art.

Compounds of the formula XI may be prepared using methods well known to those skilled in organic chemistry. Representative methods are illustrated in the Examples described herein. For example, compounds of the formula XI of the formula XI′:

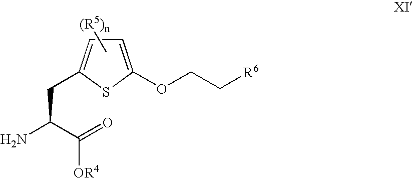

wherein R⁴, R⁵ and n are as hereinbefore defined, except any functional group is protected if necessary, and R⁶ is 5,6,7,8-tetrahydro-1,8-naphthyridin-2-yl, may be prepared using, for example, Reaction Scheme 1:

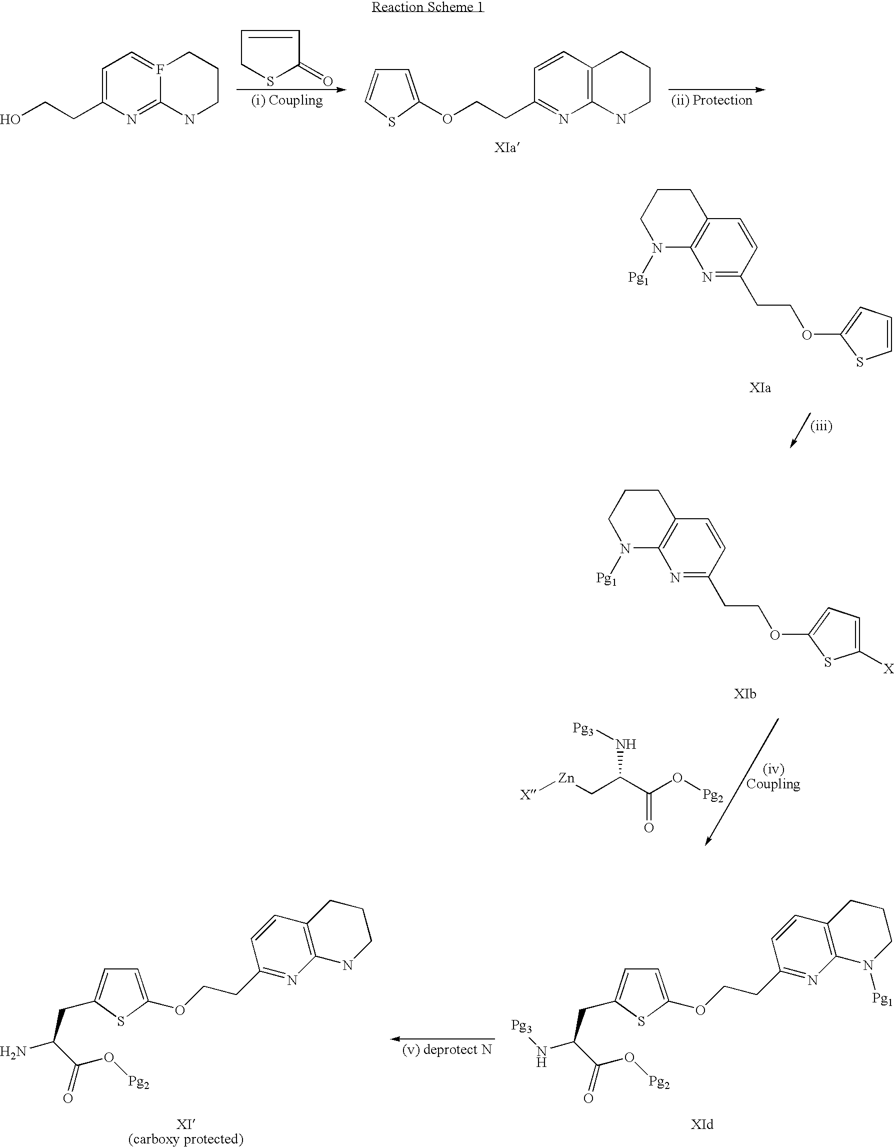

wherein Pg¹ and Pg³ are suitable amino protecting groups, for example tert-butoxycarbonyl (BOC);
Pg² is a suitable carboxy protecting group, for example (1-6C)alkyl such as methyl;
X′ is a suitable displaceable group, for example halo, such as chloro, bromo or iodo, or a sulfonyloxy group such as trifluoromethanesulfonyloxy; and
X″ is halo, particularly chloro, bromo or iodo.

### Notes on Reaction Scheme I

Step (i): The coupling reaction is suitably carried out using analogous conditions to those used in the Mitsunobu reaction. Suitable Mitsunobu conditions include, for example, reaction in the presence of a suitable tertiary phosphine and a di-alkylazodicarboxylate in an organic solvent such as an ether, for example THF or a halogenated solvent such as methylene chloride. The reaction is carried out by pre-mixing the tertiary phosphine and di-alkylazodicarboxylate prior to reaction with the 2-(5,6,7,8-tetrahydro-1,8-naphthyridin-2-yl)ethanol and 2(5H)-thiophenone. Suitably a solution of the 2-(5,6,7,8-tetrahydro-1,8-naphthyridin-2-yl)ethanol and 2(5H)-thiophenone in a suitable solvent such as THF are added to the pre-mix of the tertiary phosphine and di-alkylazodicarboxylate The reaction is suitably carried out in the temperature range −15° C. to 60° C., for example at or near ambient temperature. A suitable tertiary phosphine includes for example tri-n-butylphosphine or particularly tri-phenylphosphine. A suitable di-alkylazodicarboxylate includes for example diethyl azodicarboxylate (DEAD), di-tert-butyl azodicarboxylate (DTAD), di-isopropyl azodicarboxylate or azodicarbonyldipiperidine (DPAD or ADDP).
Step (ii): Protection of the amine by reaction with a suitable protecting agent, for example where Pg¹ is a BOC group, by reaction with di-tert-butyl dicarbonate.
Step (iii): Introduction of displaceable group X′. For example when X′ is halo, such as iodo, by reaction with the appropriate N-halosuccinimide, such as N-iodosuccinimide.
Step (iv): The coupling is suitably performed using the Negishi coupling reaction. Suitable conditions for the Negishi reaction are well known and are described in, for example, “Metal-catalysed Cross-Coupling reactions Edited by Armin de Meijere and François Diederich; Wiley-VCH Verlag 2^{nd} Edition 2004. For example, the coupling reaction may be performed in the presence of a suitable palladium or nickel catalyst in the presence of a suitable ligand. For example tris(dibenzylideneacetone) dipalladium (0.320 g, 0.35 mmol) in the presence of dicyclohexyl-(2′,6′-diisopropoxybiphenyl-2-yl)phosphine. The reaction is conveniently performed in the presence of an inert organic solvent such as NMP, THF, DMA or DMF. The reaction is suitably carried out at elevated temperature, for example 30 to the reflux temperature of the solvent, typically at about 70° C.

The compound of formula XIc is conveniently formed in-situ by reacting a compound of the formula XIc′:

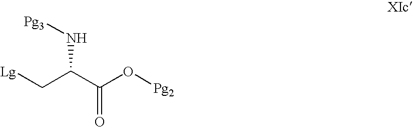

wherein Lg is a suitable displaceable group, for example halo, such as iodo; and Pg² and Pg³ are as hereinbefore defined, for example BOC,

with an appropriate zinc dihalide. Suitable conditions are illustrated in the Examples.

Compounds of the formula XIc′ are known or can be prepared using conventional methods.

Step (v): Deprotection using conventional methods. For example when Pg¹ and Pg³ are BOC groups, by treatment with a suitable acid such as trifluoroacetic acid.

It is expected that analogous methods to those described in Reaction Scheme I would be suitable for the preparation of other compounds of the formula XI wherein the thienyl in formula XI is 2,5-thien-di-yl, X is O and Y, Z, R¹, R⁵, R⁶ and n are as hereinbefore defined, except any functional group is protected if necessary.

Reaction Conditions for Process (g)

The coupling may be carried out under analogous conditions to those described above in relation to Process (e) for the coupling of acids and amines. Suitable reactive derivatives of the compound of the formula XIII are carboxylic acid derivatives such as those described in relation to reactive derivatives of the compound of formula XII described hereinbefore.

Compounds of the formula XIII may be prepared using methods well known to those skilled in organic chemistry.

Compounds of the formula XIV are commercially available, or they are known in the literature, or they can be prepared by standard processes known in the art.

Reaction Conditions for Process (h)

Lg³ is a suitable displaceable group as hereinbefore defined in relation to Lg such as trifluoromethanesulfonyloxy or toluene-4-sulfonyloxy group or particularly halo such as bromo or iodo.

The coupling reaction may be carried out under known conditions for the coupling of aromatic groups, for example using an Ullmann type reaction. Suitable conditions for the Ullmann type reaction include, for example, reaction in the presence of a base, a copper (I)-based catalyst in an inert solvent. The reaction is suitably carried out in the temperature range of 25° C. to 150° C. under thermal or microwave conditions. A suitable base includes for example cesium carbonate. A suitable catalyst includes copper iodide in the presence of a ligand such as L-proline or 1,10-phenanthroline. Suitable solvents include N,N-dimethylformamide, N,N-dimethylacetamide and dimethylsulfoxide.

When X is N(R²⁶), the coupling may also be performed using the Buchwald reaction. Suitable conditions for the Buchwald reaction include, for example, reaction in the presence of a suitable base, a palladium-based catalyst in an inert solvent. The reaction is suitably carried out in the temperature range of 25° C. to 150° C. under thermal or microwave conditions. A suitable base includes for example an alkoxide base such as NaOt-Bu or a carbonate such as cesium carbonate. A suitable palladium catalyst includes bis(dibenzylideneacetone)palladium(0) in the presence of a phosphine ligand such as xantphos. Suitable solvents include N,N-dimethylformamide, N,N-dimethylacetamide, dimethylsulfoxide and toluene.

Alternatively, when X is O Lg³ may be boronic acid or a suitable derivative thereof. For example, Lg³ may is boronic acid. Suitably the coupling is performed in the presence of a copper(II)-based catalyst, optionally in the presence of oxygen.

Compounds of the formula XV may be prepared using methods well known to those skilled in organic chemistry.

Compounds of the formula XVI are commercially available, or they are known in the literature, or they can be prepared by standard processes known in the art.

Reaction Conditions for Process (i)

The coupling reaction may be carried out under Heck, Suzuki, Stille, Negishi or when Z is —C≡C—, Sonogashira coupling conditions as described in relation to Process (d) above. In an alternative reaction the coupling are expected to be suitable for coupling where M is on the thienyl ring in formula XV and Lg³ is on X in the compound of formula XVII.

Compounds of the formula XVII are commercially available, or they are known in the literature, or they can be prepared by standard processes known in the art.

Reaction Conditions for Process (j)

The coupling reaction may be carried out using the Mitsunobu reaction as described in relation to Process (b).

Compounds of the formula XVIII may be prepared using methods well known to those skilled in organic chemistry. Representative methods are illustrated in the Examples described herein.

Compounds of the formula XIX are commercially available, or they are known in the literature, or they can be prepared by standard processes known in the art.

Reaction Conditions for Process (k)

Suitable displaceable groups represented by Lg include those described in relation to Process (a), such as halo, for example chloro. The reaction may be performed under analogous conditions to those described for Process (a), conveniently in the presence of a suitable base such as a carbonate, for example sodium carbonate.

Compounds of the formula XX may be prepared using methods well known to those skilled in organic chemistry. Representative methods are illustrated in the Examples described herein.

Compounds of the formula XXI are commercially available, or they are known in the literature, or they can be prepared by standard processes known in the art.

Reaction Conditions for Process (1)

Hydrogenation conditions are well known in the art, and may include hydrogenation in the presence of a suitable catalyst such as a platinum on carbon or palladium on charcoal.

Reaction Conditions for Process (m)

Suitable conditions for the transformation of an amide into a thioamide include, for example, reaction in the presence of Lawesson reagent or S₈ in an inert solvent. The reaction is suitably carried out in the temperature range of 25° C. to 150° C. under thermal or microwave conditions. Suitable solvents include for instance toluene. Compounds of the formula XXII may be prepared using any of the processes (a) to (1) described above.

Reaction Conditions for Process (n)

A suitable alkylating agent is, for example, any agent known in the art for the alkylation of hydroxy to alkoxy or substituted alkoxy, or for the alkylation of amino to alkylamino or substituted alkylamino, for example an alkyl or substituted alkyl halide, for example a (1-6C)alkyl chloride, bromide or iodide or a substituted (1-6C)alkyl chloride, bromide or iodide, conveniently in the presence of a suitable base as defined hereinbefore, in a suitable inert solvent or diluent as defined hereinbefore and at a temperature in the range, for example, 10 to 140° C., conveniently at or near ambient temperature. Conveniently for the production of those compounds of the formula I wherein —X—Y-Z- contains a (1-6C)alkylamino or substituted (1-6C)alkylamino group, a reductive amination reaction may be employed. For example, for the production of those compounds of the Formula I wherein X, Y or Z contains a N-alkyl group, the corresponding compound containing a N—H group may be reacted with formaldehyde (to give an N-methyl group), an appropriate aldehyde (to give an N-alkyl group) or an appropriate ketone (to give a N-substituted alkyl group) in the presence of a suitable reducing agent. A suitable reducing agent is, for example, a hydride reducing agent, for example an alkali metal aluminium hydride such as lithium aluminium hydride or, preferably, an alkali metal borohydride such as sodium borohydride, sodium cyanoborohydride, sodium triethylborohydride, sodium trimethoxyborohydride and sodium triacetoxyborohydride. The reaction is conveniently performed in a suitable inert solvent or diluent, for example tetrahydrofuran and diethyl ether for the more powerful reducing agents such as lithium aluminium hydride, and, for example, methylene chloride or a protic solvent such as methanol and ethanol for the less powerful reducing agents such as sodium triacetoxyborohydride and sodium cyanoborohydride. The reaction is performed at a temperature in the range, for example, 10 to 80° C., conveniently at or near ambient temperature. Suitable reductive amination conditions are well known, for example as described in Abdel-Magid, Ahmed F.; Mehrman, Steven J. A Review on the Use of Sodium Triacetoxyborohydride in the Reductive Amination of Ketones and Aldehydes. Organic Process Research & Development (2006), 10(5), 971-1031; or Baxter, Ellen W.; Reitz, Allen B. Reductive aminations of carbonyl compounds with borohydride and borane reducing agents. Organic Reactions (New York) (2002), 591-714. CODEN: ORREAW ISSN:0078-6179. CAN 138:169565 AN 2002:450507 CAPLUS.

Reaction Conditions for Process (o)

The reduction may be performed using a suitable reducing agent, for example by hydrogenation in the presence of a suitable catalyst such as a platinum on carbon or palladium on charcoal catalyst as illustrated in the examples herein.

The compound of formula XXIV may conveniently be prepared by reacting the methyl ketone of formula XXIVa with the compound of formula XXIVb

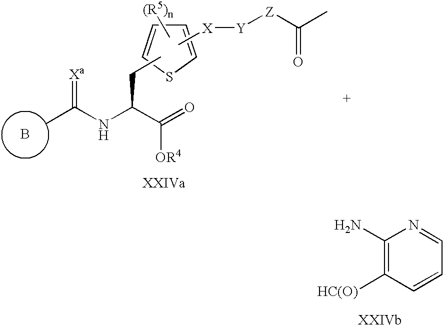

wherein B, R⁴, R⁵, X, Y, Z and n m are as hereinbefore defined, except any functional group is protected if necessary. The reaction is performed in the presence of a suitable base such as L-proline, or other an alkali metal hydroxide. Conveniently the reaction is performed in the presence of a suitable organic solvent such as an alcohol for example methanol, ethanol or isopropyl alcohol or an ether such as THF. Suitable reaction conditions and the preparation of the compound of formulae XXIVa are illustrated in the examples.

For example compounds of the formula XXIVa wherein X—Y-Z- is —(CH₂)₃— may be prepared as illustrated in the reaction scheme below:

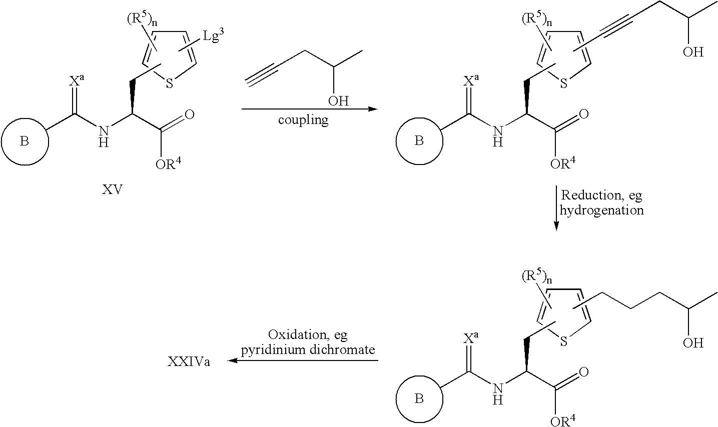

wherein B, X^{a}, R⁴, R⁵, Lg³ and n are as hereinbefore defined, except any functional group is protected if necessary.

Compounds of the formula I may also be obtained by modifying a substituent in or introducing a substituent into another compound of formula I or a pharmaceutically acceptable salt or prodrug thereof. Suitable chemical transformations are well known to those in the art of organic chemistry. For example, when R⁴ is (1-6C)alkyl in a compound of formula I, the alkyl group may be replaced by hydrogen by hydrolysis of the compound of formula I to give another compound of formula I in which R⁴ is hydrogen. Suitably the hydrolysis is carried out in the presence of a suitable base such as lithium hydroxide. Further representative transformations include the reduction of a —C≡C— or —C═C— group in X, Y or Z to a —CH₂CH₂—. Suitable reducing conditions include for example hydrogenation in the presence of a suitable catalyst such as a platinum on carbon or palladium on charcoal. During these transformations, functional groups may be protected as required, and the protecting groups removed subsequently.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogeno group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulfinyl or alkylsulfonyl; and the alkylation of an amino group by reductive amination. For example when R⁴ is other than hydrogen, esters of the compound of formula I may be prepared by reacting the compound of formula I wherein R⁴ is H with an alcohol R⁴OH using standard methods such as activation of the carboxylic acid with a carbodiimide followed by reaction with the alcohol or coupling under Mitsunobu conditions.

When a pharmaceutically acceptable salt of a compound of the formula I is required, for example an acid or base addition salt, it may be obtained by, for example, reaction of the compound of formula I with a suitable acid or base using a conventional procedure. Methods for the preparation of pharmaceutically acceptable salts are well known in the art. For example, following reaction of a compound of the formula I with an acid or base the required salt may be precipitated from solution by supersaturating the solution containing the compound of the formula I. Super saturation may be achieved using well-known techniques, for example by cooling the solution, by removing solvent by evaporation or by the addition of a suitable anti-solvent to precipitate the salt.

To facilitate isolation of a compound of the formula I during its preparation, the compound may be prepared in the form of a salt that is not a pharmaceutically acceptable salt. The resulting salt can then be modified by conventional techniques to give a pharmaceutically acceptable salt of the compound. Such salt modification techniques are well known and include, for example ion exchange techniques or re-precipitation of the compound from solution in the presence of a pharmaceutically acceptable counter ion as described above, for example by re-precipitation in the presence of a suitable pharmaceutically acceptable acid to give the required pharmaceutically acceptable acid addition salt of a compound of the formula I.

Stereoisomers of compounds of formula I may be separated using conventional techniques, e.g. chromatography or fractional crystallisation. The enantiomers may be isolated by separation of a racemate for example by fractional crystallisation, resolution or HPLC. The diastereoisomers may be isolated by separation by virtue of the different physical properties of the diastereoisomers, for example, by fractional crystallisation, HPLC or flash chromatography. Alternatively particular stereoisomers may be made by chiral synthesis from chiral starting materials under conditions which will not cause racemisation or epimerisation, or by derivatisation, with a chiral reagent. When a specific stereoisomer is isolated it is suitably isolated substantially free from other stereoisomers, for example containing less than 20%, particularly less than 10% and more particularly less than 5% by weight of other stereoisomers.

In the synthesis section above and hereafter, the expression “inert solvent” refers to a solvent which does not react with the starting materials, reagents, intermediates or products in a manner which adversely affects the yield of the desired product.

Persons skilled in the art will appreciate that, in order to obtain compounds of the invention in an alternative and in some occasions, more convenient manner, the individual process steps mentioned hereinbefore may be performed in different order, and/or the individual reactions may be performed at different stage in the overall route (i.e. chemical transformations may be performed upon different intermediates to those associated hereinbefore with a particular reaction).

Certain of the intermediates used in the above described processes for the preparation of compounds of the formula I form a further aspect of the invention.

### Biological Activity

The following assays can be used to measure the effects of the compounds of the present invention as a5b1 integrin inhibitors.

### (a) In Vitro Binding Assay

The assay determined the ability of compounds to inhibit binding of α5β1 integrin to a cognate ligand, a fragment of human fibronectin. The assay used Origen technology (IGEN International) to measure the compound activity. Briefly, α5β1 integrin was coated onto epoxy-paramagnetic beads (Dynal Biotech UK, Bromborough, Wirral, CH62 3QL, UK, Catalogue No 140.11) and biotinylated-fibronectin ligand was coupled to strepatividin labeled Tag-NHS-Ester (BioVeris Corporation, Witney, Oxfordshire, OX28 4GE, UK, Catalogue No 110034). The ruthenium-labeled tag emits an electrochemiluminescence signal upon stimulation which is detected by the Origen reader. Thus, interaction of integrin and ligand causes association of bead and tag, and the resulting electrochemiluminescence signal reflects the level of integrin interaction with fibronectin.

20 μg of recombinant human α5β1 was coated onto 4×10(7) epoxy-paramagnetic beads according to manufacturers instructions at 4° C. for 24 hours. Integrin coating and corresponding activity was subsequently measured for each batch but typically, 25 ng of coated protein was used per assay well.

A DNA fragment encoding the domains 9-10 (amino-acids 1325-1509) of human fibronectin (Swiss-Prot Accession No. P02751) was isolated from cDNA libraries using standard molecular biology and PCR cloning techniques. The cDNA fragment was sub-cloned into a pT73.3 expression vector containing a GST-epitope tag (developed at AstraZeneca; Bagnall et al., Protein Expression and Purification, 2003, 27: 1-11). Following expression in E. coli, the expressed protein, termed Fn9-10, was purified using the GST-tag using standard purification techniques. The recombinant Fn9-10 was subsequently biotinylated using a EZ-link Sulfo-NHS-LC-Biotinylation kit (Perbio Science UK Ltd., Cramlington, Northumberland, NE231WA, UK, Catalogue No. 21335) and made to give a final concentration of approximately 1 mg/mil. Tag-NHS-Ester was labeled with streptavidin by incubation at room temperature following manufacturers instructions and buffer-exchanged into PBS to give a concentration of 0.5 mg/ml. Immediately prior to the assay, biotinylated-Fn9-10 and Streptavidin-labeled Tag were diluted in Assay Buffer to give a final concentrations of 0.6 ug/ml and 1.5 ug/ml respectively. The Fn9-10 and Tag solutions were then mixed together in equal volumes and incubated on ice for at least 30 minutes prior to the assay.

Test compounds were prepared as 10 mM stock solutions in DMSO (Sigma-Aldrich Company Ltd, Gillingham, Dorset SP8 4XT Catalogue No. 154938) and serially diluted with 4% DMSO to give a range of test concentrations at ×4 required final concentration. Aliquots (20 μl) of each compound dilution were placed into each well of a 384-well round bottomed polypropylene plate (Matrix Technologies, Wilmslow, Cheshire, SK9 3LP, Catalogue No. 4340 384). Each plate also contained control wells: maximum signal was created using wells containing 20 μl of 4% DMSO, and minimum signal corresponding to no binding was created using wells containing 20 μl of 80 mM EDTA (Sigma Catalogue No. E7889).

For the assay, 25 ng (20 ul) of a5b1-bead suspension and 401 of the Fn9-10/Tag pre-incubated solution were added to each well containing 20 μl of compound or control solution. Assay plates were then incubated at room temperature for a minimum of 6 hours before being analysed on an Origen plate reader. The minimum value was subtracted from all values, and the signal was plotted against compound concentration to generate IC₅₀ data.

### (b) In Vitro Cell Adhesion Assay

The assay determined the ability of compounds to inhibit the α5β1 integrin mediated adhesion of K562 cells to the ligand, a fragment of human fibronectin. The human K562 erythroleukaemia cell line (LGC Promochem, Teddington, Middlesex, UK, Catalogue No. CCL-243) was routinely maintained in RPMI 1640 medium (Sigma-Aldrich Company Ltd, Gillingham, Dorset SP8 4XT, Catalogue No. R0883) containing 10% heat-inactivated foetal calf serum (PAA lab GmbH, Pasching, Austria Catalogue No. PAA-A15-043) and 1% glutamax-1 (Invitrogen Ltd. Paisley, UK Catalogue No. 35050-038) at 37° C. with 5% CO₂ at densities between 1×10⁵ and 1×10⁶ cells/ml.

A DNA fragment encoding the domains 9-10 (amino-acids 1325-1509) of human fibronectin (Swiss-Prot Accession No. P02751) was isolated from cDNA libraries using standard molecular biology and PCR cloning techniques. The cDNA fragment was sub-cloned into a pT7#3.3 expression vector containing a GST-epitope tag (developed at AstraZeneca; Bagnall et al., Protein Expression and Purification, 2003, 27: 1-11), and the fragment termed Fn9-10. Following expression in E. coli, the expressed protein was purified using the GST-tag using standard purification techniques.

For adhesion assay, a 96-well flat bottomed plate (Greiner Bio one ltd., Gloucester GL10 3SX Catalogue No. 655101) was coated overnight at 4° C. with 100 μl of 20 μg/ml Fn9-10 ligand in Dulbecco's PBS (Gibco#14190-94). The plate was then washed twice with 200 μl of PBS and blocked with 100 μl of 3% BSA (SigmaA7888) in PBS for 1 hour at 37° C. The plates were then washed again 3 times with 200 μl of PBS and left empty.

Test compounds were prepared as 10 mM stock solutions in DMSO (Sigma-Aldrich Company Ltd, Gillingham, Dorset SP8 4XT Catalogue No. 154938) and serially diluted with HBSS (Hanks Buffered Salt solution (Gibco Catalogue No. 14170-088)/2% DMSO to give a range of test concentrations at twice required final concentration. Aliquots (50 μl) of each compound dilution were placed into each well of the Fn9-10 coated plates. Each plate also contained control wells: maximum adhesion signal was created using wells containing 50 μl HBSS/2% DMSO, and minimum signal corresponding to no adhesion was created using wells containing 50 μl HBSS/2% DMSO/20 mM EDTA (Sigma Catalogue No. E7889).

The K562 cells were cultured to 1×10⁶ cells/ml, and each culture suspension pooled. Cells were centrifuged at 1200 rpm for 2 mins, and the pellets washed with HBSS followed by HBSS/50 mM HEPES (Sigma Catalogue No. H0887). Cell pellets were pooled and re-suspended in HBSS/0.4 mM manganese chloride/50 mM HEPES (MnCl; Sigma Catalogue No. M1787) to give a final concentration of 4×10⁶ cells/ml.

The assay was initiated by the addition of 50 μl of cell suspension into each coated well (200,000 cells/well), thus resulting in final desired compound concentration and a final MnCl concentration of 0.2 mM. The plates were incubated for 45 minutes at 37° C. 5% CO₂. After this time, the solution was flicked off as waste, and the remaining cell layer carefully washed twice with 200 μl of PBS, and then fixed with 200 μl of 100% ethanol for 30 minutes.

After fixation, the ethanol was flicked off to waste and 100 μl of 0.1% Crystal violet stain was added to each well, and incubated at ambient temperature for 15 minutes. Excess stain was removed by rinsing 3 times under cold slow running water. The plates were blotted over tissue then solubilised by adding 50 μl of 1% Triton X100 (Sigma Catalogue No. T9284) and shaking at 500 rpm for 30 mins on plate shaker. Finally, 100 μl of deionised water was added to each well and the absorbance was determined at 590 nM on a spectrophotometer. The minimum value was subtracted from all values, and the absorbance signal was plotted against compound concentration to generate IC₅₀ data.

Although the pharmacological properties of the compounds of the formula I vary with structural change as expected, compounds of the formula I, were found to be active in the above screens. In general activity possessed by compounds of the formula I, may be demonstrated at the following concentrations or doses in one or more of the above tests (a) and (b):
Test (a):—IC₅₀ in the range, for example, 0.1 nM to 10 μM (preferred compounds have an IC₅₀ of less than 1 μM);
Test (b):—IC₅₀ in the range, for example, 1 nM to 20 μM (preferred compounds have an IC₅₀ of less than 5 μM).

By way of example, activity for the following compounds was observed:

| | Binding Assay (a) | Adhesion Assay (b) |
|---|---|---|
| Example | (μM) | (μM) |
| 1.1 | 0.004 | 0.027 |
| | (n = 3) | (n = 2) |
| 1.2 | 0.001 | 0.008 |
| | (n = 2) | (n = 2) |
| 2 | <0.001 | 0.012 |
| | (n = 2) | (n = 3) |
| 3 | 0.0003 | <0.004 |
| | (n = 3) | (n = 3, 0.005, <0.003, |
| | | <0.003) |
| 4 | <0.001 | <0.003 |
| | (n = 2) | (n = 1) |

In the table n indicates the number of tests carried out on each compound and the IC₅₀ values shown represent the geometric mean of the measured IC₅₀ values for each compound.

### Pharmaceutical Compositions

According to a further aspect of the invention there is provided a pharmaceutical composition which comprises a compound of the formula I, or a pharmaceutically acceptable salt or prodrug thereof, as defined hereinbefore in association with a pharmaceutically acceptable diluent or cammer.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy of infection is an amount sufficient to symptomatically relieve in a warm-blooded animal, particularly a human the symptoms of infection, to slow the progression of infection, or to reduce in patients with symptoms of infection the risk of getting worse.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the formula I will naturally vary according to the nature and severity of the conditions, the age and sex of the animal or patient and the route of administration, according to well known principles of medicine.

In using a compound of the formula I for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration may also be suitable, particularly in tablet form. Typically, unit dosage forms will contain about 0.5 mg to 0.5 g of a compound of this invention.

The compounds of the present invention are expected to possess, amongst others, anti-angiogenic properties such as anti-cancer properties that are believed to arise from the inhibition of a5b1 function, particularly the compounds according to the invention are thought to be a5b1 antagonists. Furthermore, the compounds according to the present invention may possess substantially better potency against the a5b1 integrin, than against other integrins such as αvβ3, αiibβ3 or α4β1. Such compounds possess sufficient potency against the a5b1 integrin that they may be used in an amount sufficient to inhibit a5b1 function whilst demonstrating little, or significantly lower, activity against other integrins, such as those mentioned above. Such compounds are likely to be useful as selective a5b1 antagonists and are likely to be useful for the effective treatment of, for example a5b1 driven tumours.

Accordingly, the compounds of the present invention are expected to be useful in the treatment of diseases or medical conditions mediated alone or in part by a5b1 integrin, i.e. the compounds may be used to produce an a5b1 antagonistic effect in a warm-blooded animal in need of such treatment. Thus the compounds of the present invention provide a method for the treatment of malignant cells characterised by inhibition of a5b1 function. Particularly the compounds of the invention may be used to produce anti-angiogenic and/or an anti-proliferative and/or anti-invasive effect mediated alone or in part by the inhibition of a5b1 function. Particularly, the compounds of the present invention are expected to be useful in the prevention or treatment of those tumours that are sensitive to inhibition of a5b1 function that are involved in for example, angiogenesis, proliferation and the signal transduction steps which drive proliferation, invasion and particularly angiogenesis of these tumour cells. Accordingly the compounds of the present invention may be useful in the treatment of hyperproliferative disorders, including psoriasis, benign prostatic hyperplasia (BPH), atherosclerosis and restenosis and/or cancer by providing an anti-proliferative effect, particularly in the treatment of a5b1 sensitive cancers. Such benign or malignant tumours may affect any tissue and include non-solid tumours such as leukaemia, multiple myeloma or lymphoma, and also solid tumours, for example bile duct, bone, bladder, brain/CNS, breast, colorectal, endometrial, gastric, head and neck, hepatic, lung, neuronal, oesophageal, ovarian, pancreatic, prostate, renal, skin, testicular, thyroid, uterine and vulval cancers. The compounds of the invention are expected to be useful in the treatment of pathogenic angiogenesis (pathological angiogenesis), for example in the treatment of cancers as hereinbefore described and other diseases in which inappropriate, or pathogenic angiogenesis occurs. By inappropriate, pathogenic or pathological angiogenesis is meant undesirable angiogenesis that results in an undesirable medical condition or disease such as age-related macular degeneration (AMD) or cancers involving a solid tumour. The compounds of the invention may also be useful in the treatment or prophylaxis of other conditions in which a5b1 is implicated, for example thrombosis, cardiac infarction, coronary heart diseases, arteriosclerosis, tumours, osteoporosis, inflammations such as psoriasis, gingivitis, osteoarthritis, rheumatoid arthritis, irritable bowel syndrome, ulcerative colitis or Crohn's disease, or infections.

In another aspect of the present invention there is provided a compound of formula I, or a pharmaceutically acceptable salt or prodrug thereof, as defined hereinbefore for use as a medicament.

In another embodiment the present invention provides the use of a compound of formula I or a pharmaceutically acceptable salt or prodrug thereof in the preparation of a medicament.

In another embodiment the present invention provides a compound of formula I or a pharmaceutically acceptable salt or prodrug thereof for use in the treatment or prophylaxis of a cancer, for example a cancer involving a solid tumour.

In another embodiment the present invention provides a compound of formula I or a pharmaceutically acceptable salt or prodrug thereof for use in the treatment or prophylaxis of neoplastic disease such as carcinoma of the breast, ovary, lung (including small cell lung cancer, non-small cell lung cancer and bronchioalveolar cancer), colon, rectum, prostate, bile duct, bone, bladder, head and neck, kidney, liver, gastrointestinal tissue, oesophagus, pancreas, skin, testes, thyroid, uterus, cervix, vulva or other tissues, as well as leukemias and lymphomas including CLL and CML, tumors of the central and peripheral nervous system, and other tumor types such as melanoma, multiple myeloma, fibrosarcoma and osteosarcoma, and malignant brain tumors.

In still another embodiment the present invention provides a compound of formula I or a pharmaceutically acceptable salt or prodrug thereof for use in the treatment or prophylaxis of pathologically angiogenic diseases, thrombosis, cardiac infarction, coronary heart diseases, arteriosclerosis, tumours, osteoporosis, inflammations or infections.

In another embodiment the present invention provides a compound of formula I or a pharmaceutically acceptable salt or prodrug thereof for use in the inhibition of a5b1 function.

In another embodiment the present invention provides a compound of formula I or a pharmaceutically acceptable salt or prodrug thereof for use as an antiangiogenic agent in the treatment of a solid tumour.

In another embodiment the present invention provides the use of a compound of formula I or a pharmaceutically acceptable salt or prodrug thereof in the preparation of a medicament for the treatment or prophylaxis of a cancer, for example a cancer involving a solid tumour.

In another embodiment the present invention provides the use of a compound of formula I or a pharmaceutically acceptable salt or prodrug thereof in the preparation of a medicament for the treatment or prophylaxis of neoplastic disease such as carcinoma of the breast, ovary, lung (including small cell lung cancer, non-small cell lung cancer and bronchioalveolar cancer), colon, rectum, prostate, bile duct, bone, bladder, head and neck, kidney, liver, gastrointestinal tissue, oesophagus, pancreas, skin, testes, thyroid, uterus, cervix, vulva or other tissues, as well as leukemias and lymphomas including CLL and CML, tumors of the central and peripheral nervous system, and other tumor types such as melanoma, multiple myeloma, fibrosarcoma and osteosarcoma, and malignant brain tumors.

In still another embodiment the present invention provides the use of a compound of formula I or a pharmaceutically acceptable salt or prodrug thereof in the preparation of a medicament for the treatment or prophylaxis of pathologically angiogenic diseases, thrombosis, cardiac infarction, coronary heart diseases, arteriosclerosis, tumours, osteoporosis, inflammations or infections.

In another embodiment the present invention provides the use of a compound of formula I or a pharmaceutically acceptable salt or prodrug thereof in the preparation of a medicament for use in the inhibition of a5b1 function.

In another embodiment the present invention provides the use of a compound of formula I or a pharmaceutically acceptable salt or prodrug thereof in the manufacture of a medicament for use as an antiangiogenic agent in the treatment of a solid tumour.

In a further aspect of the invention there is provided a pharmaceutical composition which comprises a compound of the formula I, or a pharmaceutically acceptable salt or prodrug thereof, as defined herein before in association with a pharmaceutically-acceptable diluent or carrier for use in the production of an a5b1 antagonistic effect in a warm-blooded animal such as man.

In a further aspect of the invention there is provided a pharmaceutical composition which comprises a compound of the formula I, or a pharmaceutically acceptable salt or prodrug thereof, as defined herein before in association with a pharmaceutically acceptable diluent or carrier for use in the production of an anti-cancer effect in a warm-blooded animal such as man.

In a further aspect of the invention there is provided a pharmaceutical composition which comprises a compound of the formula I, or a pharmaceutically acceptable salt or prodrug thereof, as defined herein before in association with a pharmaceutically acceptable diluent or carrier for use as an antiangiogenic agent in the treatment of a solid tumour.

In a further aspect of the invention there is provided a pharmaceutical composition which comprises a compound of the formula I, or a pharmaceutically acceptable salt or prodrug thereof, as defined herein before in association with a pharmaceutically-acceptable diluent or carrier for use in the treatment or prophylaxis of pathologically angiogenic diseases, thrombosis, cardiac infarction, coronary heart diseases, arteriosclerosis, tumours, osteoporosis, inflammations or infections.

In another embodiment the present invention provides a method of inhibiting pathological angiogenesis in a human or animal comprising administering to said human or animal in need of said inhibiting a therapeutically effective amount of a compound of formula I or a pharmaceutically acceptable salt or prodrug thereof.

In a further embodiment the present invention provides a method of inhibiting a5b1 function comprising administering to an animal or human in need of said inhibiting a therapeutically effective amount of a compound of formula I, or a pharmaceutically acceptable salt or prodrug thereof.

In a further embodiment the present invention provides a method of prophylaxis or treatment of a disease mediated in part or alone by a5b1 comprising administering to an animal or human in need of said prophylaxis or treatment a therapeutically effective amount of a compound of formula I, or a pharmaceutically acceptable salt or prodrug thereof.

In another embodiment the present invention provides a method of treatment of a human or animal suffering from cancer comprising administering to said human or animal a therapeutically effective amount of a compound of formula I, or a pharmaceutically acceptable salt or prodrug thereof.

In further embodiment the present invention provides a method of prophylaxis or treatment of cancer comprising administering to a human or animal in need of such prophylaxis or treatment a therapeutically effective amount of a compound of formula I, or a pharmaceutically acceptable salt or prodrug thereof.

In another embodiment the present invention provides a method of prophylaxis or treatment of a human or animal suffering from a neoplastic disease such as carcinoma of the breast, ovary, lung (including small cell lung cancer, non-small cell lung cancer and bronchioalveolar cancer), colon, rectum, prostate, bile duct, bone, bladder, head and neck, kidney, liver, gastrointestinal tissue, oesophagus, pancreas, skin, testes, thyroid, uterus, cervix, vulva or other tissues, as well as leukemias and lymphomas including CLL and CML, tumours of the central and peripheral nervous system, and other tumour types such as melanoma, multiple myeloma, fibrosarcoma and osteosarcoma, and malignant brain tumours, comprising administering to said human or animal a therapeutically effective amount of a compound of formula I, or a pharmaceutically acceptable salt or prodrug thereof.

In another embodiment the present invention provides a method of prophylaxis or treatment of a pathologically angiogenic disease, thrombosis, cardiac infarction, coronary heart diseases, arteriosclerosis, tumours, osteoporosis, inflammation or infection in a human or animal in need of such prophylaxis or treatment comprising administering to said human or animal a therapeutically effective amount of a compound of formula I or a pharmaceutically acceptable salt or prodrug thereof.

### Combination Therapies

The anti-cancer treatment defined hereinbefore may be applied as a sole therapy or may involve, in addition to the compound of the invention, conventional surgery or radiotherapy or chemotherapy. Such chemotherapy may include one or more of the following categories of anti-tumour agents:—

(i) other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) anti-invasion agents [for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341), N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661) and bosutinib (SKI-606), and metalloproteinase inhibitors like marimastat, inhibitors of urokinase plasminogen activator receptor function or antibodies to Heparanase];
(iv) inhibitors of growth factor function: for example such inhibitors include growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stem et al. Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib); inhibitors of the hepatocyte growth factor family; inhibitors of the insulin growth factor family; inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib (BAY 43-9006), tipifamib (R115777) and lonafamib (SCH66336)), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;
(v) antiangiogenic agents such as those which inhibit the effects of vascular endothelial growth factor, [for example the anti-vascular endothelial cell growth factor antibody bevacizumab (Avastin™) and for example, a VEGF receptor tyrosine kinase inhibitor such as vandetanib (ZD6474), vatalanib (PTK787), sunitinib (SU11248), axitinib (AG-013736), pazopanib (GW 786034) and 4-(4-fluoro-2-methylindol-5-yloxy)-6-methoxy-7-(3-pyrrolidin-1-ylpropoxy)quinazoline (AZD2171; Example 240 within WO 00/47212), compounds such as those disclosed in International Patent Applications WO97/22596, WO 97/30035, WO 97/32856 and WO 98/13354 and compounds that work by other mechanisms (for example linomide, inhibitors of integrin αᵥβ₃ function and angiostatin)];
(vi) vascular damaging agents such as Combretastatin A4 and compounds disclosed in International Patent Applications WO 99/02166, WO 00/40529, WO 00/41669, WO 01/92224, WO 02/04434 and WO 02/08213;
(vii) an endothelin receptor antagonist, for example zibotentan (ZD4054) or atrasentan;
(viii) antisense therapies, for example those which are directed to the targets listed above, such as ISIS 2503, an anti-ras antisense;
(ix) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(x) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

Accordingly a further aspect of the invention provides a method of treatment of a cancer comprising administering to a human or animal in need of such treatment a therapeutically effective amount of:

(a) a compound of formula I, or a pharmaceutically acceptable salt or prodrug thereof, as defined hereinbefore; and

(b) an additional chemotherapeutic agent.

Suitable additional chemotherapeutic agents are as hereinbefore defined in relation to combination therapies, for example one or more agents selected from selected from:
(i) an antiangiogenic agent;
(ii) a cytostatic agent;
(iii) an antiproliferative agent
(iv) an antineoplastic agent;
(v) an anti-invasion agent;
(vi) an inhibitor of growth factor function; and
(vii) a vascular damaging agent.

The compounds according to the present invention may also be used together with one or more other anticancer therapies, for example in conjunction with one or more of an anti-cancer therapy selected from an antisense therapy, a gene therapy and an immunotherapy.

According to this aspect of the invention there is provided a combination suitable for use in the treatment of a cancer (for example a cancer involving a solid tumour) comprising a compound of formula I as defined hereinbefore, or a pharmaceutically acceptable salt or prodrug thereof and any one of the anti-tumour agents listed under (i)-(ix) above.

In a further aspect of the invention there is provided a compound of formula I, or a pharmaceutically acceptable salt or prodrug thereof in combination with an anti-tumour agent selected from one listed under (i)-(ix) herein above.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

According to a further aspect of the invention there is provided a pharmaceutical composition which comprises a compound of formula I, or a pharmaceutically acceptable salt or prodrug thereof in combination with an anti-tumour agent selected from one listed under (i)-(ix) herein above, in association with a pharmaceutically acceptable diluent or carrier.

According to a further aspect of the invention there is provided a pharmaceutical composition which comprises a compound of formula I, or a pharmaceutically acceptable salt or prodrug thereof in combination with an anti-tumour agent selected from one listed under (i)-(ix) herein above, in association with a pharmaceutically acceptable diluent or carrier for use in the production of an a5b1 antagonistic effect in a warm-blooded animal such as man. According to a further aspect of the invention there is provided a pharmaceutical composition which comprises a compound of formula I, or a pharmaceutically acceptable salt or prodrug thereof in combination with an anti-tumour agent selected from one listed under (i)-(ix) herein above, in association with a pharmaceutically acceptable diluent or carrier for use as an antiangiogenic agent in the treatment of a solid tumour.

According to a further aspect of the invention there is provided a pharmaceutical composition which comprises a compound of formula I, or a pharmaceutically acceptable salt or prodrug thereof in combination with an anti-tumour agent selected from one listed under (i)-(ix) herein above, in association with a pharmaceutically acceptable diluent or carrier for use in the treatment or prophylaxis of neoplastic disease such as carcinoma of the breast, ovary, lung (including small cell lung cancer, non-small cell lung cancer and bronchioalveolar cancer), colon, rectum, prostate, bile duct, bone, bladder, head and neck, kidney, liver, gastrointestinal tissue, oesophagus, pancreas, skin, testes, thyroid, uterus, cervix, vulva or other tissues, as well as leukemias and lymphomas including CLL and CML, tumors of the central and peripheral nervous system, and other tumor types such as melanoma, multiple myeloma, fibrosarcoma and osteosarcoma, and malignant brain tumors.

According to another feature of the invention there is provided the use of a compound of the formula I, or a pharmaceutically acceptable salt or prodrug thereof in combination with an anti-tumour agent selected from one listed under (i)-(ix) herein above, in the manufacture of a medicament for use in the treatment of a cancer in a warm-blooded animal, such as man. According to another feature of the invention there is provided the use of a compound of the formula I, or a pharmaceutically acceptable salt or prodrug thereof in combination with an anti-tumour agent selected from one listed under (i)-(ix) herein above, in the manufacture of a medicament for use in the production of an a5b1 antagonistic effect in a warm-blooded animal, such as man. According to another feature of the invention there is provided the use of a compound of the formula I, or a pharmaceutically acceptable salt or prodrug thereof in combination with an anti-tumour agent selected from one listed under (i)-(ix) herein above, in the manufacture of a medicament for use as an antiangiogenic agent in the treatment of a solid tumour in a warm-blooded animal, such as man. According to another feature of the invention there is provided the use of a compound of the formula I, or a pharmaceutically acceptable salt or prodrug thereof in combination with an anti-tumour agent selected from one listed under (i)-(ix) herein above, in the manufacture of a medicament for use in the treatment or prophylaxis of neoplastic disease such as carcinoma of the breast, ovary, lung (including small cell lung cancer, non-small cell lung cancer and bronchioalveolar cancer), colon, rectum, prostate, bile duct, bone, bladder, head and neck, kidney, liver, gastrointestinal tissue, oesophagus, pancreas, skin, testes, thyroid, uterus, cervix, vulva or other tissues, as well as leukemias and lymphomas including CLL and CML, tumors of the central and peripheral nervous system, and other tumor types such as melanoma, multiple myeloma, fibrosarcoma and osteosarcoma, and malignant brain tumors in a warm-blooded animal, such as man.

According to another feature of the invention there is provided a compound of the formula I, or a pharmaceutically acceptable salt or prodrug thereof in combination with an anti-tumour agent selected from one listed under (i)-(ix) herein above for use in the treatment of a cancer in a warm-blooded animal, such as man. According to another feature of the invention there is provided a compound of the formula I, or a pharmaceutically acceptable salt or prodrug thereof in combination with an anti-tumour agent selected from one listed under (i)-(ix) herein above for use in the production of an a5b1 antagonistic effect in a warm-blooded animal, such as man. According to another feature of the invention there is provided a compound of the formula I, or a pharmaceutically acceptable salt or prodrug thereof in combination with an anti-tumour agent selected from one listed under (i)-(ix) herein above for use as an antiangiogenic agent in the treatment of a solid tumour in a warm-blooded animal, such as man. According to another feature of the invention there is provided a compound of the formula I, or a pharmaceutically acceptable salt or prodrug thereof in combination with an anti-tumour agent selected from one listed under (i)-(ix) herein above for use in the treatment or prophylaxis of neoplastic disease such as carcinoma of the breast, ovary, lung (including small cell lung cancer, non-small cell lung cancer and bronchioalveolar cancer), colon, rectum, prostate, bile duct, bone, bladder, head and neck, kidney, liver, gastrointestinal tissue, oesophagus, pancreas, skin, testes, thyroid, uterus, cervix, vulva or other tissues, as well as leukemias and lymphomas including CLL and CML, tumors of the central and peripheral nervous system, and other tumor types such as melanoma, multiple myeloma, fibrosarcoma and osteosarcoma, and malignant brain tumors in a warm-blooded animal, such as man.

Therefore in an additional feature of the invention, there is provided a method of treating a cancer in a warm-blooded animal, such as man, in need of such treatment which comprises administering to said animal an effective amount of a compound of formula I, or a pharmaceutically acceptable salt or prodrug thereof in combination with an anti-tumour agent selected from one listed under (i)-(ix) herein above. In an additional feature of the invention, there is provided the production of an a5b1 antagonistic effect in a warm-blooded animal, such as man, in need of such treatment which comprises administering to said animal an effective amount of a compound of formula I, or a pharmaceutically acceptable salt or prodrug thereof in combination with an anti-tumour agent selected from one listed under (i)-(ix) herein above. In an additional feature of the invention, there is provided a method of treating pathogenic angiogenesis in a warm-blooded animal, such as man, in need of such treatment which comprises administering to said animal an effective amount of a compound of formula I, or a pharmaceutically acceptable salt or prodrug thereof in combination with an anti-tumour agent selected from one listed under (i)-(ix) herein above. In an additional feature of the invention, there is provided a method of treating neoplastic disease such as carcinoma of the breast, ovary, lung (including small cell lung cancer, non-small cell lung cancer and bronchioalveolar cancer), colon, rectum, prostate, bile duct, bone, bladder, head and neck, kidney, liver, gastrointestinal tissue, oesophagus, pancreas, skin, testes, thyroid, uterus, cervix, vulva or other tissues, as well as leukaemias and lymphomas including CLL and CML, tumours of the central and peripheral nervous system, and other tumour types such as melanoma, multiple myeloma, fibrosarcoma and osteosarcoma, and malignant brain tumours in a warm-blooded animal, such as man, in need of such treatment which comprises administering to said animal an effective amount of a compound of formula I, or a pharmaceutically acceptable salt or prodrug thereof in combination with an anti-tumour agent selected from one listed under (i)-(ix) herein above. According to a further aspect of the present invention there is provided a kit comprising a compound of formula I, or a pharmaceutically acceptable salt or prodrug thereof in combination with an anti-tumour agent selected from one listed under (i)-(ix) herein above.

According to a further aspect of the present invention there is provided a kit comprising:

a) a compound of formula I, or a pharmaceutically acceptable salt or prodrug thereof in a first unit dosage form;
b) an anti-tumour agent selected from one listed under (i)-(ix) herein above; in a second unit dosage form; and
c) container means for containing said first and second dosage forms.

### EXAMPLES

The invention will now be illustrated in the following Examples in which, generally:

(i) operations were carried out at ambient temperature, i.e. in the range 17 to 25° C. and under an atmosphere of an inert gas such as nitrogen or argon unless otherwise stated;

(ii) in general, the course of reactions was followed by thin layer chromatography (TLC) and/or analytical high pressure liquid chromatography (HPLC); the reaction times that are given are not necessarily the minimum attainable;

(iii) when necessary, organic solutions were dried over anhydrous magnesium sulfate, work-up procedures were carried out using traditional layer separating techniques, evaporations were carried out either by rotary evaporation in vacuo or in a Genevac HT-4/EZ-2.

(iv) yields, where present, are not necessarily the maximum attainable, and when necessary, reactions were repeated if a larger amount of the reaction product was required;

(v) in general, the structures of the end-products of the formula I were confirmed by nuclear magnetic resonance (NMR) and/or mass spectral techniques; electrospray mass spectral data were obtained using a Waters ZMD or Waters ZQ LC/mass spectrometer acquiring both positive and negative ion data, generally, only ions relating to the parent structure are reported; proton NMR chemical shift values were measured on the delta scale using a Bruker Advance operating at 500 MHz. The following abbreviations have been used: s, singlet; d, doublet; t, triplet; q, quartet; m, multiplet; br, broad;

(vi) unless stated otherwise compounds containing an asymmetric carbon and/or sulfur atom were not resolved;

(vii) intermediates were not necessarily fully purified but their structures and purity were assessed by TLC, analytical HPLC, infra-red (IR) and/or NMR analysis;

(viii) unless otherwise stated, column chromatography (by the flash procedure) and medium pressure liquid chromatography (MPLC) were performed on Merck Kieselgel silica (Art. 9385);

(ix) the following analytical HPLC methods were used; in general, reversed-phase silica was used with a flow rate of about 1 ml per minute and detection was by Electrospray Mass Spectrometry and by UV absorbance at a wavelength of 254 nm;

(x) where certain compounds were obtained as an acid-addition salt, for example a mono-hydrochloride salt or a di-hydrochloride salt, the stoichiometry of the salt was based on the number and nature of the basic groups in the compound, the exact stoichiometry of the salt was generally not determined, for example by means of elemental analysis data;

(xi) the following abbreviations have been used:
AcOEt: ethyl acetate
DIPEA: diisopropylethylamine
DCM: dichloromethane
DMF: N,N-dimethylformamide
TBTU: O-Benzotriazol-1-yl-N,N,N′,N′-tetramethyluronium tetrafluoroborate
TEA Triethylamine
THF Tetrahydrofuran

Preparative HPLC was performed on C18 reverse phase-silica, for example on a Waters ‘Xterra’ preparative reverse-phase column (5 microns silica, 19 mm diameter, 100 mm length) using decreasingly polar mixtures as eluent, for example decreasingly polar mixtures of water containing 1% acetic acid (acidic conditions) or (NH₄)₂CO₃ (4 g/l) (basic conditions) and acetonitrile.

### Example 1.1

### N-[(3,5-dimethylisoxazol-4-yl)carbonyl]-3-(5-{3-[6-(methylamino)pyridin-2-yl]propyl}thiophen-2-yl)-L-alanine

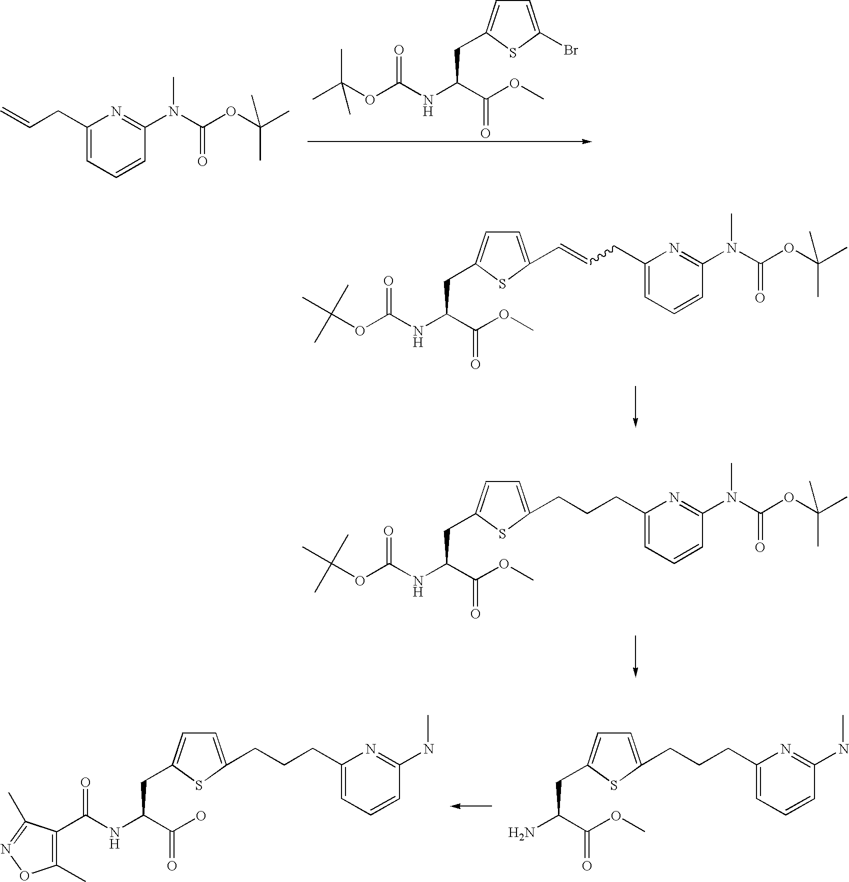

To a solution of methyl 3-(5-{3-[6-(methylamino)pyridin-2-yl]propyl}-2-thienyl)-L-alaninate (190 mg, 0.57 mmol) in DMF (1.5 ml) were added TBTU (259 mg, 0.68 mmol) and a solution of 3,5-dimethylisoxazole-4-carboxylic acid (80 mg, 0.57 mmol) in DMF (6 ml). The reaction mixture was allowed to stir at room temperature for 48 hours. NaOH 6N (15 drops) was then added. After 3 hours at room temperature, the crude mixture was filtered and purified by C18 reverse phase chromatography (basic conditions) to afford the title compound as a pale yellow solid (190 mg, 76%).

¹H NMR Spectrum (DMSO-d6) 1.87-1.96 (m, 2H), 2.18 (s, 3H), 2.39 (s, 3H), 2.53 (t, 2H), 2.70-2.77 (m, 5H), 3.16 (dd, 1H), 3.33 (dd, 1H), 4.51 (ddd, 1H), 6.23 (d, 1H), 6.30 (d, 1H), 6.33 (bs, 1H), 6.65 (d, 1H), 6.72 (d, 1H), 7.27 (dd, 1H), 8.28 (d, 1H)

Mass Spectrum [M+H]⁺=443

The methyl 3-(5-{3-[6-(methylamino)pyridin-2-yl]propyl}-2-thienyl)-L-alaninate used as the starting material was prepared as follows:

A solution of methyl 3-(5-bromo-2-thienyl)-N-(tert-butoxycarbonyl)-L-alaninate (1.2 g, 3.3 mmol) (described in US 2005/0171148, example E5), tert-butyl (6-allylpyridin-2-yl)methylcarbamate (817 mg, 3.3 mmol), Palladium(II) acetate (222 mg, 1.0 mmol), tri-O-tolylphosphine (401 mg, 1.3 mmol) and DIPEA (0.57 ml, 3.3 mmol) in acetonitrile (4 ml) was degassed, then sealed and heated to 110° C. for 3 hours in microwave. The reaction mixture was cooled down, filtered to remove the catalyst and purified by silica gel flash chromatography (0 to 20% ethyl acetate in petroleum ether) to afford methyl N-(tert-butoxycarbonyl)-3-[5-(3-{6-[(tert-butoxycarbonyl)(methyl)amino]pyridin-2-yl}prop-1-en-1-yl)-2-thienyl]-L-alaninate as a cis-trans mixture (640 mg, 37%); Mass Spectrum [M+H]⁺=532.

A mixture of methyl N-(tert-butoxycarbonyl)-3-[5-(3-{6-[(tert-butoxycarbonyl)(methyl)amino]pyridin-2-yl}prop-1-en-1-yl)-2-thienyl]-L-alaninate (640 mg, 1.20 mmol) and Pd/C 10% (120 mg) in ethanol (30 ml) was hydrogenated under 3 bars of H₂ for 3 hours, filtered through celite and concentrated to afford methyl N-(tert-butoxycarbonyl)-3-[5-(3-{6-[(tert-butoxycarbonyl)(methyl)amino]pyridin-2-yl}propyl)-2-thienyl]-L-alaninate as a colorless oil (640 mg, 99%); ¹H NMR Spectrum (DMSO-d6) 1.35 (s, 9H), 1.45 (s, 9H), 1.92-2.02 (m, 2H), 2.72 (t, 2H), 2.75 (t, 2H), 3.02 (dd, 1H), 3.15 (dd, 1H), 3.28 (s, 3H), 3.62 (s, 3H), 4.13 (ddd, 1H), 6.65 (d, 1H), 6.69 (d, 1H), 6.97 (d, 1H), 7.32 (d, 1H), 7.41 (d, 1H), 7.65 (dd, 1H).

TFA (5 ml) was added to a solution of methyl N-(tert-butoxycarbonyl)-3-[5-(3-{6-[(tert-butoxycarbonyl)(methyl)amino]pyridin-2-yl}propyl)-2-thienyl]-L-alaninate (640 mg, 1.22 mmol) in dichloromethane (4 ml). The reaction mixture was stirred 1 hour at room temperature, then solvent was removed. The residue was dissolved in dichloromethane and NH₃/MeOH 7N was added. Filtration and evaporation were followed by a purification by silica gel flash chromatography (0 to 5% MeOH in dichloromethane) to afford methyl 3-(5-{3-[6-(methylamino)pyridin-2-yl]propyl}-2-thienyl)-L-alaninate as a gum (350 mg, 88%); Mass Spectrum [M+H]⁺=334; ¹H NMR Spectrum (DMSO-d6) 1.88-1.96 (m, 2H), 1.97 (bs, 2H), 2.54 (t, 2H), 2.73 (d, 3H), 2.93 (dd, 1H), 3.01 (dd, 1H), 3.53 (t, 1H), 3.60 (s, 3H), 6.22 (d, 1H), 6.29 (q, 1H), 6.32 (d, 1H), 6.62-6.65 (m, 2H), 7.27 (dd, 1H).

The tert-butyl (6-allylpyridin-2-yl)methylcarbamate used in the above reaction was prepared as follows:

A solution of tert-butyl[6-bromopyridin-2-yl]methylcarbamate (800 mg, 2.79 mmol, described in WO2004/113331), 2-allyl-4,4,5,5-tetramethyl-1,3,2-dioxaborolane (0.94 ml, 3.07 mmol), Pd(PPh₃)₄ (312 mg, 0.28 mmol) and cesium fluoride (1.26 g, 8.36 mmol) in THF (3 ml) was degassed, then heated to 80° C. for 1 hour. The reaction mixture was cooled down, diluted in dichloromethane (100 ml), washed with water and dried with MgSO₄. After concentration, a purification by silica gel flash chromatography (50-50 heptane/dichloromethane) afforded tert-butyl (6-allylpyridin-2-yl)methylcarbamate as a yellow oil (540 mg, 79%); ¹H NMR Spectrum (DMSO-d6) 1.45 (s, 9H), 3.27 (s, 3H), 3.47 (d, 2H), 5.09 (ddd, 1H), 5.13 (ddd, 1H), 5.99-6.10 (m, 1H), 6.98 (d, 1H), 7.42 (d, 1H), 7.67 (t, 1H).

### Example 1.2

The procedure described above for example 1.1 was repeated by coupling the appropriate acid with methyl 3-(5-{3-[6-(methylamino)pyridin-2-yl]propyl}-2-thienyl)-L-alaninate. Thus were obtained the compounds shown in Table 1:

TABLE 1

| | | | | Mass | ¹H NMR Data |
|---|---|---|---|---|---|
| Example | Acid | Name | Yield | Ion | (500 MHz) |
| 1.2 | 3,5- | N-[(3,5- | 76% | 493 [M + H]⁺ | (DMSOd6) |
| | dichloro- | dichloropyridin-4- | | | 1.84-1.98 (m, 2H), |
| | 4-pyridine | yl)carbonyl]-3-(5-{3- | | | 2.55 (t, 2H), |
| | corboxylic | [6- | | | 2.69-2.77 (m, |
| | acid | (methylamino)pyridin- | | | 5H), 3.12 (dd, |
| | | 2-yl]propyl}thiophen- | | | 1H), 3.28 (dd, |
| | | 2-yl)-L-alanine | | | 1H), 4.61 (ddd, |
| | | | | | 1H), 6.22 (d, |
| | | | | | 1H), |
| | | | | | 6.29-6.35 (m, 2H), 6.64 (d, |
| | | | | | 1H), 6.72 (d, |
| | | | | | 1H), 7.28 (dd, |
| | | | | | 1H), 8.63 (s, |
| | | | | | 2H), 9.18 (d, 1H) |

### Example 2

### N-[(3,5-dimethylisoxazol-4-yl)carbonyl]-3-{5-[3-(5,6,7,8-tetrahydro-1,8-naphthyridin-2-yl)propyl]thiophen-2-yl}-L-alanine

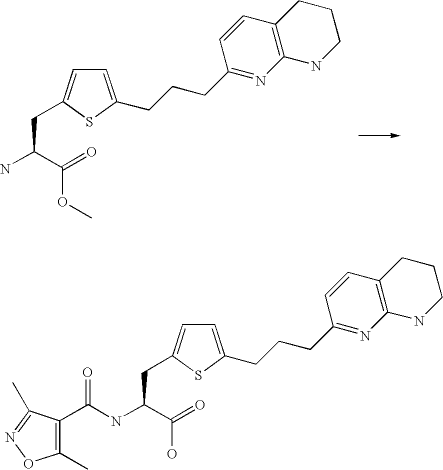

O-benzotriazol-1-yl-N,N,N′,N′-tetramethyluronium tetrafluoroborate (268 mg, 0.83 mmol), 4-methylmorpholine (0.084 ml, 0.76 mmol) and 3,5-dimethylisoxazole-4-carboxylic acid (98 mg, 0.70 mmol) were added under an argon atmosphere to a stirred solution of methyl 3-{5-[3-(5,6,7,8-tetrahydro-1,8-naphthyridin-2-yl)propyl]thiophen-2-yl}-L-alaninate (250 mg, 0.70 mmol) dissolved in DMF (2.5 mL). The resulting solution was stirred at 25° C. for 2 hours. Then NaOH 6N (0.464 ml, 2.78 mmol) was added to the stirred mixture. The resulting solution was stirred at 25° C. for 1 hour. The reaction mixture was purified by C18 reverse phase chromatography (basic conditions). The fractions containing the desired compound were evaporated to dryness to afford the title compound as a beige solid (157 mg, 48.2%); Mass spectrum [M+H]⁺=467; ¹H NMR Spectrum (DMSOd6) 1.70-1.78 (m, 2H), 1.81-1.90 (m, 2H), 2.18 (s, 3H), 2.39 (s, 3H), 2.45 (t, 2H), 2.60 (t, 2H), 2.70 (t, 2H), 3.15 (dd, 1H), 3.20-3.26 (m, 2H), 3.32 (dd partially hidden by H₂O, 1H), 4.49 (ddd, 1H), 6.23 (d, 1H), 6.42 (bs, 1H), 6.64 (d, 1H), 6.71 (d, 1H), 7.03 (d, 1H), 8.27 (d, 1H).

The methyl 3-{5-[3-(5,6,7,8-tetrahydro-1,8-naphthyridin-2-yl)propyl]thiophen-2-yl}-L-alaninate starting material was prepared as follows:

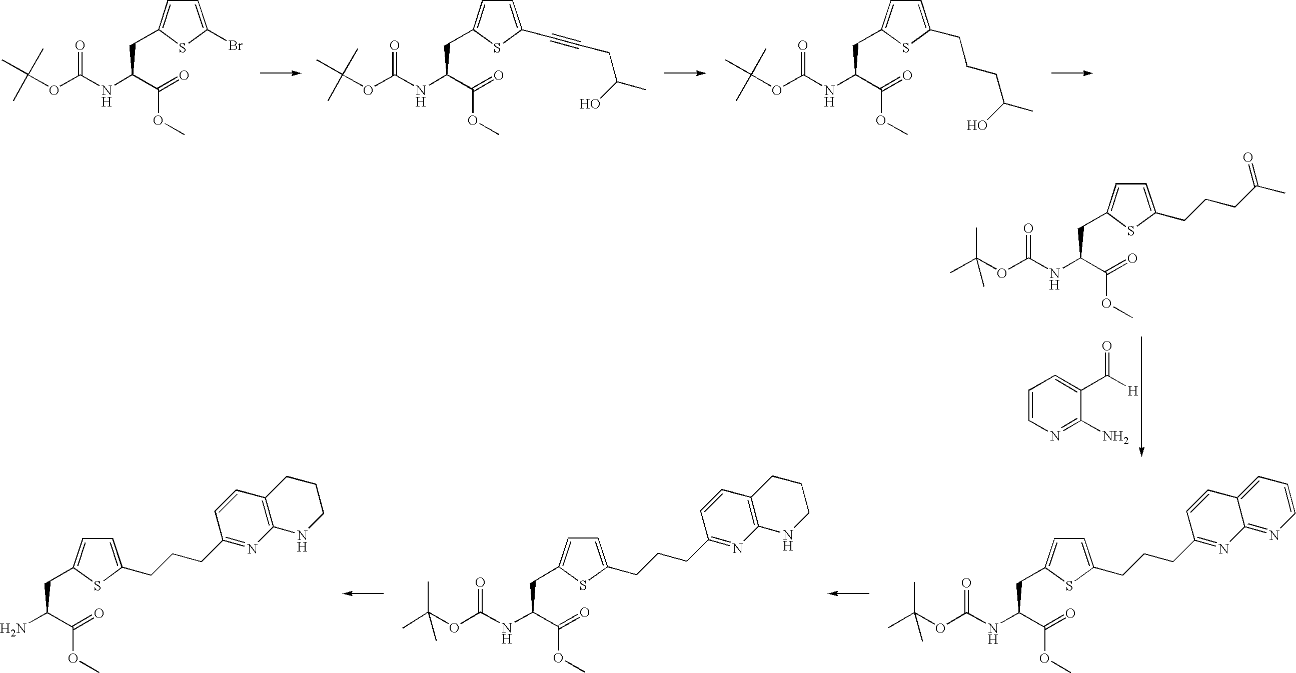

A suspension of methyl 3-(5-bromo-2-thienyl)-N-(tert-butoxycarbonyl)-L-alaninate (7.25 g, 19.9 mmol, described in US2005/171148, Example E5), bis(triphenylphosphine) palladium (II) chloride (835 mg, 1.19 mmol), CuI (152 mg, 0.8 mmol), 4-pentyn-2-ol (3.74 ml, 39.8 mmol) and TEA (11.09 ml, 79.6 mmol) in DMF (60 ml) was stirred at 80° C. under nitrogen for 1 hour 30 minutes. The mixture was extracted with ethyl acetate and purified by silica gel flash chromatography (20 to 80% ethyl acetate in petroleum ether) to afford methyl N-(tert-butoxycarbonyl)-3-[5-(4-hydroxypent-1-yn-1-yl)thiophen-2-yl]-L-alaninate (7 g, 96%); Mass spectrum [M+H]⁺=368; ¹H NMR Spectrum: (DMSOd6) 1.15 (d, 3H), 1.36 (s, 9H), 2.44 (dd, 1H), 2.53 (dd, partially hidden by DMSOd5, 1H), 3.04 (dd, 1H), 3.20 (dd, 1H), 3.63 (s, 3H), 3.77-3.85 (m, 1H), 4.15 (ddd, 1H), 4.85 (d, 1H), 6.81 (d, 1H), 7.02 (d, 1H), 7.39 (d, 1H).

A mixture of methyl N-(tert-butoxycarbonyl)-3-[5-(4-hydroxypent-1-yn-1-yl)thiophen-2-yl]-L-alaninate (11 g, 30.2 mmol) and Pd/C 10% (700 mg) in methanol (100 ml) was hydrogenated under 4 bar of H₂ for 3 hours. Pd/C 10% (700 mg) was added to and the mixture was hydrogenated further for 4 hours then filtered through celite, evaporated and purified by silica gel flash chromatography (20 to 80% ethyl acetate in petroleum ether) to afford methyl N-(tert-butoxycarbonyl)-3-[5-(4-hydroxypentyl)thiophen-2-yl]-L-alaninate (8.9 g, 80%); ¹H NMR Spectrum (DMSOd6) 1.03 (d, 3H), 1.27-1.43 (m, 11H), 1.50-1.71 (m, 2H), 2.63-2.77 (m, 2H), 3.02 (dd, 1H), 3.15 (dd, 1H), 3.59 (ddd, 1H), 3.63 (s, 3H), 4.09-4.19 (m, 1H), 4.37 (d, 1H), 6.63 (d, 1H), 6.69 (d, 1H), 7.32 (d, 1H).

A solution of methyl N-(tert-butoxycarbonyl)-3-[5-(4-hydroxypentyl)thiophen-2-yl]-L-alaninate (8.37 g, 22.53 mmol) and pyridinium dichromate (25.43 g, 67.6 mmol) in DCM (250 ml) was stirred at ambient temperature for 24 hours. After filtration to remove solids and extraction with DCM, the residue was purified by silica gel flash chromatography (15 to 30% ethyl acetate in petroleum ether) to afford methyl N-(tert-butoxycarbonyl)-3-[5-(4-oxopentyl)thiophen-2-yl]-L-alaninate (5.65 g, 68%); ¹H NMR Spectrum (DMSOd6) 1.37 (s, 9H), 1.70-1.80 (m, 2H), 2.07 (s, 3H), 2.47 (t, 2H), 2.68 (t, 2H), 3.02 (dd, 1H), 3.15 (dd, 1H), 3.63 (s, 3H), 4.14 (ddd, 1H), 6.63 (d, 1H), 6.70 (d, 1H), 7.33 (d, 1H).

To a solution of methyl N-(tert-butoxycarbonyl)-3-[5-(4-oxopentyl)thiophen-2-yl]-L-alaninate (2.05 g, 16.79 mmol) in ethanol (130 ml) were added 2-aminopyridine-3-carboxaldehyde (5.58 g, 15.1 mmol) and L-proline (0.966 g, 8.39 mmol). The mixture was stirred overnight under argon atmosphere. After evaporation, the residue was taken up in DCM, washed with saturated NaHCO₃ and purified by silica gel flash chromatography (2 to 5% methanol in DCM) to afford methyl N-(tert-butoxycarbonyl)-3-{5-[3-(1,8-naphthyridin-2-yl)propyl]thiophen-2-yl}-L-alaninate (4.32 g, 56%); Mass spectrum [M+H]⁺=456; ¹H NMR Spectrum (DMSOd6) 1.34 (s, 9H), 2.07-2.17 (m, 2H), 2.83 (t, 2H), 2.98-3.08 (m, 3H), 3.16 (dd, 1H), 3.63 (s, 3H), 4.14 (ddd, 1H), 6.67-6.74 (m, 2H), 7.33 (d, 1H), 7.55 (d, 1H), 7.58 (dd, 1H), 8.37 (d, 1H), 8.42 (dd, 1H), 9.03 (dd, 1H).

A suspension of N-(tert-butoxycarbonyl)-3-{5-[3-(1,8-naphthyridin-2-yl)propyl]thiophen-2-yl}-L-alaninate (3.8 g, 8.34 mmol) and Pd/C 10% (0.335 g) in EtOH (200 ml) was hydrogenated under 4 atmosphere for 3 hours. The resulting solution was filtered and the filtrate was concentrated to dryness to afford the crude methyl N-(tert-butoxycarbonyl)-3-{5-[3-(5,6,7,8-tetrahydro-1,8-naphthyridin-2-yl)propyl]thiophen-2-yl}-L-alaninate (3.00 g, 78%) as a pale colourless liquid; Mass spectrum [M+H]⁺=460; ¹H NMR Spectrum: (DMSOd6) 1.36 (s, 9H), 1.72-1.80 (m, 2H), 1.83-1.93 (m, 2H), 2.46 (t, 2H), 2.60 (t, 2H), 2.70 (t, 2H), 3.01 (dd, 1H), 3.14 (dd, 1H), 3.19-3.27 (m, 2H), 3.62 (s, 3H), 4.13 (ddd, 1H), 6.25 (d, 1H), 6.29 (bs, 1H), 6.63 (d, 1H), 6.68 (d, 1H), 7.03 (d, 1H), 7.32 (d, 1H).

Trifluoroacetic acid (10.06 ml, 130.55 mmol) was added under argon atmosphere to a stirred solution of N-(tert-butoxycarbonyl)-3-{5-[3-(5,6,7,8-tetrahydro-1,8-naphthyridin-2-yl)propyl]thiophen-2-yl}-L-alaninate (3 g, 6.53 mmol) in DCM (20 ml). The resulting solution was stirred at 25° C. for 3 hours. The reaction mixture was then concentrated to dryness and taken-up in DCM. An excess of a solution of NH₃/MeOH 7N was added. After evaporation to dryness, the residue was purified by flash chromatography on silica gel eluting with 5 to 10% methanol in DCM to afford methyl 3-{5-[3-(5,6,7,8-tetrahydro-1,8-naphthyridin-2-yl)propyl]thiophen-2-yl}-L-alaninate (2.400 g,) as a pale yellow gum; Mass spectrum [M+H]⁺=360; ¹H NMR Spectrum (DMSOd6) 1.70-1.79 (m, 2H), 1.83-1.92 (m, 2H), 1.97 (bs, 2H), 2.46 (t, 2H), 2.60 (t, 2H), 2.70 (t, 2H), 3.20-3.26 (m, 2H), 3.53 (t, 1H), 3.60 (s, 3H), 6.24 (d, 1H), 6.27 (bs, 1H), 6.63 (s, 2H), 7.02 (d, 1H).

### Example 3

### N-(3,5-dichloroisonicotinoyl)-3-{5-[3-(5,6,7,8-tetrahydro-1,8-naphthyridin-2-yl)propyl]-thiophen-2-yl}-L-alanine

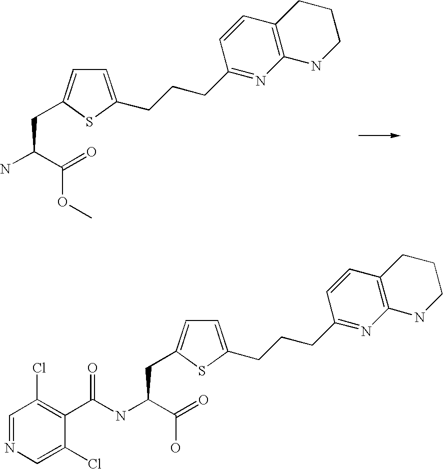

The procedure described for example 2 was repeated by coupling methyl 3-{5-[3-(5,6,7,8-tetrahydro-1,8-naphthyridin-2-yl)propyl]thiophen-2-yl}-L-alaninate with 3,5-dichloroisonicotinic acid to give the title product (Yield=34%); Mass spectrum [M+H]⁺=519; ¹H NMR Spectrum (DMSOd6+TFAd) 1.82-1.89 (m, 2H), 1.92-2.00 (m, 2H), 2.27-2.83 (m, 6H), 3.16 (dd, 1H), 3.32 (dd, 1H), 3.41-3.46 (m, 2H), 4.72 (dd, 1H), 6.61 (d, 1H), 6.69 (d, 1H), 6.78 (d, 1H), 7.61 (d, 1H), 8.65 (d, 2H).

### Example 4

### N-(3,5-dichloroisonicotinoyl)-3-{5-[2-(5,6,7,8-tetrahydro-1,8-naphthyridin-2-yl)ethoxy]-2-thienyl}-L-alanine

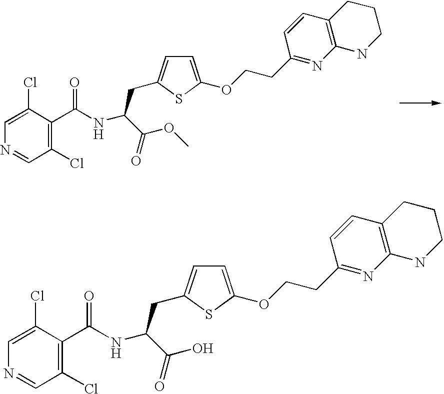

Sodium hydroxide (6N) (0.019 ml, 0.11 mmol) was added dropwise to a stirred solution of methyl N-(3,5-dichloroisonicotinoyl)-3-{5-[2-(5,6,7,8-tetrahydro-1,8-naphthyridin-2-yl)ethoxy]-2-thienyl}-L-alaninate (20 mg, 0.04 mmol) dissolved in tetrahydrofuran (1 ml) over a period of 1 minute at 0° C. The resulting solution was stirred at room temperature for 1 hour. The pH was adjusted to approximately 7 with 2 M HCl and the reaction mixture was concentrated. The resulting clear, colourless solution was diluted with water (5 ml) and the pH was reduced to 5.5 with 0.1 N HCl (0.374 ml, 0.04 mmol). The resulting think white precipitate was collected by filtration, washed with water (5×5 ml) and dried to a constant weight to afford the title compound (2.2 mg, 11%), which was a white solid. The aqueous phase also containing the title product was concentrated to dryness and the residue taken up in DMF-water (3 ml) and purified by C18 reverse phase chromatography (basic conditions). The fractions containing the desired compound were evaporated to dryness to afford the title compound (10 mg, 51%) as a white solid; Mass spectrum [M+H]⁺: 521; ¹H NMR Spectrum: DMOSd6: 1.69-1.80 (m, 2H), 2.57-2.66 (m, 2H), 2.85 (t, 2H), 3.03 (dd, 1H), 3.16 (dd, 1H), 3.21-3.27 (m, 2H), 4.23 (t, 2H), 4.55 (ddd, 1H), 6.09 (d, 1H), 6.33 (d, 1H), 6.37 (bs, 1H), 6.51 (d, 1H), 7.06 (d, 1H), 8.65 (s, 2H), 9.16 (d, 1H).

The methyl N-(3,5-dichloroisonicotinoyl)-3-{5-[2-(5,6,7,8-tetrahydro-1,8-naphthyridin-2-yl)ethoxy]-2-thienyl}-L-alaninate starting material was prepared as follows:

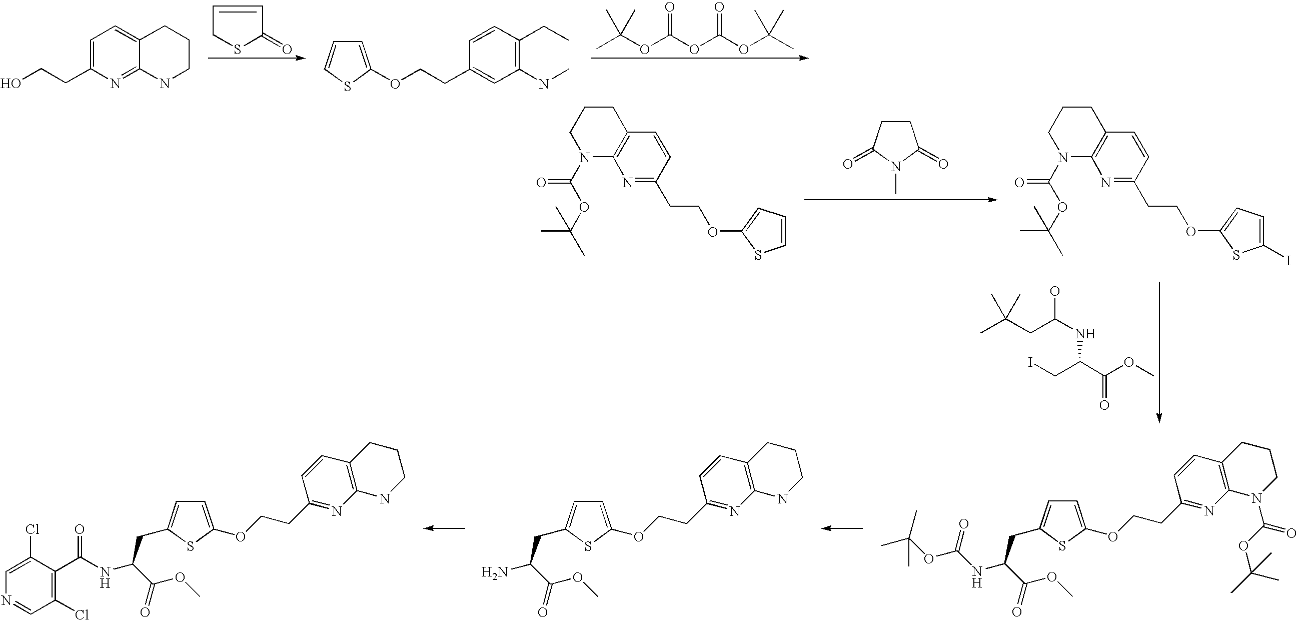

A solution of triphenylphosphine (5.24 g, 19.97 mmol) in THF (15 ml) was added dropwise under argon to a cold solution of diisopropyl azodicarboxylate (3.93 ml, 19.97 mmol) in THF (25 ml) at such a rate to maintain temperature below 5° C. (25 minutes). Five minutes after the end of addition, a voluminous cream solid precipitated. The mixture was diluted with THF (5 ml). A solution of 2-(5,6,7,8-tetrahydro-1,8-naphthyridin-2-yl)ethanol (described in WO 2004/058761, page 34, 1.78 g, 9.99 mmol) and 2(5H)-thiophenone (1.000 g, 9.99 mmol) dissolved in THF (25 ml) was added dropwise over a period of 15 minutes at 5° C. The resulting stirred mixture was allowed to warm to room temperature. After 5 hours the reaction mixture was concentrated and the dark blue/green residue was diluted with a mixture of ethyl acetate/ether to be washed once with water, then extracted with HCl 0.5N (X4). The acid solution was washed with AcOEt/Ether (X3), then was basified with solid NaHCO₃ and extracted with AcOEt/Ether (X3). The organic dark blue solution was washed with brine, dried over MgSO₄ in presence of activated charcoal, filtrated and concentrated to afford a brown oil (2.5 g) which was purified by flash chromatography on silica gel eluting with 0 to 30% of ether in DCM. The solvent was evaporated to dryness to afford a pale off-white oil which crystallised on standing (1.9 g). The solid was triturated with diethyl ether/petroleum ether to give a solid which was collected by filtration and dried under vacuum to give 7-(2-(2-thienyloxy)ethyl)-1,2,3,4-tetrahydro-1,8-naphthyridine (1.380 g, 53.1%) as a white crystalline solid; Mass Spectrum [M+H]⁺=261; ¹H NMR Spectrum: (DMSO-d6) 1.71-1.79 (m, 2H), 2.61 (t, 2H), 2.88 (t, 2H), 3.21-3.26 (m, 2H), 4.28 (t, 2H), 6.29 (dd, 1H), 6.34 (bs, 1H), 6.35 (d, 1H), 6.70-6.74 (m, 2H), 7.06 (d, 1H).

Di-tert-butyl dicarbonate (4.36 g, 19.97 mmol) and 7-(2-(2-thienyloxy)ethyl)-1,2,3,4-tetrahydro-1,8-naphthyridine (1.04 g, 3.99 mmol) in DCM (5 ml) were stirred at room temperature for 16 hours. THF (5.00 ml) was added and the reaction temperature was increased to 60° C. The reaction mixture was stirred for 2 hours. The crude product was purified by flash chromatography on silica gel eluting with 0 to 50% ethyl acetate in petroleum ether. The solvent was evaporated to dryness to afford tert-butyl 7-(2-(2-thienyloxy)ethyl)-3,4-dihydro-1,8-naphthyridine-1(2H)-carboxylate (1.432 g, 99%) as a clear colourless oil; ¹H NMR Spectrum: (DMSO-d6) 1.43 (s, 9H), 1.78-1.85 (m, 2H), 2.70 (t, 2H), 3.08 (t, 2H), 3.63 (dd, 2H), 4.38 (t, 2H), 6.30 (dd, 1H), 6.70-6.73 (m, 2H), 6.99 (d, 1H), 7.45 (d, 1H).

N-iodosuccinimide (0.906 g, 4.03 mmol) was added portionwise to a stirred solution of tert-butyl 7-(2-(2-thienyloxy)ethyl)-3,4-dihydro-1,8-naphthyridine-1(2H)-carboxylate (1.32 g, 3.66 mmol) dissolved in THF (50 ml) over a period of 5 minutes at 0° C. The resulting solution was stirred at room temperature for 5 hours. The reaction mixture was concentrated to dryness, diluted with ethyl acetate (200 ml), washed with a saturated aqueous solution of sodium hydrogencarbonate (2×20 ml), water (2×20 ml), dried over magnesium sulfate and concentrated to afford the crude product as a dark orange oil. The crude product was purified by flash chromatography on silica gel eluting with 0 to 60% ethyl acetate in petroleum ether. The solvent was evaporated to dryness to afford tert-butyl 7-{2-[(5-iodo-2-thienyl)oxy]ethyl}-3,4-dihydro-1,8-naphthyridine-1(2H)-carboxylate (1.700 g, 95%) as a pale orange oil which solidified on standing; ¹H NMR Spectrum: (DMSO-d6) 1.42 (s, 9H), 1.78-1.86 (m, 2H), 2.70 (t, 2H), 3.06 (t, 2H), 3.63 (dd, 2H), 4.38 (t, 2H), 6.11 (d, 1H), 6.97 (d, 1H), 7.00 (d, 1H), 7.45 (d, 1H).

Solid zinc (1.371 ml, 20.97 mmol) was added to an oven-dried flask and heated for 10 minutes under vacuum, purged with nitrogen and allowed to cool to room temperature. 1,2-Dibromoethane (0.090 ml, 1.05 mmol) in DMF (2 ml) at room temperature was added dropwise to the zinc. The resulting suspension was heated at 90° C. for 30 minutes, cooled to room temperature and chlorotrimethylsilane (0.026 ml, 0.21 mmol) was added in one portion. The reaction mixture was stirred at room temperature for 1 hour. L-alanine, N-[(1,1-dimethylethoxy)carbonyl]-3-iodo-, methyl ester (2 g, 6.08 mmol) in solution in DMF (2 ml) was added over a period of 1 minute at room temperature under nitrogen. The suspension was stirred at 35° C. for 2 hours under nitrogen. The resulting slurry was added at room temperature, portionwise over a period of 5 minutes to a stirred solution of tert-butyl 7-{2-[(5-iodo-2-thienyl)oxy]ethyl}-3,4-dihydro-1,8-naphthyridine-1(2H)-carboxylate (1.7 g, 3.50 mmol), tris(dibenzylideneacetone) dipalladium (0.320 g, 0.35 mmol) and dicyclohexyl(2′,6′-diisopropoxybiphenyl-2-yl)phosphine (0.652 g, 1.40 mmol) dissolved in DMF (4 ml at room temperature under nitrogen). The resulting suspension was stirred at 70° C. for 16 hours. The reaction mixture was allowed to cool to room temperature under stirring over a period of 1 hour, quenched with water and extracted with ethyl acetate (2×100 ml). The combined organic phases were washed with water (3×10 ml), dried over magnesium sulfate and concentrated to afford the crude product as a dark brown oil. The crude product was purified by flash chromatography on silica gel eluting with 0 to 100% ethyl acetate in petroleum ether. The solvent was evaporated to dryness to afford tert-butyl 7-{2-[(5-{(2S)-2-[(tert-butoxycarbonyl)amino]-3-methoxy-3-oxopropyl}-2-thienyl)oxy]ethyl}-3,4-dihydro-1,8-naphthyridine-1(2H)-carboxylate (0.205 g, 10.4%); Mass Spectrum [M+H]⁺=562.

tert-Butyl 7-{2-[(5-{(2S)-2-[(tert-butoxycarbonyl)amino]-3-methoxy-3-oxopropyl}-2-thienyl)oxy]ethyl}-3,4-dihydro-1,8-naphthyridine-1(2H)-carboxylate (0.2 g, 0.36 mmol) was dissolved in DCM (1 ml) cooled to 0° C. and treated with trifluoroacetic acid (5 ml). The resulting solution was stirred at room temperature for 1 hour. The resulting mixture was evaporated to dryness and the residual trifluoroacetic acid was removed by azeotropic distillation with toluene under vacuum. The crude product was purified by flash chromatography on silica gel eluting with 0 to 5% methanolic ammonia (7 N) in DCM. The solvent was evaporated to dryness to afford methyl 3-{5-[2-(5,6,7,8-tetrahydro-1,8-naphthyridin-2-yl)ethoxy]-2-thienyl}-L-alaninate (0.100 g, 78%) as a clear tan oil; Mass Spectrum [M+H]⁺=362; ¹H NMR Spectrum: (CDCl₃) 1.89-1.97 (m, 2H), 2.70-2.78 (m, 2H), 2.98 (dd, 1H), 3.06-3.13 (m, 3H), 3.45-3.51 (m, 2H), 3.65 (dd, 1H), 3.74 (s, 3H), 4.30 (t, 2H), 6.05 (d, 1H), 6.43-6.47 (m, 2H), 7.25 (bs partially hidden by CHCl3, 1H).

Solid O-benzotriazol-1-yl-N,N,N′,N′-tetramethyluronium tetrafluoroborate (0.042 g, 0.13 mmol) was added portionwise to a stirred solution of methyl 3-{5-[2-(5,6,7,8-tetrahydro-1,8-naphthyridin-2-yl)ethoxy]-2-thienyl}-L-alaninate (0.043 g, 0.12 mmol), 3,5-dichloroisonicotinic acid (0.022 g, 0.11 mmol) and 4-methylmorpholine (0.016 ml, 0.14 mmol) dissolved in DCM (100 ml) over a period of 10 minutes at 0° C. The resulting solution was stirred at 25° C. for 2 days. The reaction mixture was basified with a saturated aqueous solution of sodium hydrogencarbonate and extracted with DCM (1×100 ml). The combined organic phases were washed with water (2×20 ml), dried over magnesium sulfate and concentrated to afford the crude product as a clear orange oil. The crude product was purified by flash chromatography on silica gel eluting with 0 to 40% ethyl acetate in petroleum ether. The solvent was evaporated to dryness to afford methyl N-(3,5-dichloroisonicotinoyl)-3-{5-[2-(5,6,7,8-tetrahydro-1,8-naphthyridin-2-yl)ethoxy]-2-thienyl}-L-alaninate (31.4%) as a white crystalline solid.

Mass Spectrum [M+H]⁺: 535.

1. A compound of formula I: 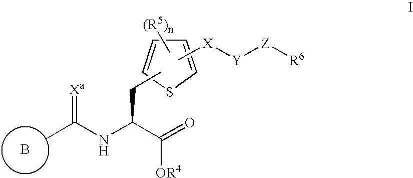
wherein:
X^{a} is selected from oxygen or sulphur;
B is heteroaryl which is substituted ortho to the C(X^{a}) group in formula I by R¹ and optionally bears one or more R³ substituents, wherein R¹ and each R³ are independently selected from halo, trifluoromethyl, cyano, isocyano, nitro, hydroxy, mercapto, amino, formyl, carboxy, carbamoyl, sulfamoyl, halo-(1-3C)alkyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,
or from a group of the formula:
Q¹-X¹—
wherein X¹ is a direct bond or is selected from O, S, SO, SO₂, N(R⁷), C(O), CH(OR⁷), C(O)N(R⁷), N(R⁷)C(O), SO₂N(R⁷), N(R⁷)SO₂, OC(R⁷)₂, SC(R⁷)₂ and N(R⁷)C(R⁷)₂,
wherein R⁷ is hydrogen or (1-6C)alkyl, and Q¹ is aryl, aryl-(1-6C)alkyl, (3-7C)cycloalkyl, (3-7C)cycloalkyl-(1-6C)alkyl, (3-7C)cycloalkenyl, (3-7C)cycloalkenyl-(1-6C)alkyl, heteroaryl, heteroaryl-(1-6C)alkyl, heterocyclyl or heterocyclyl-(1-6C)alkyl,
and wherein any carbon containing substituent on ring B optionally bears on carbon one or more R⁸ groups,
and wherein if any heteroaryl or heterocyclyl group which is a substituent on ring B contains an —NH— moiety, the nitrogen of said moiety optionally bears a group selected from R⁹,
and wherein any heterocyclyl group which is a substituent on ring B optionally bears 1 or 2 oxo or thioxo substituents;
or two adjacent substituents on ring B optionally form a (1-3C)alkylenedioxy group;
and wherein ring B is linked to the C(X^{a}) group by a ring carbon atom;
R⁴ is selected from hydrogen, (1-6C)alkyl, aryl, aryl-(1-6C)alkyl, heterocyclyl, heteroaryl, heterocyclyl(1-6)alkyl and heteroaryl-(1-6C)alkyl, which optionally bears on carbon one or more R²¹ substituents, which may be the same or different,
and wherein if any heteroaryl or heterocyclyl group within R⁴ contains an —NH— moiety, the nitrogen of said moiety optionally bears a group selected from R²²,
and wherein and wherein any heterocyclyl group within R⁴ optionally bears 1 or 2 oxo or thioxo substituents;
n is 0, 1 or 2;
each R⁵, which may be the same or different, is selected from halo, trifluoromethyl, cyano, isocyano, nitro, hydroxy, mercapto, amino, formyl, carboxy, carbamoyl, sulfamoyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,
or from a group of the formula:
Q⁵-X⁷—
wherein X⁷ is a direct bond or is selected from O, S, SO, SO₂, N(R²³), C(O), CH(OR²³), C(O)N(R²³), N(R²³)C(O), SO₂N(R²³), N(R²³)SO₂, OC(R²³)₂, SC(R²³)₂ and N(R²³)C(R²³)₂, wherein R²³ is hydrogen or (1-6C)alkyl, and Q⁵ is aryl, aryl-(1-6C)alkyl, (3-7C)cycloalkyl, (3-7C)cycloalkyl-(1-6C)alkyl, (3-7C)cycloalkenyl, (3-7C)cycloalkenyl-(1-6C)alkyl, heteroaryl, heteroaryl-(1-6C)alkyl, heterocyclyl or heterocyclyl-(1-6C)alkyl,
and wherein R⁵ optionally bears on carbon one or more R²⁴ groups,
and wherein any if any heteroaryl or heterocyclyl group within R⁵ contains an —NH— moiety, the nitrogen of said moiety optionally bears a group selected from R²⁵,
and wherein any heterocyclyl group within R⁵ optionally bears 1 or 2 oxo or thioxo substituents;
or two R⁵ substituents optionally form a (1-3C)alkylenedioxy group;
with the proviso that when X⁷ is a direct bond then Q⁵ is not aryl or heteroaryl;
X and Z, which may be the same or different, are selected from a direct bond, N(R²⁶), O, S, SO, SO₂, C(O), CH(OR²⁶), C(O)N(R²⁶), N(R²⁶)C(O), SO₂N(R²⁶), N(R²⁶)SO₂, (1-6C)alkylene, CH═CH and C≡C, wherein R²⁶ is hydrogen, (1-6C)alkyl or (3-7C)cycloalkyl;
Y is selected from (1-6C)alkylene, (3-7C)cycloalkylene and (3-7C)cycloalkenylene,
and wherein adjacent carbon atoms in any (2-6C)alkylene chain within an X, Y or Z substituent are optionally separated by the insertion into the chain of a group selected from O, S, SO, SO₂, N(R^{27a}), C(O), CH(OR²⁷), C(O)N(R²⁷), N(R²⁷)C(O), SO₂N(R²⁷), N(R²⁷)SO₂, CH═CH and C≡C wherein R²⁷ is hydrogen, (1-6C)alkyl or (3-7C)cycloalkyl, and R^{27a} is hydrogen, (1-6C)alkyl or (3-7C)cycloalkyl, (1-3C)alkoxy(1-3C)alkyl, C(O)R^{27b}S(O)R^{27b} or S(O)₂R^{27b} where R^{27b} is hydrogen, (1-3C)alkyl, (3-7C)cycloalkyl, (1-3C)alkoxy(1-3C)alkyl;
and wherein any X, Y or Z optionally bears on carbon one or more R²⁸ substituents,
R⁶ is heteroaryl, which heteroaryl contains at least one —N═ ring atom,
wherein R⁶ is linked to the group Z by a carbon atom in R⁶,
and wherein R⁶ optionally bears on carbon one or more R³¹ substituents,
and wherein if R⁶ contains an —NH— moiety, the nitrogen of said moiety optionally bears a group selected from R³⁵,
and wherein:
(i) R⁶ is a bicyclic or polycyclic heteroaryl which contains at least one unsubstituted —NH— ring member in addition to the —N═ ring atom, wherein the —NH— and ═N— group in R⁶ are attached to the same bridgehead ring atom at a junction of two fused rings in R⁶; or
(ii) R⁶ is substituted in an ortho position to the —N═ atom in R⁶ by an —NHR^{31a} group; or
(iii) Z is NH and R⁶ is attached to Z by a ring carbon atom in an ortho position to the —N═ atom in R⁶;
and wherein the group Z-R⁶ has a pKa of greater than or equal to about 6;
R⁸, R²¹, R²⁴ and R²⁸ is selected from halo, trifluoromethyl, cyano, nitro, hydroxy, amino, carboxy, carbamoyl, sulfamoyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,
or from a group of the formula:
—X²—R¹⁰
wherein X² is a direct bond or is selected from O, C(O) and N(R¹¹), wherein R¹¹ is hydrogen or (1-6C)alkyl, and R¹⁰ is halo-(1-6C)alkyl, hydroxy-(1-6C)alkyl, (1-6C)alkoxy-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl and (1-6C)alkoxycarbonylamino-(1-6C)alkyl,
or from a group of the formula:
—X³-Q²
wherein X³ is a direct bond or is selected from O, S, SO, SO₂, N(R¹²), C(O), CH(OR¹²), C(O)N(R¹²), N(R¹²)C(O), SO₂N(R¹²), N(R¹²)SO₂, OC(R¹²)₂, SC(R¹²)₂ and N(R¹²)C(R¹²)₂, wherein R¹² is hydrogen or (1-6C)alkyl, and Q² is aryl, aryl-(1-6C)alkyl, (3-7C)cycloalkyl, (3-7C)cycloalkyl-(1-6C)alkyl, (3-7C)cycloalkenyl, (3-7C)cycloalkenyl-(1-6C)alkyl, heteroaryl, heteroaryl-(1-6C)alkyl, heterocyclyl or heterocyclyl-(1-6C)alkyl,
and wherein R⁸, R²¹, R²⁴ and R²⁸ independently of each other optionally bears on carbon one or more R¹³,
and wherein any if any heteroaryl or heterocyclyl group within R⁸, R²¹, R²⁴ and R²³ contains an —NH— moiety, the nitrogen of said moiety optionally bears a group selected from R¹⁴,
and wherein any heterocyclyl group within a substituent on R⁸, R²¹, R²⁴ and R²⁸ independently of each other optionally bears 1 or 2 oxo or thioxo substituents;
R⁹, R²² and R²⁵ are each independently selected from carbamoyl, sulfamoyl, (1-6C)alkyl, (2-8C)alkenyl, (2-8C)alkynyl, (1-6C)alkylsulfonyl, (1-6C)alkoxycarbonyl, N-(1-6C)alkylcarbamoyl, N,N-di-[(1-6C)alkyl]carbamoyl, (2-6C)alkanoyl, N-(1-6C)alkylsulfamoyl and N,N-di-[(1-6C)alkyl]sulfamoyl, or from a group of the formula:
—X⁴—R¹⁵
wherein X⁴ is a direct bond or is selected from C(O), SO₂, C(O)N(R¹⁶) and SO₂N(R¹⁶), wherein R¹⁶ is hydrogen or (1-6C)alkyl, and R¹⁵ is halo-(1-6C)alkyl, hydroxy-(1-6C)alkyl, (1-6C)alkoxy-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl and (1-6C)alkoxycarbonylamino-(1-6C)alkyl,
or from a group of the formula:
—X⁵-Q³
wherein X⁵ is a direct bond or is selected from C(O), SO₂, C(O)N(R¹⁷) and SO₂N(R¹⁷), wherein R¹⁷ is hydrogen or (1-6C)alkyl, and Q³ is aryl, aryl-(1-6C)alkyl, (3-7C)cycloalkyl, (3-7C)cycloalkyl-(1-6C)alkyl, (3-7C)cycloalkenyl, (3-7C)cycloalkenyl-(1-6C)alkyl, heteroaryl, heteroaryl-(1-6C)alkyl, heterocyclyl or heterocyclyl-(1-6C)alkyl,
and wherein R⁹, R²² and R²⁵ independently of each other optionally bears on carbon one or more R¹⁸,
and wherein any if any heteroaryl or heterocyclyl group within R⁹, R²² and R²⁵ contains an —NH— moiety, the nitrogen of said moiety optionally bears a group selected from R¹⁹,
and wherein any heterocyclyl group within a substituent on R⁹, R²² and R²⁵ optionally bears 1 or 2 oxo or thioxo substituents;
R¹³ and R¹⁸ are each independently selected from halo, cyano, hydroxy, carboxy, amino, (3-6C)cycloalkyl, (2-6C)alkenyl, (2-6C)alkynyl, (1-6C)alkoxy, (1-6C)alkylamino and di-[(1-6C)alkyl]amino;
R¹⁴ and R¹⁹ are each independently selected from carbamoyl, sulfamoyl, (1-6C)alkyl, (2-8C)alkenyl, (2-8C)alkynyl, (1-6C)alkylsulfonyl, N-(1-6C)alkylcarbamoyl, N,N-di-[(1-6C)alkyl]carbamoyl, (2-6C)alkanoyl, N-(1-6C)alkylsulfamoyl and N,N-di-[(1-6C)alkyl]sulfamoyl,
or from a group of the formula:
—X⁶-Q⁴
wherein X⁶ is a direct bond or is selected from C(O), SO₂, C(O)N(R²⁰) and SO₂N(R²⁰), wherein R²⁰ is hydrogen or (1-6C)alkyl, and Q⁴ is (3-7C)cycloalkyl or (3-7C)cycloalkyl-(1-6C)alkyl;
R³¹ is selected from halo, cyano, hydroxy, nitro, amino, carbamoyl, sulfamoyl, (1-6C)alkyl, (2-6C)alkenyl, (2-6C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (2-6C)alkanoyl, N-(1-6C)alkylcarbamoyl, N,N-di-[(1-6C)alkyl]carbamoyl, (1-6C)alkoxycarbonyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino or from a group of the formula:
—X⁸—R³²
wherein X⁸ is a direct bond or is selected from O, C(O) and N(R³³), wherein R³³ is hydrogen or (1-6C)alkyl, and R³² is halo-(1-6C)alkyl, hydroxy-(1-6C)alkyl, (1-6C)alkoxy-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl and (1-6C)alkoxycarbonylamino-(1-6C)alkyl,
or from a group of the formula:
—X⁹-Q⁶
wherein X⁹ is a direct bond or is selected from O, S, SO, SO₂, C(O), N(R³⁴), C(O)N(R³⁴), N(R³⁴)C(O), SO₂N(R³⁴), N(R³⁴)SO₂ wherein R³⁴ is hydrogen or (1-6C)alkyl, and Q6 is aryl, aryl-(1-6C)alkyl, (3-7C)cycloalkyl, (3-7C)cycloalkyl-(1-6C)alkyl, heteroaryl, heteroaryl-(1-6C)alkyl, heterocyclyl or heterocyclyl-(1-6C)alkyl,
and wherein R³¹ optionally bears on carbon one or more R³⁶,
and wherein any if any heteroaryl or heterocyclyl group within R³¹ contains an —NH— moiety, the nitrogen of said moiety optionally bears a group selected from R³⁷,
and wherein any heterocyclyl group within a substituent on R³¹ optionally bears 1 or 2 oxo or thioxo substituents;
R^{31a} is selected from hydrogen, (1-6C)alkyl, (2-8C)alkenyl, (2-8C)alkynyl,
halo-(1-6C)alkyl, hydroxy-(2-6C)alkyl, (1-6C)alkoxy-(2-6C)alkyl, amino-(2-6C)alkyl, (1-6C)alkylamino-(2-6C)alkyl, di-[(1-6C)alkyl]amino-(2-6C)alkyl, (3-7C)cycloalkyl and (3-7C)cycloalkyl-(1-6C)alkyl,
and wherein any (3-7C)cycloalkyl in R^{31a} optionally bears 1 or more (1-6C)alkyl substituents;
R³⁵ and R³⁷ are selected from (1-6C)alkyl, (2-8C)alkenyl, (2-8C)alkynyl, (1-6C)alkylsulfonyl and (2-6C)alkanoyl,
or from a group of the formula:
—X¹⁰-Q⁷
wherein X¹⁰ is a direct bond or is selected from C(O), SO₂, wherein Q⁷ is (3-7C)cycloalkyl or (3-7C)cycloalkyl-(1-6C)alkyl;
R³⁶ is selected from halo, cyano, hydroxy, amino, (1-6C)alkyl, (2-6C)alkenyl, (2-6C)alkynyl, (3-6)cycloalkyl, (1-6C)alkoxy, (1-6C)alkylamino and di-[(1-6C)alkyl]amino;
or a pharmaceutically acceptable salt thereof.

2. A compound according to claim 1 of formula IA: 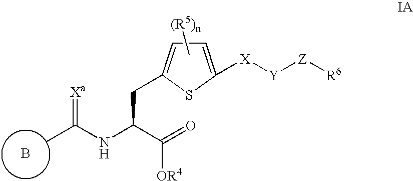
wherein:
n, B, R⁴, R⁵, R⁶, X^{a}, X, Y and Z are as defined in claim 1;
or a pharmaceutically acceptable salt thereof.

3. A compound according to claim 1 of the Formula IB′: 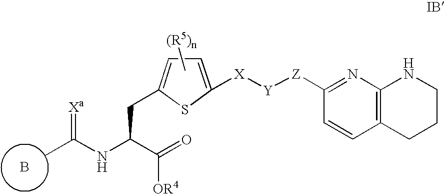
wherein:
n, B, R⁴, R⁵, X^{a}, X, Y and Z are as defined in claim 1;
or a pharmaceutically acceptable salt thereof.

4. A compound according to claim 1, or a pharmaceutically acceptable salt thereof, wherein —X—Y-Z- is —(CH₂)₃—.

5. A compound according to claim 1, or a pharmaceutically acceptable salt thereof, wherein —X—Y-Z- is *-O(CH₂)₂—, wherein * indicates the point of attachment to the thienyl group.

6. A compound according to claim 1, or a pharmaceutically acceptable salt thereof, wherein B is a 5 or 6 membered monocyclic heteroaryl ring or a fully aromatic bicyclic 9 or 10 membered heteroaryl ring, which heteroaryl ring contains from 1 to 4 atoms selected from O, S and N, and wherein B is attached to the C(X^{a}) group by a ring carbon atom;
and wherein the heteroaryl ring B is substituted in an ortho position to the group C(X^{a}) by a R¹ substituent selected from fluoro, chloro, bromo, (1-3C)alkyl, cyclopropyl and trifluoromethyl (for example R¹ is selected from chloro, bromo, (1-3C)alkyl, cyclopropyl and trifluoromethyl);
and wherein the heteroaryl ring B optionally bears on carbon 1 or more (for example 1, 2 or 3) R³ substituents selected from halo, trifluoromethyl (1-4C)alkyl, (2-4C)alkenyl, (2-4C)alkynyl and (1-4C)alkoxy,
or two adjacent substituents on ring B optionally form a (1-3C)alkylenedioxy group,
and wherein if the heteroaryl ring B contains an —NH— moiety, the nitrogen of said moiety optionally bears a substituent selected from (1-4C)alkyl, (2-4C)alkenyl and (2-4C)alkynyl;
and wherein any carbon containing substituent on the heteroaryl ring B optionally bears on carbon one or more substituents selected from hydroxy and (1-4C)alkoxy.

7. A compound according to claim 1, or a pharmaceutically acceptable salt thereof, wherein B is selected from 3-chloro-2-thienyl, 3-methyl-2-thienyl, 2-methyl-3-thienyl, 2-ethyl-3-thienyl, 2-methoxy-3-thienyl, 4-methyl-3-thienyl, 4-ethyl-3-thienyl, 4-methoxy-3-thienyl, 3-chloro-4-methyl-2-thienyl, 2,5-dichloro-3-thienyl, 1-methyl-1H-pyrrol-2-yl, 3-methyl-1H-pyrazol-4-yl, 3,5-dimethyl-1H-pyrazol-4-yl, 1,3,5-trimethyl-1H-pyrazol-4-yl, 1-ethyl-3-methyl-1H-pyrazol-5-yl, 3-methyl-2-furyl, 2-methyl-3-furyl, 4-methyl-1,3-thiazol-5-yl, 2,4-dimethyl-1,3-thiazol-5-yl, 3-methylisoxazol-4-yl, 3,5-dimethylisoxazol-4-yl, 2,5-dimethyl-1,3-oxazol-4-yl, 4-methyl-1,3-oxazol-5-yl, 3-chloropyridin-2-yl, 3-methylpyridin-2-yl, 3,6-dichloropyridin-2-yl, 3,5-dichloropyridin-2-yl, 3-chloro-5-(trifluoromethyl)pyridin-2-yl, 2-chloro-6-methylpyridin-3-yl, 4-methylpyridin-3-yl, 4-chloropyridin-3-yl, 2-methylpyridin-3-yl, 2-chloropyridin-3-yl, 3-chloropyridin-4-yl, 3-bromopyridin-4-yl, 3-methoxypyridin-4-yl, 3-methylpyridin-4-yl, 3-ethylpyridin-4-yl, 3-(methylthio)pyridin-4-yl, 3-(ethylthio)pyridin-4-yl, 3,5-dichloropyridin-4-yl, 3,5-dibromopyridin-4-yl, 3,5-dimethylpyridin-4-yl, 3,5-diethylpyridin-4-yl, 2-(ethylthio)pyridin-3-yl, 2,6-dichloropyridin-3-yl, 2,6-dimethoxypyridin-3-yl, 3-chloro1-methyl-1H-indol-2-yl and 3-methyl-1-benzofuran-2-yl. Particularly B is selected from 3-chloro-2-thienyl, 3-methyl-2-thienyl, 3-chloro-4-methyl-2-thienyl, 2,5-dichloro-3-thienyl, 1-methyl-1H-pyrrol-2-yl, 3-methyl-1H-pyrazol-4-yl, 3,5-dimethyl-1H-pyrazol-4-yl, 1,3,5-trimethyl-1H-pyrazol-4-yl, 1-ethyl-3-methyl-1H-pyrazol-5-yl, 3-methyl-2-furyl, 2-methyl-3-furyl, 4-methyl-1,3-thiazol-5-yl, 2,4-dimethyl-1,3-thiazol-5-yl, 3-methylisoxazol-4-yl, 3,5-dimethylisoxazol-4-yl, 2,5-dimethyl-1,3-oxazol-4-yl, 4-methyl-1,3-oxazol-5-yl, 3-chloropyridin-2-yl, 3-methylpyridin-2-yl, 3,6-dichloropyridin-2-yl, 3,5-dichloropyridin-2-yl, 3-chloro-5-(trifluoromethyl)pyridin-2-yl, 2-chloro-6-methylpyridin-3-yl, 4-methylpyridin-3-yl, 2-methylpyridin-3-yl, 2-chloropyridin-3-yl, 3-methylpyridin-4-yl, 3,5-dichloropyridin-4-yl, 2-(ethylthio)pyridin-3-yl, 2,6-dichloropyridin-3-yl, 2,6-dimethoxypyridin-3-yl and 3-methyl-1-benzofuran-2-yl.

8. A compound according to claim 7, or a pharmaceutically acceptable salt thereof, wherein —X—Y-Z- is —(CH₂)₃—.

9. A compound according to claim 7, or a pharmaceutically acceptable salt thereof, wherein —X—Y-Z- is *-O(CH₂)₂—, wherein * indicates the point of attachment to the thienyl group.

10. A compound according to claim 1, or a pharmaceutically acceptable salt thereof, wherein B is selected from 3-chloro-2-thienyl, 3,5-dimethylisoxazol-4-yl and 3,5-dichloropyridin-4-yl; and —X—Y-Z- is *-O(CH₂)₂—, wherein * indicates the point of attachment to the thienyl group.

11. A compound according to claim 1, or a pharmaceutically acceptable salt thereof, wherein R⁴ is hydrogen.

12. A compound according to claim 1, or a pharmaceutically acceptable salt thereof, wherein n=0.

13. A compound according to claim 1, or a pharmaceutically acceptable salt thereof, wherein X^{a} is oxygen.

14. A compound selected from:
N-[(3,5-dimethylisoxazol-4-yl)carbonyl]-3-(5-{3-[6-(methylamino)pyridin-2-yl]propyl}thiophen-2-yl)-L-alanine; and
N-[(3,5-dichloropyridin-4-yl)carbonyl]-3-(5-{3-[6-(methylamino)pyridin-2-yl]propyl}thiophen-2-yl)-L-alanine;
or a pharmaceutically acceptable salt thereof.

15. N-[(3,5-dimethylisoxazol-4-yl)carbonyl]-3-{5-[3-(5,6,7,8-tetrahydro-1,8-naphthyridin-2-yl)propyl]thiophen-2-yl}-L-alanine, or a pharmaceutically acceptable salt thereof.

16. N-(3,5-dichloroisonicotinoyl)-3-{5-[3-(5,6,7,8-tetrahydro-1,8-naphthyridin-2-yl)propyl]-thiophen-2-yl}-L-alanine, or a pharmaceutically acceptable salt thereof.

17. N-(3,5-dichloroisonicotinoyl)-3-{5-[2-(5,6,7,8-tetrahydro-1,8-naphthyridin-2-yl)ethoxy]-2-thienyl}-L-alanine, or a pharmaceutically acceptable salt thereof

18. A pharmaceutical composition which comprises a compound of the formula I, or a pharmaceutically acceptable thereof, as defined in claim 1 in association with a pharmaceutically-acceptable diluent or carrier.

19. A method of treatment of a disease mediated in part or alone by a5b1 comprising administering to an animal or human in need of said treatment a therapeutically effective amount of a compound of formula I, or a pharmaceutically acceptable salt thereof, as defined in claim 1.

20. A method of treatment of a human or animal suffering from cancer comprising administering to said human or animal a therapeutically effective amount of a compound of formula I, or a pharmaceutically acceptable salt thereof, as defined in claim 1.

21. The method according to claim 20 wherein the cancer is selected from carcinoma of the breast, ovary, lung (including small cell lung cancer, non-small cell lung cancer and bronchioalveolar cancer), colon, rectum, prostate, bile duct, bone, bladder, head and neck, kidney, liver, gastrointestinal tissue, oesophagus, pancreas, skin, testes, thyroid, uterus, cervix, vulva, leukemia, lymphoma, tumours of the central and peripheral nervous system, melanoma, multiple myeloma, fibrosarcoma, osteosarcoma and malignant brain tumours.

22. A method of inhibiting pathological angiogenesis in a human or animal comprising administering to said human or animal in need of said inhibiting a therapeutically effective amount of a compound of formula I or a pharmaceutically acceptable salt, as defined in claim 1.

23. A method of treatment of a human or animal suffering from cancer comprising administering to said human or animal a therapeutically effective amount of a compound of formula I, or a pharmaceutically acceptable salt thereof, as defined in claim 1, and one or more additional chemotherapeutic agent.

24. A process for the preparation of a compound of the formula I, or a pharmaceutically acceptable thereof, as defined in claim 1, which process comprises:
Process (a) for the preparation of those compounds of formula I wherein Z is N(R²⁶), O or S, by reacting a compound of the formula II: 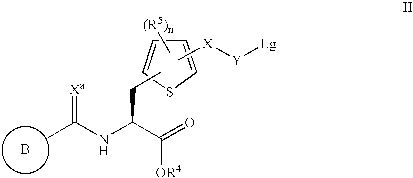
wherein B, R⁴, R⁵, X^{a}, A, Z, Y and n are as defined in claim 1, except any functional group is protected if necessary, and
Lg is a displaceable group,
with a compound of the formula III:
R⁶-ZH III
wherein R⁶ and Z are as defined in claim 1, except any functional group is protected if necessary; or
Process (b) for the preparation of those compounds of the formula I wherein X is O, the coupling of a compound of the formula IV: 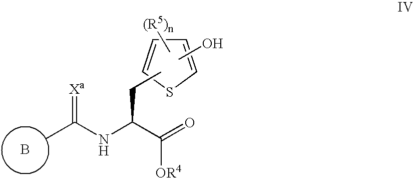
wherein B, R⁴, R⁵, X^{a}, and n are as defined in claim 1, except any functional group is protected if necessary,
with a compound of the formula V:
R⁶-Z-Y—OH V
wherein R⁶, Y and Z are as defined in claim 1, except any functional group is protected if necessary; or
Process (c) for the preparation of those compounds of formula I wherein X is O, N(R²⁶) or S by reacting a compound of the formula VI: 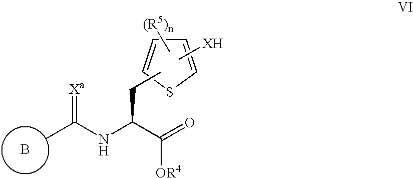
wherein B, R⁴, R⁵, X^{a} and n are as defined in claim 1, except any functional group is protected if necessary,
with a compound of the formula VII:
R⁶-Z-Y-Lg¹ VII
wherein R⁶, Y and Z are as defined in claim 1, except any functional group is protected if necessary, and
Lg¹ is a displaceable group; or
Process (d) for the preparation of those compounds of the formula I wherein Z is wherein Z is —C═C—, —C≡C— or the group —Y-Z is alkylene, the reaction of a compound of the formula VIII: 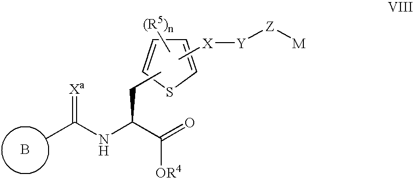
wherein B, R⁴, R⁵, X^{a}, X, Y and n are as defined in claim 1, except any functional group is protected if necessary,
and M is a suitable displaceable group,
with a compound of the formula R⁶ Lg²,
wherein R⁶ is as defined in claim 1, except any functional group is protected if necessary, and
Lg² is a displaceable group; or
Process (e) for the preparation of those compounds of the formula I wherein X is N(R²⁶)C(O), the coupling of a compound of the formula IX: 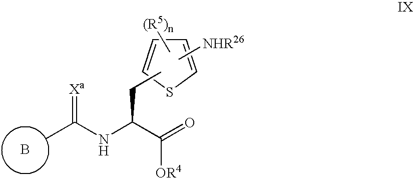
wherein B, R⁴, R⁵, R²⁶, X^{a} and n are as defined in claim 1, except any functional group is protected if necessary,
with a compound of the formula X, or a reactive derivative thereof:
R⁶-Z-Y—C(O)OH X
wherein R⁶, Y and Z are as defined in claim 1, except any functional group is protected if necessary; or
Process (f) for compounds of formula (I) where X^{a} is oxygen, the coupling of a compound of the formula XI: 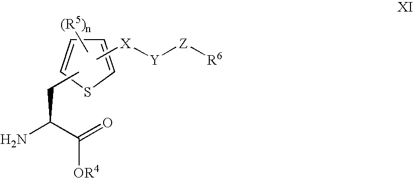
wherein R⁴, R⁵, R⁶, X, Y, Z and n are as defined in claim 1, except any functional group is protected if necessary,
with a compound of the formula XII, or a reactive derivative thereof: 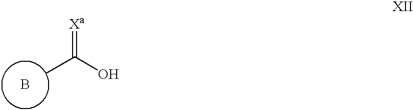
wherein B is as hereinbefore defined in claim 1, except any functional group is protected if necessary; or
Process (g) for the preparation of those compounds of the formula I wherein X is C(O)N(R²⁶), the coupling of a compound of the formula XIII, or a reactive derivative thereof: 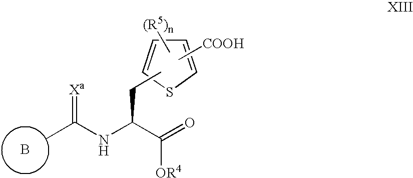
wherein B, R⁴, R⁵, X^{a}, and n are as defined in claim 1, except any functional group is protected if necessary,
with a compound of the formula XIV:
R⁶-Z-Y—NH(R²⁶) XIV
wherein R⁶, Y, Z and R²⁶ are as defined in claim 1, except any functional group is protected if necessary; or
Process (h) for the preparation of those compounds of the formula I wherein X is N(R²⁶), O or S, the coupling of a compound of the formula XV: 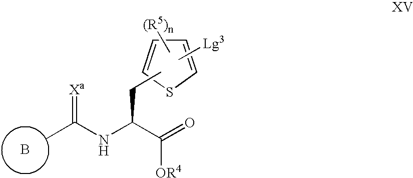
wherein B, R⁴, R⁵, X^{a} and n are as defined in claim 1, except any functional group is protected if necessary, and
Lg³ is a suitable displaceable group,
with a compound of the formula XVI:
R⁶-Z-Y—X—H XVI
wherein R⁶, X, Y and Z are as defined in claim 1, except any functional group is protected if necessary; or
Process (i) for the preparation of those compounds of the formula I wherein X is —C═C—, —C≡C— or the group —X—Y is alkylene, the coupling of a compound of the formula XV: 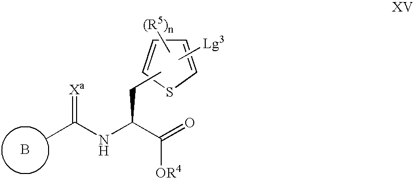
wherein B, R⁴, R⁵, X^{a}, Lg³ and n are as defined in claim 1, except any functional group is protected if necessary,
with a compound of the formula XVII:
R⁶-Z-Y—X-M XVII
wherein R⁶, Y and Z are as defined in claim 1, except any functional group is protected if necessary,
and M is a suitable displaceable group; or
Process (j) for the preparation of those compounds of the formula I wherein Z is N(R²⁶), the coupling of a compound of the formula XVIII: 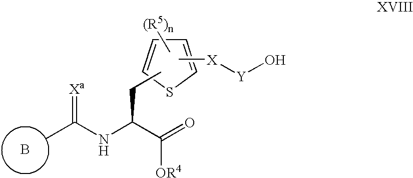
wherein B, R⁴, R⁵, A, X^{a}, X, Y and n are as defined in claim 1, except any functional group is protected if necessary,
with a compound of the formula XIX:
R⁶—N(R²⁶)H XIX
wherein R⁶ and R²⁶ are as defined in claim 1, except any functional group is protected if necessary; or
Process (k) for the preparation of those compounds of formula I wherein Z is N(R²⁶), O or S, by reacting a compound of the formula XX: 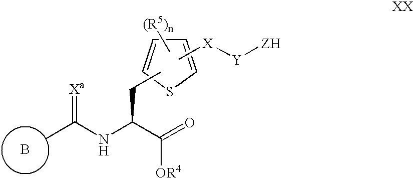
wherein B R⁴, R⁵, X^{a}, X, Y, Z and n are as defined in claim 1, except any functional group is protected if necessary,
with a compound of the formula XXI:
R⁶-Lg XXI
wherein R⁶ is as defined in claim 1, except any functional group is protected if necessary, and
Lg is a displaceable group; or
Process (1) for the preparation of those compounds of the formula I wherein the group —X—Y-Z- contains an alkylene chain of at least 3 carbon atoms in length, the hydrogenation of the product of Process (d) or (i) described herein; or
Process (m) for the preparation of those compounds of the formula I where Xa is a sulfur, by reacting a compound of the formula (I) of the formula XXII: 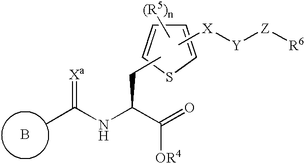
wherein B, R⁴, R⁵, R⁶, X, Y, Z and n are as defined in claim 1, except any functional group is protected if necessary,
with a thiation reagent;
Process (n) for the preparation of those compounds of the formula I wherein —X—Y-Z- contains a (1-6C)alkoxy or substituted (1-6C)alkoxy group or a (1-6C)alkylamino or substituted (1-6C)alkylamino group, the alkylation, conveniently in the presence of a suitable base, of the corresponding alcohol or amine in which X, Y or Z contains a hydroxy group or a primary or secondary amino group as appropriate; or a reductive amination in which X, Y or Z contains a primary or secondary amino group as appropriate; or
Process (o) when R⁶ is 5,6,7,8-tetrahydro-1,8-naphthyridin-2-yl; the reduction of a compound of the formula XXIV: 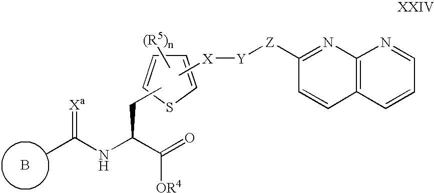
wherein B, R¹, R², R³, R⁴, R⁵, X, Y, Z, n and m are as defined in claim 1, except any functional group is protected if necessary;
and thereafter, optionally (in any order):
(i) converting a compound of the formula I into another compound of the formula I;
(ii) removing any protecting groups; and
(iii) forming a pharmaceutically acceptable salt of the compound of formula I.
